SEQUENCE LISTING (154 sequences)

SEQ ID NO: 1 - protein, 20 aa, synthetic construct
METDTLLLWVLLLWVPGSTG

SEQ ID NO: 2 - protein, 17 aa, synthetic construct
MRAWIFFLLCLAGRALA

SEQ ID NO: 3 - protein, 25 aa, synthetic construct
MTMYLWLKLLAFGFAFLDTEVFVTG

SEQ ID NO: 4 - protein, 238 aa, synthetic construct
METDTLLLWVLLLWVPGSTGDIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 471 aa, synthetic construct
METDTLLLWVLLLWVPGSTGEVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 465 aa, synthetic construct
MRAWIFFLLCLAGRALAEVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 235 aa, synthetic construct
MRAWIFFLLCLAGRALADIVMTQSPDSLAVSLGERATINCRASKSVSTSGYSYLHWYQQKPGQPPKLLIYLASNLESGVPDRFSGSGSGTDFTLTISSLQAEDVAVYYCQHSRELPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 468 aa, synthetic construct
MRAWIFFLLCLAGRALAEVQLVESGGGLVQPGGSLRLSCAASGFDFSRYWMSWVRQAPGKGLVWVSEINPTSSTINFTPSLKDRFTISRDNAKNTLYLQMNSLRAEDTAVYYCARGNYYRYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 465 aa, synthetic construct
MRAWIFFLLCLAGRALAEVQLVESGGGLVQPGGSLRLSCAASGFDFSRYWMSWVRQAPGKGLVWVSEINPTSSTINFTPSLKDRFTISRDNAKNTLYLQMNSLRAEDTAVYYCARGNYYRYGDAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 235 aa, synthetic construct
MRAWIFFLLCLAGRALADIAMTQSPDSLAVSLGERATINCRASKSVSTSGYSYLHWYQQKPGQPPKLLIYLASNLESGVPDRFSGSGSGTDFTLTISSLQAEDVATYYCQHSRELPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 468 aa, synthetic construct
MRAWIFFLLCLAGRALAEVQLVESGGGLVQPGGSLRLSCAASGFDFSRYWMSWVRQAPGKGLEWIGEINPTSSTINFTPSLKDKVFISRDNAKNTLYLQMNSLRAEDTAVYYCARGNYYRYGDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 465 aa, synthetic construct
MRAWIFFLLCLAGRALAEVQLVESGGGLVQPGGSLRLSCAASGFDFSRYWMSWVRQAPGKGLEWIGEINPTSSTINFTPSLKDKVFISRDNAKNTLYLQMNSLRAEDTAVYYCARGNYYRYGDAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 641 aa, synthetic construct
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSGGGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 14 - DNA, 717 bp, synthetic construct
  source 1..717: mol_type = other DNA
atggagacagatactttgctcctttgggtactcttgctgtgggttcccggtagcaccggcgacatcgctctcactcagagcccagcttcactcgcggtcagcttgggacaaagagcgacaatatcctgcagagctagtaagtctgtgagcacgagcggttactcatacttgcactggtatcaacaaaagcccggtcaaccgcctaaattgttgatatatttggcgtcaaatctcgagagtggggttccagctcgcttcagtggctctggaagtgggaccgacttcaccctcaacattcacccggtagaagaggaagatgcagctacatattactgccagcacagccgggagttgccttttacctttggctctggtacgaaattggagataaagcgaacggtagcggcgccatccgtttttatatttccgccaagtgatgagcaacttaaaagtgggactgcgagcgtcgtatgcctcttgaacaacttctatcctcgggaggctaaggtccaatggaaggttgataatgcgctccagtccgggaactctcaggaatcagtgaccgaacaagattccaaagactcaacttacagcctgtcttccacgctcactctcagcaaagccgattatgaaaagcataaggtgtatgcttgtgaagttacacaccaaggtctctccagccccgtaaccaagtcattcaacagaggggaatgttga

SEQ ID NO: 15 - DNA, 1416 bp, synthetic construct
  source 1..1416: mol_type = other DNA
atggagacagatactttgctcctttgggtactcttgctgtgggttcccggtagcaccggcgaagtgaaactgctcgagtcaggtggggggctcgtccagccgggtggttcactgaagctctcttgtgctgcatctggattcgatttcagcaggtactggatgtcttgggttcggcaggctccaggaaaaggcctggaatggatcggggaaataaaccccacgtcaagcactatcaatttcaccccctcattgaaggataaagtgttcatctccagggataatgcgaagaatacactctatctgcagatgagcaaggtaaggagtgaggacacagcactttactactgtgcaaggggtaactactaccgatatggtgatgcaatggactattggggacaagggacgagcgtgactgtctcttctgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 16 - DNA, 1398 bp, synthetic construct
  source 1..1398: mol_type = other DNA
atgagagcctggatcttcttcctgttgtgcctggctggtagggcactcgcagaagtgaaactgctcgagtcaggtggggggctcgtccagccgggtggttcactgaagctctcttgtgctgcatctggattcgatttcagcaggtactggatgtcttgggttcggcaggctccaggaaaaggcctggaatggatcggggaaataaaccccacgtcaagcactatcaatttcaccccctcattgaaggataaagtgttcatctccagggataatgcgaagaatacactctatctgcagatgagcaaggtaaggagtgaggacacagcactttactactgtgcaaggggtaactactaccgatatggtgatgcaatggactattggggacaagggacgagcgtgactgtctcttctgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 17 - DNA, 708 bp, synthetic construct
  source 1..708: mol_type = other DNA
atgagagcctggatcttcttcctgttgtgcctggctggtagggcactcgcagacattgtaatgacacagagcccggactcattggcggtgagtttgggtgaaagagcaacgatcaactgtcgggcatcaaaatctgtttccaccagtggctatagctatcttcattggtatcaacaaaagccgggccagccgccaaaattgcttatatatttggctagcaatttggaatcaggagttcctgaccggttttcagggtcaggtagtggcacggattttacactgacgatctctagtcttcaagccgaggacgtcgcagtgtactattgccagcactctcgggaactgccattcacattcggtggtgggacgaaagtagaaatcaaacgaacagtggcggcaccgtccgtatttattttccctccaagtgatgagcagctcaaaagtggcacagcgagcgttgtatgtctcctcaataacttttaccccagagaggcgaaagtacaatggaaggttgataacgcactccaaagcggtaactctcaggagagtgtcacagaacaggatagtaaagactccacctactccctcagttctacccttactctttcaaaagcggattatgagaaacacaaggtttacgcctgtgaagtcactcatcagggactgagttccccggtcacgaagtcatttaatcgcggtgagtgctga

SEQ ID NO: 18 - DNA, 1407 bp, synthetic construct
  source 1..1407: mol_type = other DNA
atgagagcctggatcttcttcctgttgtgcctggctggtagggcactcgcagaggtgcaactggttgaatcaggaggtgggcttgtgcagcctgggggaagtttgaggctgtcttgcgctgcatccgggtttgacttttctaggtattggatgagttgggtgaggcaagcaccaggcaaagggctcgtgtgggtgtctgagattaatccaacgagtagtacgatcaattttaccccctcactcaaggaccgctttacaatcagccgggacaatgcaaagaatactctgtacctccaaatgaacagtttgcgagcagaggatacggccgtctattattgcgcaaggggaaactactaccgctacggagacgcaatggactattggggtcaggggaccaccgtgaccgtttcttccgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 19 - DNA, 1398 bp, synthetic construct
  source 1..1398: mol_type = other DNA
atgagagcctggatcttcttcctgttgtgcctggctggtagggcactcgcagaggtgcaactggttgaatcaggaggtgggcttgtgcagcctgggggaagtttgaggctgtcttgcgctgcatccgggtttgacttttctaggtattggatgagttgggtgaggcaagcaccaggcaaagggctcgtgtgggtgtctgagattaatccaacgagtagtacgatcaattttaccccctcactcaaggaccgctttacaatcagccgggacaatgcaaagaatactctgtacctccaaatgaacagtttgcgagcagaggatacggccgtctattattgcgcaaggggaaactactaccgctacggagacgcaatggactattggggtcaggggaccaccgtgaccgtttcttccgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 20 - DNA, 708 bp, synthetic construct
  source 1..708: mol_type = other DNA
atgagagcctggatcttcttcctgttgtgcctggctggtagggcactcgcagatattgctatgacccagtcccctgatagcttggccgtgagcctgggagagcgagcgactatcaattgccgcgcgagcaagagcgtctctacttccggctactcttacctccattggtatcaacagaagccagggcagccaccgaagctccttatctaccttgcgtcaaacttggagagcggcgtgcccgaccggttcagcgggtcaggcagcgggactgactttacgcttactatctcctctctgcaagccgaagacgtggctacttattactgtcagcacagtcgggagctgcccttcacatttggaggaggaaccaaagtggaaatcaagaggacagtagcagcgccaagtgtcttcatctttcccccgagcgacgagcagctcaagtccggtactgccagtgttgtatgccttttgaataacttctatccacgcgaggcaaaagtacagtggaaagtggataatgcactccagagtggtaactcacaagaatccgtaaccgagcaagattccaaggattccacctactcactgtccagtacgcttacactcagtaaggctgactacgaaaagcataaagtttacgcttgtgaagtcacgcaccaaggtcttagttccccagtaacgaagtcatttaatcgcggtgagtgctga

SEQ ID NO: 21 - DNA, 1407 bp, synthetic construct
  source 1..1407: mol_type = other DNA
atgagagcctggatcttcttcctgttgtgcctggctggtagggcactcgcagaagtccagttggttgagagtggcggaggccttgttcaacctggaggcagtctcagactctcatgtgcggcttctggattcgacttctctcgatactggatgtcctgggtacgacaggcacccggaaagggcttggaatggatcggggaaattaaccccacatccagcacaataaacttcacgccgagccttaaggataaggttttcatttcccgagacaatgctaagaacactctttatctccagatgaactcacttcgagccgaagacactgcggtatattattgcgctaggggtaattactaccgatacggggatgcgatggattattggggtcaggggaccaccgtgaccgtttcttccgctagcaccaaaggaccctccgtctttccgctcgcgccgagcagcaagtccacgagcggggggacagccgcccttgggtgtctggtcaaggattattttcctgagcccgttactgtctcctggaatagcggtgccttgacttctggggtacataccttcccggcggttttgcaatcatcaggactgtatagcctctctagcgttgtaacggtaccatcttcatcacttggcacacaaacatatatttgtaatgtaaatcacaaaccttcaaatactaaagttgacaaaaaagtggagccgaagagctgtgataaaacgcatacttgcccgccttgccccgctccagaattgctgggcgggccgtctgtgttcctcttcccaccgaaaccgaaggatacgctgatgataagtcgaacgcccgaagtaacatgcgtggttgttgatgtaagccacgaagatccggaagtaaagttcaactggtacgtagatggagtagaagtgcataacgcgaaaactaagccccgcgaggagcaatataattcaacctatcgggttgttagtgtgctcaccgttctccatcaggattggcttaacgggaaagagtataagtgcaaggttagcaataaagcattgccagcacctatagagaagacaattagcaaagcgaagggacagccacgagagccccaagtctacacgctgcctcccagccgcgacgagttgacgaagaatcaagtgtcactcacatgtcttgtcaagggattctatccctcagatattgcagtagaatgggagtctaatggacaaccagagaacaactataaaactactccaccggtgcttgatagtgacggtagttttttcctttatagtaagctgactgtcgataagagtcgctggcaacagggaaatgttttttcttgtagtgttatgcatgaagcacttcataaccattacacacaaaagtctctgtcactttcccctggcaaatga

SEQ ID NO: 22 - DNA, 1398 bp, synthetic construct
  source 1..1398: mol_type = other DNA
atgagagcctggatcttcttcctgttgtgcctggctggtagggcactcgcagaagtccagttggttgagagtggcggaggccttgttcaacctggaggcagtctcagactctcatgtgcggcttctggattcgacttctctcgatactggatgtcctgggtacgacaggcacccggaaagggcttggaatggatcggggaaattaaccccacatccagcacaataaacttcacgccgagccttaaggataaggttttcatttcccgagacaatgctaagaacactctttatctccagatgaactcacttcgagccgaagacactgcggtatattattgcgctaggggtaattactaccgatacggggatgcgatggattattggggtcaggggaccaccgtgaccgtttcttccgctagcaccaaaggaccctccgtctttccgctcgcgccgtgctctcgatccacgagcgagtctacggcggccttgggctgtctcgtgaaagactatttcccagagccagtgaccgtttcctggaactccggagcgctcacatcaggtgtgcatacctttccagccgtacttcaatcttcagggctgtatagcttgtcctctgtcgtgaccgtaccctccagttctctcgggacaaagacctatacctgtaacgtagaccataagccatccaataccaaagtagataagagggtggaatctaaatatggaccgccttgtccgccgtgcccggctcctgagttccttggagggccgtccgtctttctgtttccccctaaacctaaggacaccctgatgataagtaggacgccggaagtaacctgtgtcgtggttgatgtgtcacaggaagacccggaggtacaatttaattggtacgtcgatggagtcgaggtgcataatgcgaaaaccaagccacgagaggagcaattcaacagcacctaccgagtagtttcagtcttgactgtgctgcaccaagactggctgaatggcaaggagtacaaatgcaaggtcagtaataaaggattgccgtcttcaatagagaagactatcagtaaggcgaagggacaacctcgcgagcctcaggtatacactttgccgcccagccaagaagaaatgacgaagaaccaagtctctttgacttgcctggtcaaaggcttctacccctctgatatagcagtcgaatgggagtccaacggtcaaccggagaacaattataagacaactccacctgtgcttgattcagacggctcttttttcttgtatagccgccttaccgtagacaagagtcgatggcaggaaggtaatgtattttcttgtagtgtaatgcacgaggcacttcataatcactacacacaaaagtctctgtcactttccctgggcaaatga

SEQ ID NO: 23 - DNA, 1926 bp, synthetic construct
  source 1..1926: mol_type = other DNA
atgacaatgtacctttggctcaaattgctcgctttcgggttcgctttccttgatacagaggtcttcgttacgggtcaatcacccacgccctccccgacggacgcatacctgaatgcttcagaaactacaacgctgagcccaagtggttcagctgttatatcaactactactattgctaccacgcccagcaaacccacttgcgacgaaaagtatgcgaacataaccgtagactatttgtataacaaagagaccaaacttttcacagcaaaattgaatgttaacgaaaatgttgaatgtgggaataacacatgtacgaataacgaggtccataatttgacggagtgcaagaatgcgtcagtctctatctcacataactcctgcaccgcgcctgacaagacccttatccttgacgttcctcccggggtagaaaagttccaacttcacgactgtacccaagtggagaaggcagacacgacaatttgccttaagtggaagaacattgaaaccttcacgtgcgacactcaaaatatcacttaccggttccaatgcggcaacatgatctttgataataaggaaattaaacttgaaaatctggaacccgagcatgagtataaatgtgactctgaaattttgtataacaatcacaagtttaccaacgctagcaaaatcattaaaaccgattttggttcaccgggtgaacctcaaattatattctgccggtctgaagcggcccatcaaggcgttattacatggaatccacctcaacgctctttccataatttcacattgtgctatataaaagagacggaaaaagactgtcttaatctcgacaagaatctcatcaagtacgacttgcagaacctcaagccttacacaaaatatgtgctgtccttgcacgcttatataatagcgaaagtacaacgcaacgggtcagctgcgatgtgtcacttcaccactaagtccgcaccgccatctcaggtttggaatatgaccgtctcaatgacgagcgacaactcaatgcacgttaaatgtcgcccaccacgggatcgaaatggcccgcatgaaagataccacctcgaagtcgaagcggggaatacccttgtcaggaatgaatcacacaaaaactgtgatttccgagtaaaagacctgcaatatagcactgattacacgttcaaggcgtacttccataatggagactatccgggcgagcctttcatcttgcatcattccacaagttacaacagtggaggtggatccggttgtaagccctgtatttgcacagtaccagaggtgtcttctgtttttatatttcctccaaagcctaaggatgtactcactataactcttacaccaaaggttacttgtgtggttgtcgatatcagcaaggatgacccggaggtacaatttagttggtttgtagatgatgtggaagtccacaccgcgcaaacgcagccacgagaagaacagtttaactctacatttaggagcgttagcgagctcccaatcatgcaccaggattggcttaacggaaaggagtttaaatgccgggtcaactcagcggcttttccagccccgattgagaagacaatttcaaaaaccaagggacgcccaaaagccccgcaagtatatactatcccaccaccaaaggaacaaatggctaaggataaagttagtctgacttgtatgattacagattttttcccagaggatataacagtcgaatggcagtggaatggtcagccggcagaaaactataaaaacacccagccgatcatgaatacaaacggttcctactttgtttatagcaagttgaatgtccaaaaaagcaactgggaagcgggcaatacctttacctgttccgtgctgcacgagggactgcataaccatcacaccgaaaagagtctgtcacatagtcctggaaaatga

SEQ ID NO: 24 - protein, 222 aa, synthetic construct
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 25 - protein, 1306 aa, synthetic construct
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 26 - protein, 107 aa, synthetic construct [sequence omitted]

SEQ ID NO: 27 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 28 - protein, 330 aa, synthetic construct [sequence omitted]

SEQ ID NO: 29 - protein, 225 aa, synthetic construct
THTCPPCPAPEFFGGPSVFFFPPKPKDTFMISRTPEVTCVVVDVSHEDPEVKFNWYVDGVEVHNAKTKPREEQYNSTYRVVSVETVFHQDWENGKEYKCKVSNKAFPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 30 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 31 - protein, 217 aa, synthetic construct
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 33 - protein, 218 aa, synthetic construct
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 34 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 218 aa, synthetic construct
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 15 aa, synthetic construct
RASKSVSTSGYSYLH

SEQ ID NO: 38 - protein, 8 aa, synthetic construct
YLASNLES

SEQ ID NO: 39 - protein, 9 aa, synthetic construct
QHSRELPFT

SEQ ID NO: 40 - protein, 13 aa, synthetic construct
AASGFDFSRYWMS

SEQ ID NO: 41 - protein, 10 aa, synthetic construct
EINPTSSTIN

SEQ ID NO: 42 - protein, 14 aa, synthetic construct
ARGNYYRYGDAMDY

SEQ ID NO: 43 - protein, 10 aa, synthetic construct
KSVSTSGYSY

SEQ ID NO: 44 - protein, 8 aa, synthetic construct
GFDFSRYW

SEQ ID NO: 45 - protein, 8 aa, synthetic construct
INPTSSTI

SEQ ID NO: 46 - protein, 7 aa, synthetic construct
LASNLES

SEQ ID NO: 47 - protein, 10 aa, synthetic construct
GFDFSRYWMS

SEQ ID NO: 48 - protein, 17 aa, synthetic construct
EINPTSSTINFTPSLKD

SEQ ID NO: 49 - protein, 12 aa, synthetic construct
GNYYRYGDAMDY

SEQ ID NO: 50 - protein, 7 aa, synthetic construct
GFDFSRY

SEQ ID NO: 51 - protein, 6 aa, synthetic construct
NPTSST

SEQ ID NO: 52 - protein, 7 aa, synthetic construct
YSYLHWY

SEQ ID NO: 53 - protein, 10 aa, synthetic construct
LLIYLASNLE

SEQ ID NO: 54 - protein, 8 aa, synthetic construct
QHSRELPF

SEQ ID NO: 55 - protein, 6 aa, synthetic construct
SRYWMS

SEQ ID NO: 56 - protein, 13 aa, synthetic construct
WIGEINPTSSTIN

SEQ ID NO: 57 - protein, 13 aa, synthetic construct
ARGNYYRYGDAMD

SEQ ID NO: 58 - protein, 13 aa, synthetic construct
WVSEINPTSSTIN

SEQ ID NO: 59 - protein, 111 aa, synthetic construct
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 60 - protein, 121 aa, synthetic construct
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSS

SEQ ID NO: 61 - protein, 111 aa, synthetic construct
DIVMTQSPDSLAVSLGERATINCRASKSVSTSGYSYLHWYQQKPGQPPKLLIYLASNLESGVPDRFSGSGSGTDFTLTISSLQAEDVAVYYCQHSRELPFTFGGGTKVEIK

SEQ ID NO: 62 - protein, 121 aa, synthetic construct
EVQLVESGGGLVQPGGSLRLSCAASGFDFSRYWMSWVRQAPGKGLVWVSEINPTSSTINFTPSLKDRFTISRDNAKNTLYLQMNSLRAEDTAVYYCARGNYYRYGDAMDYWGQGTTVTVSS

SEQ ID NO: 63 - protein, 111 aa, synthetic construct
DIAMTQSPDSLAVSLGERATINCRASKSVSTSGYSYLHWYQQKPGQPPKLLIYLASNLESGVPDRFSGSGSGTDFTLTISSLQAEDVATYYCQHSRELPFTFGGGTKVEIK

SEQ ID NO: 64 - protein, 121 aa, synthetic construct
EVQLVESGGGLVQPGGSLRLSCAASGFDFSRYWMSWVRQAPGKGLEWIGEINPTSSTINFTPSLKDKVFISRDNAKNTLYLQMNSLRAEDTAVYYCARGNYYRYGDAMDYWGQGTTVTVSS

SEQ ID NO: 65 - protein, 29 aa, synthetic construct
GGGGSNWHEREINNISANINTEGEROFOR

SEQ ID NO: 66 - protein, 34 aa, synthetic construct
GGGSNGGGGSNWHEREINNISANINTEGEROFOR

SEQ ID NO: 67 - protein, 9 aa, synthetic construct
GGGSNGGSN

SEQ ID NO: 68 - protein, 33 aa, synthetic construct
GGGSNGGGGSWHEREINNISANINTEGEROFOR

SEQ ID NO: 69 - protein, 20 aa, synthetic construct
GGGGSGGGGSGGGGSGGGGS

SEQ ID NO: 70 - protein, 15 aa, synthetic construct
GGGGSGGGGSGGGGS

SEQ ID NO: 71 - protein, 10 aa, synthetic construct
GGGGSGGGGS

SEQ ID NO: 72 - protein, 5 aa, synthetic construct
GGGGS

SEQ ID NO: 73 - protein, 8 aa, synthetic construct
GGGSGGGS

SEQ ID NO: 74 - protein, 4 aa, synthetic construct
GGGS

SEQ ID NO: 75 - protein, 6 aa, synthetic construct
GGSGGS

SEQ ID NO: 76 - protein, 11 aa, synthetic construct
GGSGGGSGGSG

SEQ ID NO: 77 - protein, 10 aa, synthetic construct
GGSGGGSGSG

SEQ ID NO: 78 - protein, 8 aa, synthetic construct
GGSGGGSG

SEQ ID NO: 79 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 80 - protein, 192 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGDTTGSSVTLGCLVKGYFPEPVTLTWNSGSLSSGVHTFPAVLQSDLYTLSSSVTVTSS

SEQ ID NO: 81 - protein, 10 aa, synthetic construct
SASSSVSYMN

SEQ ID NO: 82 - protein, 7 aa, synthetic construct
DTSKLAS

SEQ ID NO: 83 - protein, 10 aa, synthetic construct
QQWSSNPFTF

SEQ ID NO: 84 - protein, 5 aa, synthetic construct
RYTMH

SEQ ID NO: 85 - protein, 17 aa, synthetic construct
YINPSRGYTNYNQKFKD

SEQ ID NO: 86 - protein, 8 aa, synthetic construct
YYDDHYCL

SEQ ID NO: 87 - protein, 121 aa, synthetic construct
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPXTFGGGTKLEIKRWHEREINXCAORN

SEQ ID NO: 88 - protein, 120 aa, synthetic construct
VQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 89 - protein, 11 aa, synthetic construct
HASQNIYVWLN

SEQ ID NO: 90 - protein, 7 aa, synthetic construct
KASNLHT

SEQ ID NO: 91 - protein, 9 aa, synthetic construct
QQGQTYPYT

SEQ ID NO: 92 - protein, 5 aa, synthetic construct
SYYIH

SEQ ID NO: 93 - protein, 17 aa, synthetic construct
CIYPGNVNTNYNEKFKD

SEQ ID NO: 94 - protein, 11 aa, synthetic construct
SHYGLDWNFDV

SEQ ID NO: 95 - protein, 7 aa, synthetic construct
RASQSVS

SEQ ID NO: 96 - protein, 6 aa, synthetic construct
ASNRAT

SEQ ID NO: 97 - protein, 10 aa, synthetic construct
QRSNWPPALT

SEQ ID NO: 98 - protein, 4 aa, synthetic construct
YYWS

SEQ ID NO: 99 - protein, 12 aa, synthetic construct
YGPGNYDWYFDL

SEQ ID NO: 100 - protein, 52 aa, synthetic construct
DITGAALLEAKEAAINELKQYGISDYYVTLINKAKTVEGVNALKAEILSALP

SEQ ID NO: 101 - protein, 45 aa, synthetic construct
LKEAKEKAIEELKKAGITSDYYFDLINKAKTVEGVNALKDEILKA

SEQ ID NO: 102 - protein, 39 aa, synthetic construct
RIARLEEKVKTLKAQNSELASTANMLREQVAQLKQKVMN

SEQ ID NO: 103 - protein, 39 aa, synthetic construct
LTDTLQAETDQLEDKKSALQTEIANLLKEKEKLEFILAA

SEQ ID NO: 104 - protein, 59 aa, synthetic construct
WREFCESHARAAALDFARRFRLYLASHPQYAGPGAEAAFSRRFAELFLQHFEAEVARAS

SEQ ID NO: 105 - protein, 55 aa, synthetic construct
THRAIFRFVPRHEDELELEVDDPLLVELQAEDYWYEAYNMRTGARGVFPAYYAIE

SEQ ID NO: 106 - protein, 101 aa, synthetic construct
LGEVHRFHVTVAPGTKLESGPATLHLCNDVLVLARDIPPAVTGQWKLSDLRRYGAVPSGFIFEGGTRCGYWAGVFFLSSAEGEQISFLFDCIVRGISPTKG

SEQ ID NO: 107 - protein, 115 aa, synthetic construct
DCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSS

SEQ ID NO: 108 - protein, 427 aa, synthetic construct
CAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGSQLFSYAAFSDSNIITVVVDTALYIAKQNSPVVEVWDKKTEKLCGLIDCVHFLREVMVKETKIFSFSNDFTIQKLIETRTNKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 109 - protein, 75 aa, synthetic construct
DQELKHLILEAADGFLFIVSCETGRVVYVSDSVTPVLNQQQSEWFGSTLYDQVHPDDVDKLREQLSTSENALTGR

SEQ ID NO: 110 - protein, 33 aa, synthetic construct
LSAKETKMLMAAGDKDGDGKIGVDEFSTLVAES

SEQ ID NO: 111 - protein, 44 aa, synthetic construct
SHIQIPPGLTELLQGYTVEVLRQQPPDLVEFAVEYFTRLREARA

SEQ ID NO: 112 - protein, 45 aa, synthetic construct
CGHIQIPPGLTELLQGYTVEVLRQQPPDLVEFAVEYFTRLREARA

SEQ ID NO: 113 - protein, 17 aa, synthetic construct
QIEYLAKQIVDNAIQQA

SEQ ID NO: 114 - protein, 21 aa, synthetic construct
CGQIEYLAKQIVDNAIQQAGC

SEQ ID NO: 115 - protein, 106 aa, synthetic construct
MGVQVETISPGDGRTFPKRGQTCWHYTGMLEDGKKFDSSRDRNPFKFMLGKQEVIRGWEEGVAQMSVGQRAKLTISPDYAYGATGHPGIIPPHATLVFDVELLKLE

SEQ ID NO: 116 - protein, 100 aa, synthetic construct
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 117 - protein, 353 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 118 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 119 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 120 - protein, 764 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 121 - protein, 585 aa, synthetic construct
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 122 - protein, 349 aa, synthetic construct
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 123 - protein, 18 aa, synthetic construct
VEPKSSDKTHTCPPCPAP

SEQ ID NO: 124 - protein, 255 aa, synthetic construct
VEPKSSDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 125 - protein, 453 aa, synthetic construct [sequence omitted]

SEQ ID NO: 126 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 127 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 128 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVAEEEWNTGETYTCVVAHEALPNRVTERTVDKSTGKPTLYNVSLVMSDTAGTCY

SEQ ID NO: 129 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSAEEWNTGETYTCVVAHEALPNRVTERTVDKSTGKPTLYNVSLVMSDTAGTCY

SEQ ID NO: 130 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEAEWNTGETYTCVVAHEALPNRVTERTVDKSTGKPTLYNVSLVMSDTAGTCY

SEQ ID NO: 131 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLAESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 132 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRASATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 133 - protein, 159 aa, synthetic construct
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 134 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 135 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCSTYDRNKCYTAVVPLVYGGETKMVETALTPDACYPD

SEQ ID NO: 136 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCRTYDRNKCYTAVVPLVYGGETKMVETALTPDACYPD

SEQ ID NO: 137 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYAYDRNKCYTAVVPLVYGGETKMVETALTPDACYPD

SEQ ID NO: 138 - protein, 137 aa, synthetic construct
QEDERIVLVDNKCKCARITSRIIRSSEDPNEDIVERNIRIIVPLNNREAISDPTSPLRTRFVYHLSDLCKKCDPTEVELDNQIVTATQSNICDEDSATETCYTYDRNKCYTAVVPLVYGGETKMVETALTPDACYPD

SEQ ID NO: 139 - protein, 136 aa, synthetic construct
EDERIVLVDNKCKCARITSRIIRSSEDPNEDIVERNIRIIVPLNNRENIADPTSPLRTRFVYHLSDLCKKCDPTEVELDNQIVTATQSNICDEDSATETCYTYDRNKCYTAVVPLVYGGETKMVETALTPDACYPD

SEQ ID NO: 140 - protein, 6 aa, synthetic construct
HHHHHH

SEQ ID NO: 141 - protein, 7 aa, synthetic construct
DYKDDDD

SEQ ID NO: 142 - protein, 8 aa, synthetic construct
DLYDDDDK

SEQ ID NO: 143 - protein, 15 aa, synthetic construct
GLNDIFEAQKIEWHE

SEQ ID NO: 144 - protein, 26 aa, synthetic construct
KRRWKKNFIAVSAANRFKKISSSGAL

SEQ ID NO: 145 - protein, 6 aa, synthetic construct
EEEEEE

SEQ ID NO: 146 - protein, 9 aa, synthetic construct
YPYDVPDYA

SEQ ID NO: 147 - protein, 10 aa, synthetic construct
EQKLISEEDL

SEQ ID NO: 148 - protein, 8 aa, synthetic construct
WRHPQFGG

SEQ ID NO: 149 - protein, 8 aa, synthetic construct
WSHPQFEK

SEQ ID NO: 150 - protein, 13 aa, synthetic construct
SLAELLNAGLGGS

SEQ ID NO: 151 - protein, 8 aa, synthetic construct
TQDPSRVG

SEQ ID NO: 152 - protein, 14 aa, synthetic construct
GKPIPNPLLGLDST

SEQ ID NO: 153 - protein, 797 aa, synthetic construct
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSGSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 154 - DNA, 2394 bp, synthetic construct
  source 1..2394: mol_type = other DNA
atgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatctggatccggttgtaagccctgtatttgcacagtaccagaggtgtcttctgtttttatatttcctccaaagcctaaggatgtactcactataactcttacaccaaaggttacttgtgtggttgtcgatatcagcaaggatgacccggaggtacaatttagttggtttgtagatgatgtggaagtccacaccgcgcaaacgcagccacgagaagaacagtttaactctacatttaggagcgttagcgagctcccaatcatgcaccaggattggcttaacggaaaggagtttaaatgccgggtcaactcagcggcttttccagccccgattgagaagacaatttcaaaaaccaagggacgcccaaaagccccgcaagtatatactatcccaccaccaaaggaacaaatggctaaggataaagttagtctgacttgtatgattacagattttttcccagaggatataacagtcgaatggcagtggaatggtcagccggcagaaaactataaaaacacccagccgatcatgaatacaaacggttcctactttgtttatagcaagttgaatgtccaaaaaagcaactgggaagcgggcaatacctttacctgttccgtgctgcacgagggactgcataaccatcacaccgaaaagagtctgtcacatagtcctggaaaatga

(19) United States
(12) Patent Application Publication
(10) Pub. No.: US 2025/0326856 A1
(43) Pub. Date: Oct. 23, 2025

(54) HUMANIZED ANTI-CD45 ANTIBODIES AND USES THEREOF

(71) Applicant: Fred Hutchison Cancer Center, Seattle, WA (US)

(72) Inventors: Roland B. Walter, Seattle, WA (US); Brenda M. Sandmaier, Mercer Island, WA (US); David J. King, Encinitas, CA (US); George S. Laszlo, Seattle, WA (US)

(73) Assignee: Fred Hutchison Cancer Center, Seattle, WA (US)

(21) Appl. No.: 18/870,336

(22) PCT Filed: May 31, 2023
(86) PCT No.: PCT/US2023/067721

§ 371 Date: Nov. 27, 2024

Related U.S. Application Data
(60) Provisional application No. 63/347,479, filed on May 31, 2022.

(51) Int. Cl. C07K 16/28; A61K 39/00; A61K 51/10; A61P 35/02

(52) U.S. Cl. CPC C07K 16/289; A61K 51/1096; A61P 35/02; A61K 2039/505; A61K 2121/00; C07K 2317/24; C07K 2317/565; C07K 2317/92

(57) ABSTRACT

Novel chimeric and/or humanized forms of the anti-CD45 BC8 antibody are described. The disclosed chimeric or humanized antibodies can be used as research, diagnostic, or therapeutic tools against CD45-related disorders, such as hematologic malignancies including acute myeloid leukemia (AML) and acute lymphoblastic leukemia (ALL), other myeloid and lymphoid disorders, other cancers, as well as non-malignant disorders, such as autoimmune disorders, infections, inherited blood disorders, and metabolic disorders.

### CROSS REFERENCE TO RELATED APPLICATION

This application is a U.S. National Phase Application based on International Patent Application No. PCT/US2023/067721, filed May 31, 2023, which claims priority to U.S. Provisional Patent Application No. 63/347,479, the entire contents of both of which are incorporated by reference herein.

### STATEMENT REGARDING FEDERALLY SPONSORED RESEARCH OR DEVELOPMENT

This invention was made with government support under CA078902 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3CA4766.XML. The file is 172,032 bytes, was created on Nov. 25, 2024 and is being submitted electronically via Patent Center.

### FIELD OF THE DISCLOSURE

The current disclosure provides novel chimeric or humanized forms of the anti-CD45 BC8 antibody. The disclosed antibodies have a wide variety of research, diagnostic, and therapeutic uses when working with cells of hematopoietic origin. Examples include in the treatment of hematologic malignancies, as lymphodepleting agents prior to adoptive cell therapy, in autologous transplant to restore hematopoietic capability, and/or to facilitate engraftment of normal or gene edited hematopoietic stem/progenitor cells in patients with non-malignant disorders undergoing hematopoietic cell transplantation (HCT), among other uses described elsewhere herein.

### BACKGROUND OF THE DISCLOSURE

CD45 is a transmembrane protein that is expressed by cells of hematopoietic origin, with the exception of mature erythrocytes and platelets, It is a signaling molecule that plays a key role in T-cell and B-cell receptor signal transduction. CD45 also regulates a number of cellular processes including cell growth and differentiation.

Due to its broad expression across cells of hematopoietic origin, anti-CD45 antibodies have numerous uses in treating pathologies of the hematopoietic system, such as diseases of a particular blood cell, metabolic disorders, cancers, and autoimmune conditions, among others.

Acute leukemia is provided as just one example of a pathology of the hematopoietic system that can be treated with anti-CD45 antibodies. Acute leukemia is a cancer of blood cells that originates in the bone marrow. According to the National Institutes of Health, there were 26,000 new cases of acute leukemia in the United States in 2022. Conventional first-line treatment for acute leukemia includes chemotherapy and radiation. In many patients, however, hematopoietic cell transplantation (HCT) plays an important role, either as part of an initial curative-intent treatment strategy or as part of a therapeutic strategy to treat relapsed or refractory disease. However, current HCT regimens can cause significant toxicity and adverse events, including graft-versus-host disease and multiorgan dysfunction.

Due to cell surface marker expression in leukemia (and other hematological malignancies), targeted immunotherapies using antibodies against cell surface markers of cancer cells are of particular interest. For instance, 85% to 90% of acute leukemias express CD45. This expression pattern, as well as the stability of expression on the surface of cancer cells renders CD45 a promising target for immunotherapy.

While anti-CD45 antibodies have been identified, a significant concern for antibody-based therapies is the inherent immunogenicity of antibodies derived from rodents. For example, the anti-CD45 murine antibody BC8 recognizes all of the human isoforms of CD45. However, although BC8 is a promising clinical therapeutic, many patients experience significant infusion toxicities because of the murine nature of BC8. Moreover, human anti-mouse antibody (HAMA) immunization can occur, even after just a single BC8 infusion for dosimetry purposes, which precludes future use of any murine mAb in the patient that experienced the HAMA. There is a need for human anti-CD45 antibodies to mitigate immune activation from murine antibody-based therapies.

### SUMMARY OF THE DISCLOSURE

The current disclosure provides chimeric and humanized forms of the BC8 antibody which is a murine IgG1 kappa monoclonal antibody specific for human and nonhuman primate CD45. The disclosed antibodies can be used in the treatment of hematologic malignancies, as lymphodepleting agents prior to adoptive cell therapy, in autologous transplant to restore hematopoietic capability, and/or to facilitate engraftment of normal or gene edited hematopoietic stem/progenitor cells in patients with non-malignant disorders undergoing hematopoietic cell transplantation (HCT), among other uses described elsewhere herein. In particular embodiments, these chimeric or humanized antibodies can be used as research, diagnostic, and/or therapeutic tools against hematologic disorders including malignancies such as acute myeloid leukemia (AML) and acute lymphoblastic leukemia (ALL). The disclosed anti-CD45 antibodies can be used within immunotherapies, such as bi-specific immune-cell engaging constructs, within antibody drug conjugates for chemotherapy and/or radioimmunotherapy and within chimeric antigen receptors (CAR) or engineered T cell receptors (eTCR). The disclosed antibodies can also be provided in multimerized forms. Because the disclosed antibodies are chimeric or humanized, toxicities and HAMA associated with administering a murine antibody are reduced or eliminated.

In particular embodiments, a disclosed chimeric anti-CD45 antibody includes murine variable chain regions and human constant regions. In particular embodiments, a disclosed humanized anti-CD45 antibody includes murine complementarity determining regions (CDRs), human variable chain framework regions, and human constant regions. In particular embodiments, a disclosed humanized anti-CD45 antibody with back mutations includes murine CDRs, human variable chain framework regions, and human constant regions, wherein 1, 2, 3, 4, 5, or 6 residues of the human variable chain region are replaced with murine residues. In certain examples, human constant regions can include Igκ as a light chain constant region and/or IgG1 or IgG4 as a heavy chain constant region. When IgG4 is used as a human heavy chain constant region, the IgG4 can include a S228P mutation.

### BRIEF DESCRIPTION OF THE SEVERAL VIEWS OF THE DRAWINGS

Some of the drawings submitted herewith may be better understood in color. Applicant considers the color versions of the drawings as part of the original submission and reserves the right to present color images of the drawings in later proceedings.

FIGS. 1A, 1B. Comparison of light chain variable domain amino acid sequences of parent murine BC8, CDR-grafted humanized BC8, and CDR-grafted humanized BC8 with back mutations. BC8 light chain variable region (LcFv) is shown (top amino acids) aligned to humanized BC8 (middle amino acids) or humanized BC8 with back mutations (bottom amino acids), using the Clustal alignment format. CDR regions 1-3 are indicated (dashed boxes) as predicted by ABodyBuilder (opig.stats.ox.ac.uk/webapps/newsabdab/sabpred/abodybuilder/) using the method described by North et al. Gaps in alignment are indicated with the character “-”, an asterisk “*” indicates that all 3 sequences have the same nucleotide. Zero dots indicate no conservation, 1 dot indicates some conservation, and 2 dots indicate all pyrimidines or all purines at position.

FIG. 1B. Comparison of heavy chain variable domain amino acid sequences of parent murine BC8, CDR-grafted humanized BC8, and CDR-grafted humanized BC8 with back mutations. BC8 heavy chain variable region (HcFv) is shown (top amino acids) aligned to humanized BC8 (middle amino acids) or humanized BC8 with back mutations (bottom amino acids), using the Clustal alignment format. CDR regions 1-3 are indicated (dashed boxes) as predicted by ABodyBuilder (opig.stats.ox.ac.uk/webapps/newsabdab/sabpred/abodybuilder/) using the method described by North et al. Gaps in alignment are indicated with the character “-”, an asterisk “*” indicates that all 3 sequences have the same nucleotide. Zero dots indicate no conservation, 1 dot indicates some conservation, and 2 dots indicate all pyrimidines or all purines at position.

FIG. 2. Production of chimeric BC8 and humanized BC8. Recombinant chimeric BC8 or humanized BC8 sequences were cloned into pcDNA3.4 vectors containing either human IgG1, human IgG4 S228P, or human IgKappa Fc frameworks, sequence verified, and antibodies expressed using the Expi293 transient transfection system. Antibodies were affinity purified from culture supernatants over HiTrap™ MabSelectSuRe™ columns (Cytiva) using an AKTApurifier chromatography system (Cytiva), and 25 μg of each purified antibody was run on a protein gel under non-reducing (NR) or reducing (R) conditions. Protein gels were stained with a Coomassie blue-based dye to visualize proteins. A molecular weight (MW) marker was included to help identify molecular weights of intact antibodies and reduced antibody chains.

FIG. 3. Competitive immunoreactivity assay. Enzyme-linked immunosorbent assay (ELISA) with the original mouse BC8 mAb and CD45RO ECD peptide was used to test competitive binding of BC8 compared to chimeric BC8 and humanized BC8 mAbs. Plates were coated with CD45RO ECD-mmFc fusion protein and bound to biotinylated BC8 antibody in the presence or absence of increasing amounts of competing unlabeled antibodies, as detected by HRP-Streptavidin.

FIG. 4. Binding of chimeric and humanized BC8 to CD45-positive and CD45-negative cells. Human lymphoid CD45-positive RS4;11 cells (CD45+) were used to derive CD45 knockout cells using CRISPR/Cas9-mediated alteration of the endogenous CD45 locus (CD45−). Staining of CD45+ and CD45− with 5 μg/mL of indicated BC8 antibodies is shown using a PE-conjugated goat anti-human IgG secondary antibody. No staining is observed when using CD45− cells.

FIG. 5. Binding of B10-conjugated humanized BC8 to CD45-positive and CD45-negative cells. Human lymphoid CD45-positive RS4;11 cells (CD45+) and CD45-negative RS4;11 (CD45−) were stained with 10 μg/ml of indicated HuBC8 antibodies and detected using a PE-conjugated goat anti-human IgG secondary antibody. B10 was conjugated to HuBC8 at 5, 10 or 15 equivalents (eq). Data from staining of CD45+ cells with 15 eq B10-HuBC8 IgG4 was lost and not shown. No staining is observed when using CD45− cells.

FIG. 6. Comparison of cell binding of chimeric BC8 versus HuBC8 antibodies consisting of all combinations of CDR-grafted HuBC8 and CDR-grafted HuBC8 with back mutation light and heavy chains. Recombinant versions of HuBC8 antibodies were generated to test the best pairing of humanized BC8 light and heavy chains. (6A and 6B) Human myeloid CD45-positive OCI-AML3 and MOLM-13 cells, and (6C and 6D) human lymphoid RS4;11 cells with CRISPR/Cas9 deletion of CD45 (RS4;11 CD45−) and parental RS4;11 cells (RS4;11 CD45+) were stained with 5 μg/ml of indicated versions of humanized anti-CD45 BC8 antibodies and detected using a PE-conjugated goat anti-human IgG secondary antibody. Data is plotted as an overlay histogram showing relative binding of each antibody to indicated cell lines.

FIG. 7. In vivo CD45+ cell targeting studies with HuBC8 (CDR grafted variant) in immunodeficient mice. In vivo CD45+ cell targeting studies show comparable enrichment of 211At-labeled BC8 and 211At-labeled HuBC8 (CDR grafted variant) relative to appropriate non-binding control antibodies in immunodeficient mice xenotransplanted with human CD45+ leukemia (MOLM-13) cells in the flank. NOD-Rag1null IL2rγnull/J (NRG) mice were chosen for these experiments since they tolerate higher levels of radiation and RIT compared to NOD.SCID/IL2rG−/− (NSG) mice and support transplanted cells from human acute leukemia cell lines without anti-asialo injections to neutralize residual NK cell activity. NRG mice (4 mice per group) were injected subcutaneously with 5×10⁶ MOLM-13 cells in the flank. At day 9, each mouse received 50 μg of antibody that was dual-labelled with 10 μCi At-211 and 5 μCi I-125. BHV-1 (antibody clone 1B8-F11), a monoclonal antibody targeting bovine herpes virus 1, and 13R4, a monoclonal antibody targeting E. coli b-galactosidase that was produced from published sequences (U.S. Pat. No. 8,841,238) as described in Laszlo et al., ([²¹¹At]Astatine-based anti-CD22 radioimmunotherapy for B-cell malignancies. Leuk Lymphoma. 2023; in press) were used as isotype matched controls for murine BC8 (murine IgG1) or HuBC8 (human IgG1). Mice were euthanized and organs harvested at 24 hours. Tissues were analyzed on a gamma counter to calculate the total percent of injected dose/gram of tumor tissue (% ID/g) initially, and again 96 hours to calculate the % ID/g of 1251 in tissues, with the difference representing the 211 At activity. Tumor-to-normal organ ratios were calculated by comparing % ID/g of target tumor to that of uninvolved normal organs.

FIG. 8. In vivo CD45+ cell targeting studies with HuBC8 (CDR grafted variant with back mutations) in immunodeficient mice. In vivo CD45+ cell targeting studies show comparable enrichment of 211At-labeled BC8 (murine IgG1) and human IgG1 and IgG4 (S228P) versions of 211At-labeled HuBC8 (CDR grafted variant with back mutations) in human AML cell tumor tissue relative to appropriate non-binding control antibodies in NRG mice xenotransplanted with human CD45+ leukemia (MOLM-13) cells in the flank. NRG mice (4 mice per group) were injected subcutaneously with 5×10⁶ MOLM-13 cells in the flank. At day 9, each mouse received 50 μg of antibody that was dual-labelled with 10 μCi At-211 and 5 μCi 1-125. BHV-1 (antibody clone 1B8-F11), a monoclonal antibody targeting bovine herpes virus 1, and 13R4, a monoclonal antibody targeting E. coli β-galactosidase, were used as isotype matched controls for murine BC8 and HuBC8. Mice were euthanized and organs harvested at 24 hours. Tissues were analyzed on a gamma counter to calculate the % ID/g initially and again 96 hours to calculate the % ID/g of 1251 in tissues, with the difference representing the 211At activity. Tumor-to-normal organ ratios were calculated by comparing % ID/g of target tumor to that of uninvolved normal organs.

FIG. 9A. In vivo anti-leukemia efficacy studies with HuBC8 (CDR grafted variant) in immunodeficient mice—in vivo fluorescence imaging. In vivo fluorescence imaging 12 and 27 days after administration murine BC8, HuBC8, and appropriate isotype-matched negative control antibodies labeled with 40 μCi of 211At. A control group was left untreated. In this experiment, 0.2×10⁶ luciferase-transduced MOLM-13 cells were injected into the tail veins of NRG mice to generate a disseminated model of human AML. 2 days later, groups of 8 animals were treated with radiolabeled mAbs or left untreated). Another 2 days later, all animals received 5×10⁶ syngeneic bone marrow cells.

FIG. 9B. In vivo anti-leukemia efficacy studies with HuBC8 (CDR grafted variant) in immunodeficient mice—survival analysis. In this experiment, 0.2×10⁶ luciferase-transduced MOLM-13 cells were injected into the tail veins of NRG mice to generate a disseminated model of human AML. 2 days later, groups of 8 animals were treated with radiolabeled mAbs or left untreated). Conditions tested were: murine BC8 labeled with either 20 μCi or 40 μCi of 211At, murine IgG1 isotype control antibody labeled with 40 μCi of 211At, HuBC8 (human IgG1 framework) labeled with either 20 μCi or 40 μCi of 211At, and human IgG1 isotype control antibody labeled with 40 μCi of 211At. Two days after RIT administration, all animals received 5×10⁶ syngeneic bone marrow cells. Mice were then monitored for survival, and any mouse exhibiting excessive toxicity, morbidity, or weight loss euthanized per institutional protocol.

FIG. 10. Sequences supporting the disclosure.

### DETAILED DESCRIPTION

The current disclosure provides chimeric and humanized antibodies that bind CD45. The provided antibodies are chimeric or humanized forms of the BC8 antibody, a murine IgG1 kappa monoclonal antibody specific for human and nonhuman primate CD45. The disclosed chimeric or humanized antibodies can be used as research, diagnostic, and/or therapeutic tools in the treatment of hematologic malignancies, as lymphodepleting agents prior to adoptive cell therapy, in autologous transplant to restore hematopoietic capability, and/or to facilitate engraftment of normal or gene edited hematopoietic stem/progenitor cells in patients with non-malignant disorders undergoing hematopoietic cell transplantation (HCT), among other uses described elsewhere herein. In particular embodiments, the chimeric or humanized antibodies can be used against hematologic disorders including malignancies such as acute myeloid leukemia (AML) and acute lymphoblastic leukemia (ALL). The disclosed anti-CD45 antibodies can be used within immune targeting reagents (e.g., bi-specific antibodies), within antibody drug conjugates (e.g., for chemotherapy and/or radioimmunotherapy) and within chimeric antigen receptors (CAR) or engineered T cell receptors (eTCR) among other uses described elsewhere herein. As is understood by one of ordinary skill in the art, when anti-CD45 antibodies are used within bispecific antibodies or CAR, strategies should be employed (e.g., CD45 knockout) to avoid fratricide as immune effector cells are all CD45+.

The antibodies can also be provided in multimerized forms. The disclosed antibodies reduce or eliminate toxicities and HAMA associated with administering a murine antibody.

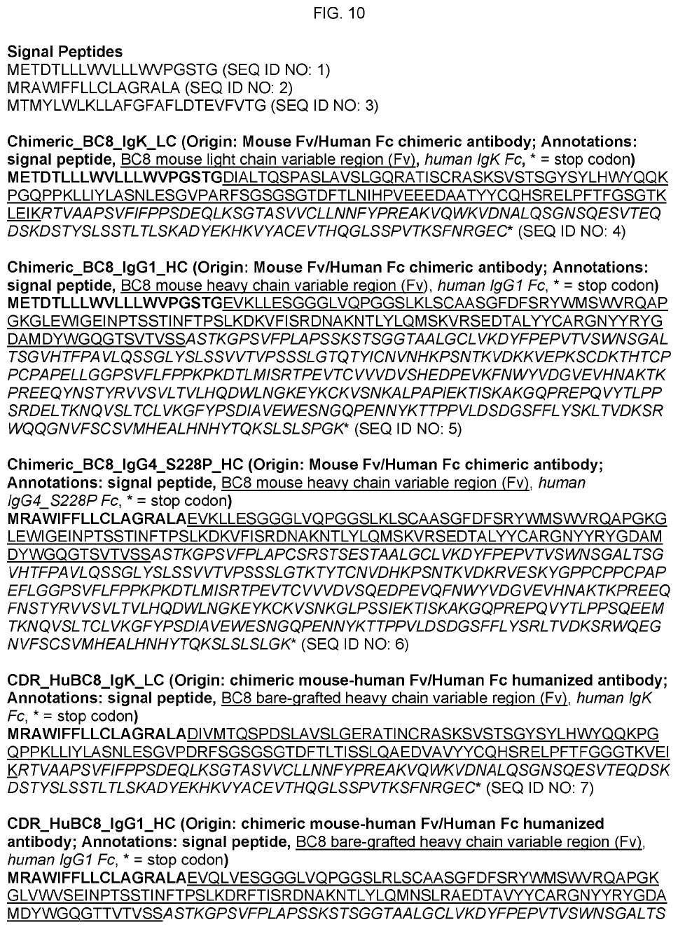

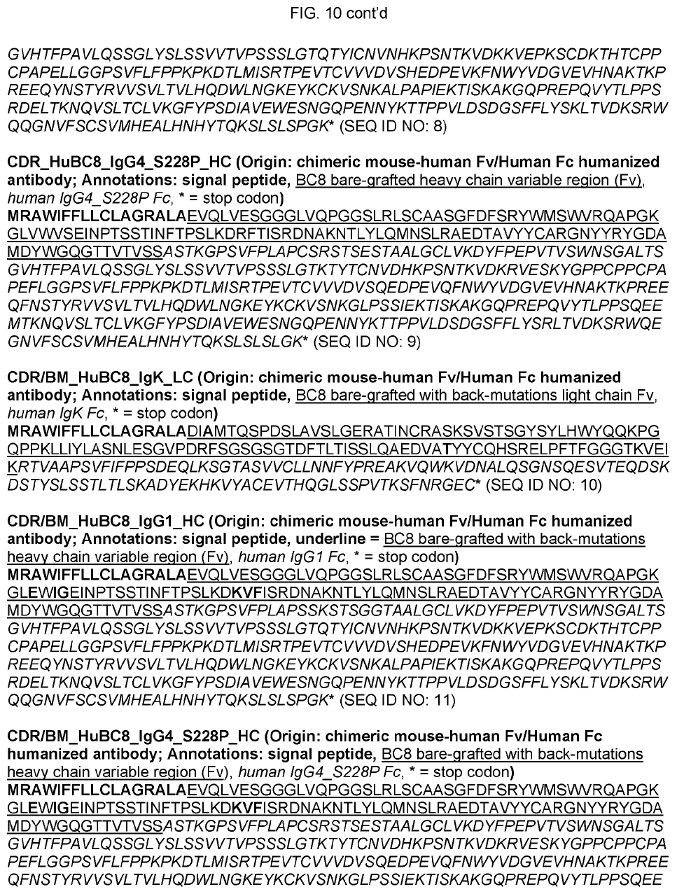

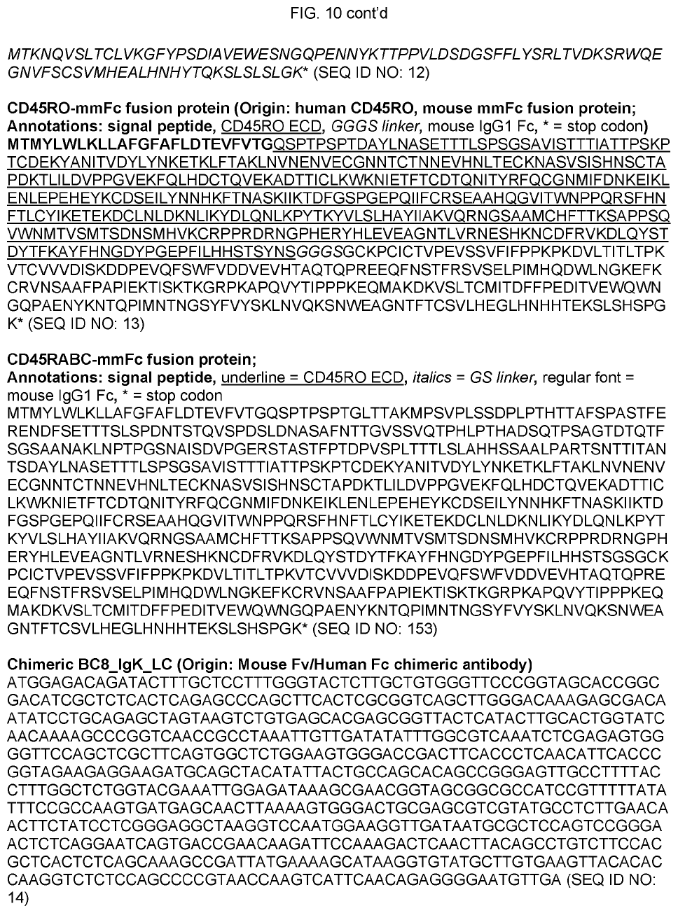

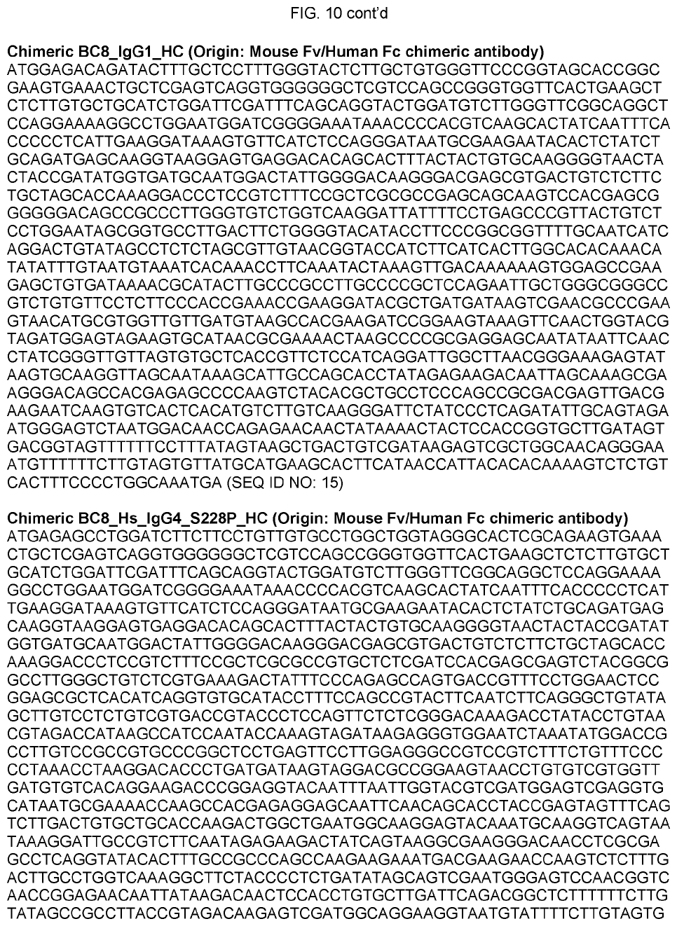

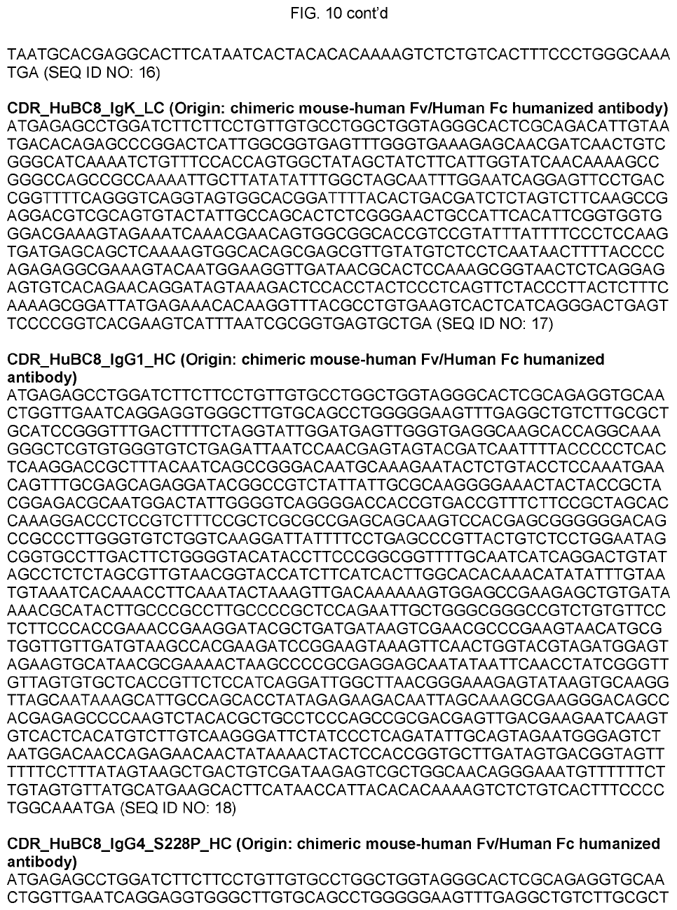

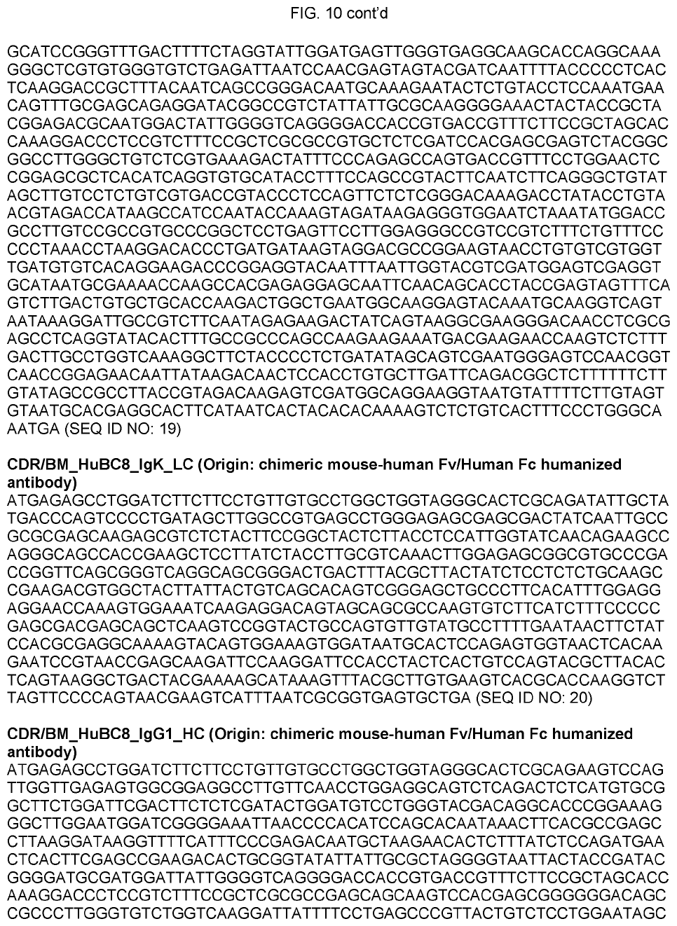

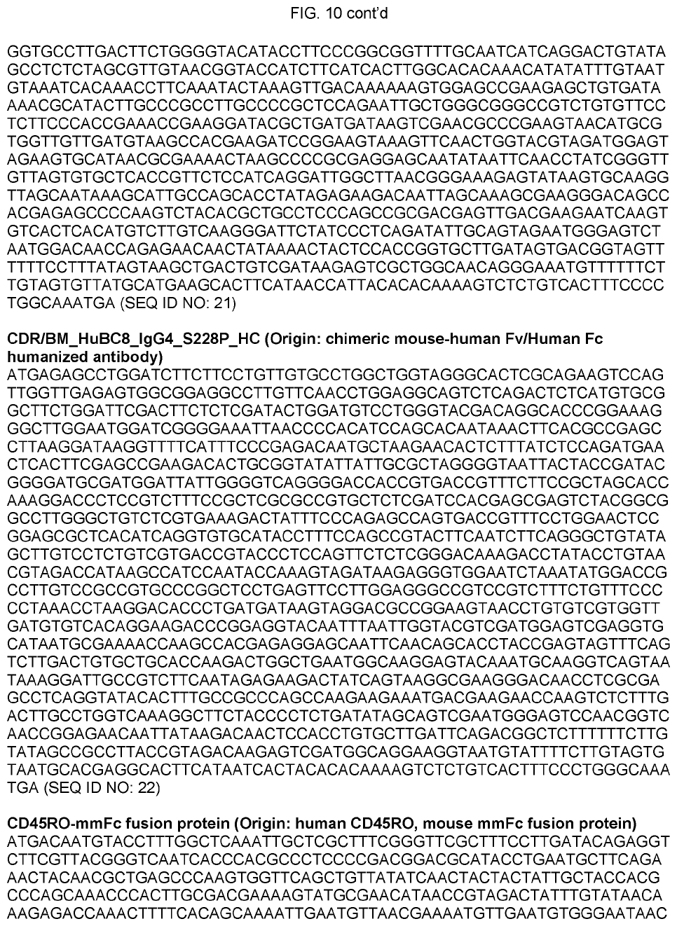

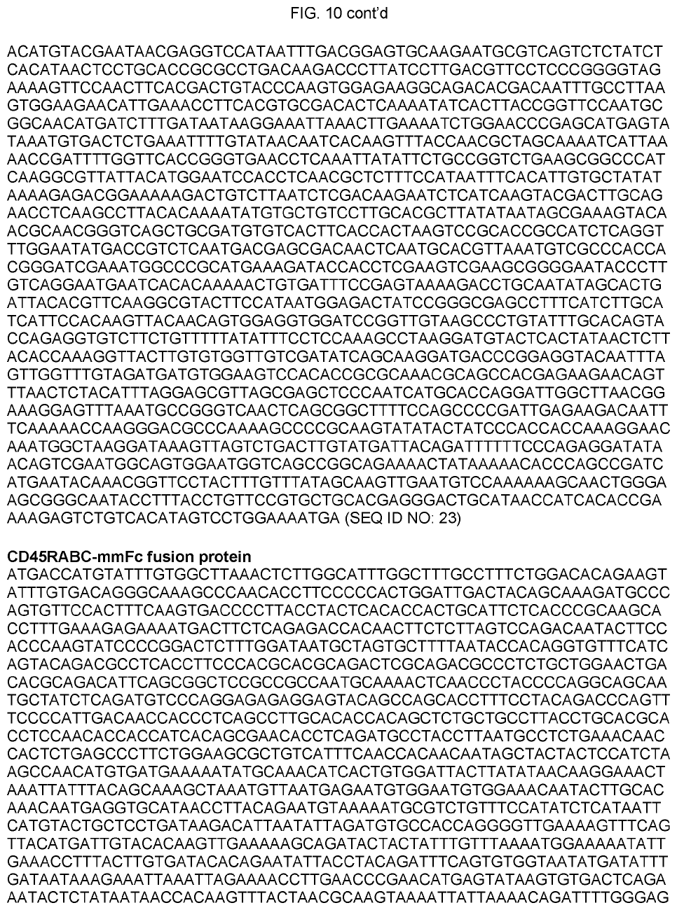

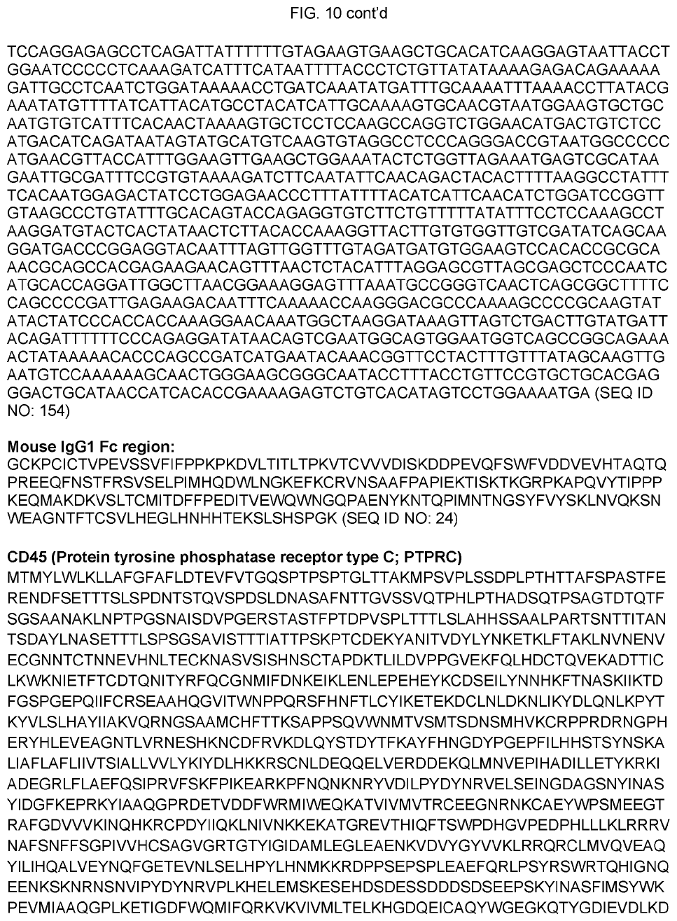

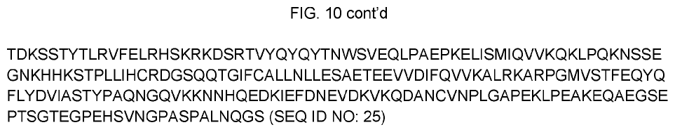

In particular embodiments, a chimeric or humanized antibody that binds CD45 includes a light chain and a heavy chain, wherein the variable region of the light and heavy chain include murine complementarity determining regions (CDR) derived from the BC8 antibody and one or both of (i) a human constant region and (ii) human variable framework regions. In particular embodiments, a disclosed chimeric anti-CD45 antibody includes murine variable chain regions and human constant regions. In particular embodiments, a disclosed humanized anti-CD45 antibody includes murine CDRs, human variable chain framework regions, and human constant regions. In particular embodiments, a disclosed humanized anti-CD45 antibody with back mutations includes murine CDRs, human variable chain framework regions, and human constant chain regions, wherein 1, 2, 3, 4, 5, or 6 residues of the human variable chain region are replaced with murine residues. In particular embodiments, a light chain variable region of the humanized anti-CD45 antibody with back mutations includes murine residues at residue 3 and/or residue 89. In particular embodiments, a heavy chain variable region of the humanized anti-CD45 antibody with back mutations includes murine residues at residues 46, 48, 49, 67, 68, and/or 69. The location of these back mutations are bolded and underlined within the humanized with back mutations light and heavy chain sequences in FIG. 10 (SEQ ID NO: 10, SEQ ID NO: 11, and SEQ ID NO: 12).

In certain examples, human constant regions can include Igκ as a light chain constant region and/or IgG1 or IgG4 as a constant heavy chain. When IgG4 is used as a human heavy chain constant region, the IgG4 can include a S228P mutation.

In particular embodiments, a light chain of an anti-CD45 antibody that includes a murine BC8 light chain variable region and a human Igκ light chain constant region includes a sequence as set forth in SEQ ID NO: 4. This sequence is encoded by the sequence as set forth in SEQ ID NO: 14. In particular embodiments, a heavy chain of an anti-CD45 antibody that includes a murine BC8 heavy chain variable region and a human IgG1 heavy chain constant region includes a sequence as set forth in SEQ ID NO: 5. This sequence is encoded by the sequence as set forth in SEQ ID NO: 15. In particular embodiments, a heavy chain of an anti-CD45 antibody that includes a murine BC8 light chain variable region and a human IgG4_S228P heavy chain constant region includes a sequence as set forth in SEQ ID NO: 6. This sequence is encoded by the sequence as set forth in SEQ ID NO: 16.

In particular embodiments, a light chain of an anti-CD45 antibody that includes a humanized anti-CD45 (CDR HuBC8) light chain variable region and a human Igκ light chain constant region includes a sequence as set forth in SEQ ID NO: 7. This sequence is encoded by the sequence as set forth in SEQ ID NO: 17. In particular embodiments, a heavy chain of an anti-CD45 antibody that includes a humanized anti-CD45 (CDR HuBC8) variable heavy chain and a human IgG1 heavy chain constant region includes a sequence as set forth in SEQ ID NO: 8. This sequence is encoded by the sequence as set forth in SEQ ID NO: 18. In particular embodiments, a heavy chain of an anti-CD45 antibody that includes a humanized anti-CD45 (CDR HuBC8) variable heavy chain and a human IgG4_S228P heavy chain constant region includes a sequence as set forth in SEQ ID NO: 9. This sequence is encoded by the sequence as set forth in SEQ ID NO: 19.

In particular embodiments, a light chain of an anti-CD45 antibody that includes a humanized anti-CD45 with back mutations (CDR/BM HuBC8) light chain variable region and a human Igκ light chain constant region includes a sequence as set forth in SEQ ID NO: 10. This sequence is encoded by the sequence as set forth in SEQ ID NO: 20. In particular embodiments, a heavy chain of an anti-CD45 antibody that includes a CDR/BM HuBC8 heavy chain variable region and a human IgG1 heavy chain constant region includes a sequence as set forth in SEQ ID NO: 11. This sequence is encoded by the sequence as set forth in SEQ ID NO: 21. In particular embodiments, a heavy chain of an anti-CD45 antibody that includes a CDR/BM HuBC8heavy chain variable region and a human IgG4_S228P heavy chain constant region includes a sequence as set forth in SEQ ID NO: 12. This sequence is encoded by the sequence as set forth in SEQ ID NO: 22.

Aspects of the current disclosure are now described in with additional details and options as follows: (I) Antibodies; (II) Chimeric or Humanized Anti-CD45 Antibodies; (Ill) Antibody Variants; (IV) Multi-Domain Binding Molecules; (V) Expression of Recombinant Antibodies; (VI) Anti-CD45 Antibody Conjugates; (VII) Chimeric Antigen Receptors (CARs) and Engineered T Cell Receptors (eTCR); (VIII) Compositions and Formulations; (IX) Methods of Use; (X) Kits; (XI) Exemplary Embodiments; (XII) Experimental Examples; and (XIII) Closing Paragraphs. These headings are provided for organizational purposes only and do not limit the scope or interpretation of the disclosure.

(I) Antibodies. Unless otherwise indicated, an antibody includes a tetramer structure with two full-length heavy chains and two full-length light chains. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4.

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes.

Definitive delineation of a CDR and identification of residues including the binding site of an antibody can be accomplished by solving the structure of the antibody and/or solving the structure of the antibody-epitope complex. In particular embodiments, this can be accomplished by methods such as X-ray crystallography and cryoelectron microscopy. Alternatively, CDRs are determined by comparison to known antibodies (linear sequence) and without resorting to solving a crystal structure. To determine residues involved in binding, a co-crystal structure of the Fab (antibody fragment) bound to the target can optionally be determined. Software programs, such as ABodyBuilder can also be used.

The carboxy-terminal portion of each chain defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g., B-cell receptors); and B-cell activation.

Human light chains are classified as kappa and lambda light chains. In particular embodiments, a human Igκ Fc region includes the sequence: RTVAAPSVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC (SEQ ID NO: 26). In particular embodiments, a human IgA Fc region includes the sequence:

(SEQ ID NO: 27)
GQPKANPTVTLFPPSSEELQANKATLVCLISDFYPGAVTVAWKADGSP
VKAGVETTKPSKQSNNKYAASSYLSLTPEQWKSHRSYSCQVTHEGSTV
EKTVAPTECS.

Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

In particular embodiments, a human IgG1 Fc region includes the sequence:

(SEQ ID NO: 28)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK.

In particular embodiments, a human IgG1 Fc region includes the sequence:

(SEQ ID NO: 29)
THTCPPCPAPEFFGGPSVFFFPPKPKDTFMISRTPEVTCVVVDVSHED
PEVKFNWYVDGVEVHNAKTKPREEQYNSTYRVVSVETVFHQDWENGKE
YKCKVSNKAFPVPIEKTISKAKGQPREPQVYTLPPSREEMTKNQVSLT
CLVKGFYPSDIAVEWESNGQPENNYKTTPPVLDSDGPFFLYSKLTVDK
SRWQQGNVFSCSVMHEALHNHYTQKSLSLSPGK.

In particular embodiments, a human IgG2 Fc region includes the amino acid sequence:

(SEQ ID NO: 30)
ASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDK
TVERKCCVECPPCPAPPVAGPSVFLFPPKPKDTLMISRTPEVTCVVVD
VSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKN
QVSLTCLVKGFYPSDISVEWESNGQPENNYKTTPPMLDSDGSFFLYSK
LTVDKSRWQQGNVFSCSVMHEALHNHYTQKSLSLSPGK

In particular embodiments, a human IgG2 Fc region includes the amino acid sequence:

(SEQ ID NO: 31)
PAPPVAGPSVFLFPPKPKDTLMISRTPEVTCVVVDVSHEDPEVQFNWYV
DGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCS
VMHEALHNHYTQKSLSLSPGK

In particular embodiments, a human IgG3 Fc region includes the amino acid sequence:

(SEQ ID NO: 32)
ASTKGPSVFPLAPCSRSTSGGTAALGCLVKDYFPEPVTVSWNSGALTSG
VHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEP
KSCDTPPPCPRCPAPELLGGPSVFLFPPKPKDTLMISRTPEVTCVVVDV
SHEDPEVQFKWYVDGVEVHNAKTKPREEQYNSTFRVVSVLTVLHQDWLN
GKEYKCKVSNKALPAPIEKTISKTKGQPREPQVYTLPPSREEMTKNQVS
LTCLVKGFYPSDIAVEWESSGQPENNYNTTPPMLDSDGSFFLYSKLTVD
KSRWQQGNIFSCSVMHEALHNRFTQKSLSLSPGK

In particular embodiments, a human IgG3 Fc region includes the amino acid sequence:

(SEQ ID NO: 33)
PAPELLGGPSVFLFPPKPKDTLMISRTPEVTCVVVDVSHEDPEVQFKWY
VDGVEVHNAKTKPREEQFNSTFRVVSVLTVLHQDWLNGKEYKCKVSNKA
LPAPIEKTISKTKGQPREPQVYTLPPSREEMTKNQVSLTCLVKGFYPSD
IAVEWESSGQPENNYNTTPPMLDSDGSFFLYSKLTVDKSRWQQGNIFSC
SVMHEALHNRFTQKSLSLSPGK.

In particular embodiments, a human IgG4 Fc region includes the amino acid sequence:

(SEQ ID NO: 34)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

In particular embodiments, a human IgG4 Fc region includes the amino acid sequence:

(SEQ ID NO: 35)
PAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSC
SVMHEALHNHYTQKSLSLSLGK.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

In particular embodiments, “bind” means that the variable region associates with its target epitope with a dissociation constant (Kd or KD) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷ M, in particular embodiments of from 10⁻⁸ M to 10⁻¹³ M, or in particular embodiments of from 10⁻⁹ M to 10⁻¹³ M. The term can be further used to indicate that the variable region does not bind to other biomolecules present (e.g., it binds to other biomolecules with a dissociation constant (Kd) of 10⁻⁴ M or more, in particular embodiments of from 10⁻⁴ M to 1 M).

In particular embodiments, Kd can be characterized using BIAcore. For example, in particular embodiments, Kd can be measured using surface plasmon resonance assays using a BIACORE®-2000 or a BIACORE®-3000 (BIAcore, Inc., Piscataway, N.J.) at 25° C. with immobilized antigen CM5 chips at 10 response units (RU).

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, human antibodies, bispecific antibodies, trispecific antibodies, tetraspecific antibodies, multi-specific antibodies, polyclonal antibodies, linear antibodies, minibodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup III as in Kabat et al. (supra).

(II) Chimeric or Humanized Anti-CD45 Antibodies. The disclosure provides chimeric or humanized antibodies directed against the CD45 antigen. CD45 refers to any native, mature CD45. CD45, also known as protein tyrosine phosphatase receptor type C (PTPRC) is a type I transmembrane protein present in various isoforms on differentiated hematopoietic cells. In particular embodiments, the antigen CD45 includes the sequence as set forth in SEQ ID NO: 25.

A chimeric antibody is a molecule made up of domains from different species. In a chimeric antibody, at least a portion of the constant region of an antibody of a first species is replaced with at least a portion of the constant region from a second species. In particular embodiments, a chimeric antibody includes a murine variable chain region and a human constant chain region.

In certain examples, a chimeric antibody is an antibody having some or all CDRs and variable region framework sequences entirely or substantially from a non-human “donor” antibody; and constant regions, if present, entirely or substantially from human antibody sequences. Similarly, a chimeric heavy chain has at least one, two and usually all three CDRs and variable heavy chain framework sequence entirely or substantially from a donor antibody heavy chain; and heavy chain constant region, if present, substantially from human heavy chain constant region sequences. Similarly, a chimeric light chain has at least one, two and usually all three CDRs and light chain variable region framework sequence entirely or substantially from a donor antibody light chain; and a light chain constant region, if present, substantially from human light chain constant region sequences. Other than nanobodies and diabodies, a chimeric antibody typically includes a non-human donor heavy chain variable region and a non-human donor light chain variable region. A CDR in a chimeric antibody is substantially from or substantially identical to a corresponding CDR in a non-human antibody with at least 60%, 85%, 90%, 95% or 100% of corresponding residues are identical between the respective CDRs. In particular embodiments, a CDR in a chimeric antibody is substantially from or substantially identical to a corresponding CDR in a non-human antibody when there are no more than 3 conservative amino acid substitutions in each CDR. In certain examples, the variable region framework sequences of an antibody chain are substantially from a non-human donor variable region framework sequence when at least 70%, 80%, 85%, 90%, 95% or 100% of corresponding residues are identical to the non-human variable framework sequence. The constant region of an antibody chain are substantially from a human constant region sequence when at least 70%, 80%, 85%, 90%, 95% or 100% of corresponding residues are identical to the human constant region sequence.

A humanized antibody is an engineered antibody in which the CDRs from a non-human donor antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. In particular embodiments, a humanized antibody includes humanized variable chain regions and human constant regions.

Thus, a humanized antibody is an antibody having some or all CDRs entirely or substantially from a non-human donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a variable heavy chain framework sequence and heavy chain constant region, if present, substantially from human variable heavy chain framework and human heavy chain constant region sequences. Similarly, a humanized light chain has at least one, two and usually all three CDRs entirely or substantially from a donor antibody light chain, and a variable light chain framework sequence and light chain constant region, if present, substantially from human variable light chain framework and human light chain constant region sequences. Other than nanobodies and diabodies, a humanized antibody typically includes a humanized heavy chain and a humanized light chain. A CDR in a humanized or human antibody is substantially from or substantially identical to a corresponding CDR in a non-human antibody with at least 60%, 85%, 90%, 95% or 100% of corresponding residues are identical between the respective CDRs. In particular embodiments, a CDR in a humanized antibody or human antibody is substantially from or substantially identical to a corresponding CDR in a non-human antibody when there are no more than 3 conservative amino acid substitutions in each CDR. The variable region framework sequences of an antibody chain or the constant region of an antibody are substantially from a human variable region framework sequence or human constant region respectively when at least 70%, 80%, 85%, 90%, 95% or 100% of corresponding residues are identical to reference human sequences.

Chimeric and humanized antibodies and methods of making them are reviewed, e.g., in Almagro and Fransson, Front. Biosci. 13:1619-1633, 2008, and are further described, e.g., in Riechmann et al., Nature 332:323-329, 1988; Queen et al., Proc. Nat'l Acad. Sci. USA 86:10029-10033, 1989; U.S. Pat. Nos. 5,821,337, 7,527,791, 6,982,321, and 7,087,409; Kashmiri et al., Methods 36:25-34, 2005 (describing SDR (a-CDR) grafting); Padlan, Mol. Immunol. 28:489-498, 1991 (describing “resurfacing”); Kim, et al., PLoS One 6(5):e19867, 2011 (describing production and characterization of chimeric monoclonal antibodies); Dall'Acqua et al., Methods 36:43-60,2005 (describing “FR shuffling”); and Osbourn et al., Methods 36:61-68, 2005 and Klimka et al., Br. J. Cancer, 83:252-260, 2000 (describing the “guided selection” approach to FR shuffling). EP-B-0239400 provides additional description of “CDR-grafting”, in which one or more CDR sequences of a first antibody is/are placed within a framework of sequences not of that antibody, for instance of another antibody.

In humanized antibodies, certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids. Human framework regions that may be used for humanization include: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al., Proc. Natl. Acad. Sci. USA, 89:4285, 1992; and Presta et al., J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al., J. Biol. Chem. 271:22611-22618, 1996).

The choice of constant region can depend, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 has weak complement-dependent cytotoxicity and human IgG4 lacks complement-dependent cytotoxicity. Human IgG1 and IgG3 also induce stronger cell mediated effector functions than human IgG2 and IgG4.

In particular embodiments, a light chain constant region includes a human Igκ Fc region or a human Igλ Fc region.

In particular embodiments, a heavy chain constant region includes a human IgG1 Fc region or a human IgG4_S228P Fc region.

In particular embodiments, a human IgG4_S228P Fc region includes the sequence:

(SEQ ID NO: 36)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of one or more other isotypes.

Referring to the antibodies provided herein, the following CDR sets are provided. A CDR set refers to 3 light chain CDRs and 3 heavy chain CDRs that together result in binding to CD45.

TABLE 1
Antibody CDR Sequences.

| | CDR | | | SEQ ID |
|---|---|---|---|---|
| Antibody | Definition | CDR | Sequence | NO: |
| BC8, | North | CDRL1 | RASKSVSTSGYSYLH | 37 |
| Chimeric | | CDRL2 | YLASNLES | 38 |
| BC8, | | CDRL3 | QHSRELPFT | 39 |
| CDR | | CDRH1 | AASGFDFSRYWMS | 40 |
| HuBC8, | | CDRH2 | EINPTSSTIN | 41 |
| and CDR/BM | | CDRH3 | ARGNYYRYGDAMDY | 42 |
| HuBC8 | | | | |
| BC8, | IMGT | CDRL1 | KSVSTSGYSY | 43 |
| Chimeric | | CDRL2 | LAS | N/A |
| BC8, | | CDRL3 | QHSRELPFT | 39 |
| CDR | | CDRH1 | GFDFSRYW | 44 |
| HuBC8, and | | CDRH2 | INPTSSTI | 45 |
| CDR/BM | | CDRH3 | ARGNYYRYGDAMDY | 42 |
| HuBC8 | | | | |
| BC8, | Kabat | CDRL1 | RASKSVSTSGYSYLH | 37 |
| Chimeric | | CDRL2 | LASNLES | 46 |
| BC8, | | CDRL3 | QHSRELPFT | 39 |
| CDR | | CDRH1 | GFDFSRYWMS | 47 |
| HuBC8, and | | CDRH2 | EINPTSSTINFTPSLKD | 48 |
| CDR/BM | | CDRH3 | GNYYRYGDAMDY | 49 |
| HuBC8 | | | | |
| BC8, | Chothia | CDRL1 | RASKSVSTSGYSYLH | 37 |
| Chimeric | | CDRL2 | LASNLES | 46 |
| BC8, CDR | | CDRL3 | QHSRELPFT | 39 |
| HuBC8, and | | CDRH1 | GFDFSRY | 50 |
| CDR/BM | | CDRH2 | NPTSST | 51 |
| HuBC8 | | CDRH3 | GNYYRYGDAMDY | 49 |
| BC8, | Contact | CDRL1 | YSYLHWY | 52 |
| Chimeric | | CDRL2 | LLIYLASNLE | 53 |
| BC8, and | | CDRL3 | QHSRELPF | 54 |
| CDR/BM | | CDRH1 | SRYWMS | 55 |
| HuBC8 | | CDRH2 | WIGEINPTSSTIN | 56 |
| | | CDRH3 | ARGNYYRYGDAMD | 57 |
| CDR HuBC8 | Contact | CDRL1 | YSYLHWY | 52 |
| | | CDRL2 | LLIYLASNLE | 53 |
| | | CDRL3 | QHSRELPF | 54 |
| | | CDRH1 | SRYWMS | 55 |
| | | CDRH2 | WVSEINPTSSTIN | 58 |
| | | CDRH3 | ARGNYYRYGDAMD | 57 |

CDR predictions were generated using the program SAbPrep opig.stats.ox.ac.uk/webapps/newsabdab/sabpred/). ABodyBuilder within SAbPred was used (CDR predictions based on “Clothia”).

Although chimeric and humanized antibodies often incorporate all six CDRs from a non-human antibody, they can also be made with less than all CDRs (e.g., at least 3, 4, or 5) CDRs from a non-human antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

In particular embodiments, a chimeric BC8 antibody includes a murine BC8 light chain variable region including the sequence:

(SEQ ID NO: 59)
DIALTQSPASLAVSLGQRATISCRASKSVSTSGYSYLHWYQQKPGQPPK
LLIYLASNLESGVPARFSGSGSGTDFTLNIHPVEEEDAATYYCQHSREL
PFTFGSGTKLEIK.

In particular embodiments, a chimeric BC8 antibody includes a murine BC8 heavy chain variable region including the sequence:

(SEQ ID NO: 60)
EVKLLESGGGLVQPGGSLKLSCAASGFDFSRYWMSWVRQAPGKGLEWIG
EINPTSSTINFTPSLKDKVFISRDNAKNTLYLQMSKVRSEDTALYYCAR
GNYYRYGDAMDYWGQGTSVTVSS.

In particular embodiments, a humanized anti-CD45 antibody includes a CDR HuBC8 light chain variable region including the sequence:

(SEQ ID NO: 61)
DIVMTQSPDSLAVSLGERATINCRASKSVSTSGYSYLHWYQQKPGQPPK
LLIYLASNLESGVPDRFSGSGSGTDFTLTISSLQAEDVAVYYCQHSREL
PFTFGGGTKVEIK.

In particular embodiments, a humanized anti-CD45 antibody includes a CDR HuBC8 heavy chain variable region including the sequence:

(SEQ ID NO: 62)
EVQLVESGGGLVQPGGSLRLSCAASGFDFSRYWMSWVRQAPGKGLVWVS
EINPTSSTINFTPSLKDRFTISRDNAKNTLYLQMNSLRAEDTAVYYCAR
GNYYRYGDAMDYWGQGTTVTVSS.

In particular embodiments, a humanized anti-CD45 antibody includes a CDR/BM HuBC8 light chain variable region including the sequence:

(SEQ ID NO: 63)
DIAMTQSPDSLAVSLGERATINCRASKSVSTSGYSYLHWYQQKPGQPPK
LLIYLASNLESGVPDRFSGSGSGTDFTLTISSLQAEDVATYYCQHSREL
PFTFGGGTKVEIK.

particular embodiments, a humanized anti-CD45 antibody includes a CDR/BM HuBC8 heavy chain variable region including the sequence:

(SEQ ID NO: 64)
EVQLVESGGGLVQPGGSLRLSCAASGFDFSRYWMSWVRQAPGKGLEWIG
EINPTSSTINFTPSLKDKVFISRDNAKNTLYLQMNSLRAEDTAVYYCAR
GNYYRYGDAMDYWGQGTTVTVSS.

In particular embodiments, an anti-CD45 antibody includes a light chain variable region selected from: a murine BC8 light chain variable region, a CDR HuBC8 light chain variable region, or a CDR/BM HuBC8 light chain variable region; and a heavy chain variable region selected from: a murine BC8 heavy chain variable region, a CDR HuBC8 heavy chain variable region, or a CDR/BM HuBC8 heavy chain variable region. In particular embodiments, a chimeric anti-CD45 antibody includes a murine BC8 light chain variable region and a murine BC8 heavy chain variable region. In particular embodiments, a humanized anti-CD45 antibody includes a CDR HuBC8 light chain variable region and a CDR HuBC8 heavy chain variable region. In particular embodiments, a humanized anti-CD45 antibody includes a CDR/BM HuBC8 light chain variable region and a CDR/BM HuBC8 heavy chain variable region. In particular embodiments, an anti-CD45 antibody includes a CDR/BM HuBC8 light chain variable region and a CDR HuBC8 heavy chain variable region. In particular embodiments, a humanized anti-CD45 antibody includes a CDR HuBC8 light chain variable region and a CDR/BM HuBC8 heavy chain variable region.

In particular embodiments, an anti-CD45 antibody includes a light chain including a murine BC8 light chain variable region and a Igκ light chain constant region; and a heavy chain including a murine BC8 heavy chain variable region and a IgG1 heavy chain constant region.

In particular embodiments, an anti-CD45 antibody includes a light chain including a murine BC8 light chain variable region and a Igκ light chain constant region; and a heavy chain including a murine BC8 heavy chain variable region and a IgG4_S228P heavy chain constant region.

In particular embodiments, an anti-CD45 antibody includes a light chain including a CDR HuBC8 light chain variable region and a Igκ light chain constant region; and a heavy chain including a CDR HuBC8 heavy chain variable region and a IgG1 heavy chain constant region.

In particular embodiments, an anti-CD45 antibody includes a light chain including a CDR HuBC8 light chain variable region and a Igκ light chain constant region; and a heavy chain including a CDR HuBC8 heavy chain variable region and a IgG4_S228P heavy chain constant region.

In particular embodiments, an anti-CD45 antibody includes a light chain including a CDR/BM HuBC8 light chain variable region and a Igκ light chain constant region; and a heavy chain including a CDR/BM HuBC8 heavy chain variable region and a IgG1 heavy chain constant region.

In particular embodiments, an anti-CD45 antibody includes a light chain including a CDR/BM HuBC8 light chain variable region and a Igκ light chain constant region; and a heavy chain including a CDR/BM HuBC8 heavy chain variable region and a IgG4_S228P heavy chain constant region.

In particular embodiments, an anti-CD45 antibody includes a light chain including a CDR/BM HuBC8 light chain variable region and a Igκ light chain constant region; and a heavy chain including a CDR HuBC8 heavy chain variable region and a IgG1 heavy chain constant region.

In particular embodiments, an anti-CD45 antibody includes a light chain including a CDR/BM HuBC8 light chain variable region and a Igκ light chain constant region; and a heavy chain including a CDR HuBC8 heavy chain variable region and a IgG4_S228P heavy chain constant region.

In particular embodiments, an anti-CD45 antibody includes a light chain including a CDR HuBC8 light chain variable region and a Igκ light chain constant region; and a heavy chain including a CDR/BM HuBC8 heavy chain variable region and a IgG1 heavy chain constant region.

In particular embodiments, an anti-CD45 antibody includes a light chain including a CDR HuBC8 light chain variable region and a Igκ light chain constant region; and a heavy chain including a CDR/BM HuBC8 heavy chain variable region and a IgG4_S228P heavy chain constant region.

(III) Antibody Variants. Antibodies disclosed herein can be utilized to prepare various forms of relevant binding domain molecules. For example, particular embodiments can include binding fragments of an antibody, e.g., Fv, Fab, Fab′, F(ab′)₂, and single chain Fv fragments (scFvs) or any biologically effective fragments of an immunoglobulin that bind specifically to an epitope described herein.

In particular embodiments, an antibody fragment is used. An “antibody fragment” denotes a portion of a full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894; and 5,587,458.

Linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. Linker sequences of scFv are commonly Gly-Ser linkers, described in more detail elsewhere herein.

Additional examples of antibody-based binding domain formats include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Antibody fragments can also include isolated CDRs. For a review of antibody fragments, see Hudson, et al., Nat. Med. 9:129-134, 2003.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions. Numerous Fc modifications are known in the art, and a representative sampling of such possible modifications are described herein.

In particular embodiments, variants (including Fc variants) have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-live. While the disclosure below describes these modifications in terms of their application to antibodies, when applicable to another particular anti-CD45 binding domain format (e.g., bispecific antibodies), the modifications can also be applied to these other formats.

In particular embodiments, an IgG4 Fc region is mutated to form the IgG4_S228P Fc region. IgG4 antibodies can undergo a process called Fab arm exchange which results in functionally monovalent, bispecific antibodies with unknown specificity and thus potentially reduced therapeutic efficacy. Mutating the wildtype IgG4 serine at position 228 within the core-hinge region to a proline creates the IgG4_S228P mutant. In particular embodiments, the IgG4_S228P mutant prevents Fab arm exchange. In particular embodiments, the S228P mutation is located at residue 108 of SEQ ID NO: 36.

In particular embodiments the antibodies can be mutated to increase their affinity for Fc receptors. Exemplary mutations that increase the affinity for Fc receptors include: G236A/S239D/A330L/1332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164: 4178-4184, 2000.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, the antibody can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations. In particular embodiments, Fc polypeptide amino acid alterations can include M252Y, S254T, T256E, M428L, and/or N434S and can be used together, separately or in any combination. For example, M428L/N434S is a pair of mutations that increase the half-life of antibodies in serum, as described in Zalevsky et al., Nature Biotechnology 28, 157-159, 2010. Other alterations that can be helpful are described in U.S. Pat. Nos. 7,083,784, 7,670,600, US Publication No. 2010/0234575, PCT/US2012/070146, and Zwolak, Scientific Reports 7: 15521, 2017. In particular embodiments, any substitution at one of the following amino acid positions in an Fc polypeptide can be considered an Fc alteration that extends half-life: 250, 251, 252, 259, 307, 308, 332, 378, 380, 428, 430, 434, 436. Each of these alterations or combinations of these alterations can be used to extend the half-life of a bispecific antibody as described herein.

In particular embodiments, Fc modifications include huIgG4 ProAlaAla, huIgG2m4, and/or huIgG2sigma mutations. In particular embodiments, one or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). For additional information regarding Fc mutations that create administration benefits, see Saunders, Conceptual Approaches to Modulating Antibody Effector Functions and Circulation Half-Life, Frontiers in Immunology (2019) Vol. 10, Article 1296.

(IV) Multi-Domain Binding Molecules. Multi-domain binding molecules include at least two binding domains, wherein at least one binding domain includes an anti-CD45 binding domain disclosed herein. In particular embodiments, a multi-domain binding molecule includes at least one, at least two, at least, three, at least four binding domains that bind an epitope on CD45. In particular embodiments, all of the binding domains of a multi-domain binding molecule bind CD45.

Multi-domain binding molecules include bispecific antibodies which bind at least two epitopes wherein at least one of the epitopes is located on CD45. Multi-domain binding molecules include trispecific antibodies which binds at least 3 epitopes, wherein at least one of the epitopes is located on CD45, and so on.

Bispecific antibodies can be prepared utilizing antibody fragments (for example, F(ab′)₂ bispecific antibodies). For example, WO 1996/016673 describes a bispecific anti-ErbB2/anti-Fc gamma RIII antibody; U.S. Pat. No. 5,837,234 describes a bispecific anti-ErbB2/anti-Fc gamma RI antibody; WO 1998/002463 describes a bispecific anti-ErbB2/Fc alpha antibody; and U.S. Pat. No. 5,821,337 describes a bispecific anti-ErbB2/anti-CD3 antibody.

Some additional exemplary bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, as indicated, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

Two antibodies or fragments thereof can be linked through a linker to form a bispecific antibody. In particular embodiments, the two antibodies or fragments thereof can bind the same epitope or different epitopes. Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target.

Commonly used flexible linkers include linker sequence with the amino acids glycine and serine (Gly-Ser linkers). In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 65), (Gly₃Ser)ₙ(Gly₄Ser)ₙ (SEQ ID NO: 66), (Gly₃Ser)ₙ(Gly₂Ser)ₙ (SEQ ID NO: 67), and (Gly₃Ser)ₙ(Gly₄Ser)₁ (SEQ ID NO: 68). In particular embodiments, the linker is (Gly₄Ser)₄ (SEQ ID NO: 69), (Gly₄Ser)₃ (SEQ ID NO: 70), (Gly₄Ser)₂ (SEQ ID NO: 71), (Gly₄Ser)₁ (SEQ ID NO: 72), (Gly₃Ser)₂ (SEQ ID NO: 73), (Gly₃Ser)₁ (SEQ ID NO: 74), (Gly₂Ser)₂ (SEQ ID NO: 75) or (Gly₂Ser)₁, GGSGGGSGGSG (SEQ ID NO: 76), GGSGGGSGSG (SEQ ID NO: 77), or GGSGGGSG (SEQ ID NO: 78).

Linkers that include one or more antibody hinge regions and/or immunoglobulin heavy chain constant regions, such as CH3 alone or a CH2CH3 sequence can also be used. Additional examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

In particular embodiments, binding domains disclosed herein can be used to create bi-, tri, (or more) specific immune cell engaging molecules. Immune cell engaging molecules have at least one binding domain that binds a receptor on an immune cell and alters the activation state of the immune cell. Examples of multi-domain immune cell engaging molecules include those which bind both an immune cell (e.g., T-cell or NK-cells) activating epitope and CD45, with the goal of bringing immune cells to CD45-expressing cells to destroy them. See, for example, US 2008/0145362. Such molecules are referred to herein as immune-activating multi-specifics or I-AMS). BiTEs® (Amgen, Thousand Oaks, CA) are one form of I-AMS. Immune cells that can be targeted for localized activation by I-AMS within the current disclosure include, for example, B-cells, T-cells, natural killer (NK) cells, and macrophages which are discussed in more detail herein.

I-AMS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1BB (CD137), OX40, lymphocyte function-associated antigen-1 (LFA-1), LIGHT, NKG2C, and B7-H3.

In particular embodiments, the CD3 binding domain (e.g., scFv) is derived from the OKT3 antibody (the same as the one utilized in blinatumomab), otelixizumab, teplizumab, visilizumab, 20G6-F3, 4B4-D7, 4E7-C9, 18F5-H10, or TR66. The OKT3 antibody is described in detail in U.S. Pat. No. 5,929,212.

In particular embodiments, the OKT3 binding domain includes a light chain variable region of QIVLTQSPAIMSASPGEKVTMTCSASSSVSYMNWYQQKSGTSPKRWIYDTSKLASGVPAHFR GSGSGTSYSLTISGMEAEDAATYYCQQWSSNPFTFGSGTKLEINR (SEQ ID NO: 79) and a heavy chain variable region of QVQLQQSGAELARPGASVKMSCKASGYTFTRYTMHWVKQRPGQGLEWIGYINPSRGYTNYN QKFKDKATLTTDKSSSTAYMQLSSLTSEDSAVYYCARYYDDHYCLDYWGQGTTLTVSSAKTTA PSVYPLAPVCGDTTGSSVTLGCLVKGYFPEPVTLTWNSGSLSSGVHTFPAVLQSDLYTLSSSV TVTSS (SEQ ID NO: 80).

In particular embodiments, the binding domain includes a light chain variable region including a CDRL1 sequence including SASSSVSYMN (SEQ ID NO: 81), a CDRL2 sequence including DTSKLAS (SEQ ID NO: 82), a CDRL3 sequence including QQWSSNPFTF (SEQ ID NO: 83), a CDRH1 sequence including RYTMH (SEQ ID NO: 84), a CDRH2 sequence including YINPSRGYTNYNQKFKD (SEQ ID NO: 85), and a CDRH3 sequence including YYDDHYCL (SEQ ID NO: 86). In particular embodiments, the binding domain is human or humanized. For more information regarding binding domains that bind CD3, see U.S. Pat. No. 8,785,604, PCT/US 17/42264, and/or WO02051871.

In particular embodiments, a binding domain is “derived from” a reference antibody when the binding domain includes the CDRs of the reference antibody, according to a known numbering scheme (e.g., Kabat, Chothia, Martin, or others).

CD28 binds to B7-1 (CD80) and B7-2 (CD86) and is the most potent of the known co-stimulatory molecules (June et al., Immunol. Today 15:321, 1994; Linsley et al., Ann. Rev. Immunol. 11:191, 1993). In particular embodiments, the CD28 binding domain is derived from TGN1412, CD80, CD86 or the 9D7 antibody. Additional antibodies that bind CD28 include 9.3, KOLT-2, 15E8, 248.23.2, and EX5.3D10.

In particular embodiments, the binding domain that binds CD28 is derived from TGN-1412 and/or theralizumab. In particular embodiments, the binding domain includes a light chain variable region of DIQMTQSPSSLSASVGDRVTITCKTNENIYSNLAWYQQKDGKSPQLLIYAATHLVEGVPSRFSG SGSGTQYSLTISSLQPEDFGNYYCQHFWGTPXTFGGGTKLEI KR, wherein X=C, A, or N. (SEQ ID NO: 87) and a heavy chain variable region of VQLQQSGAELKKPGASVKVSCKASGYTFTEYIIHWIKLRSGQGLEWIGWFYPGSNDIQYNAQF KGKATLTADKSSSTVYMELTGLTPEDSAVYFCARRDDFSGYDALPYWGQGTLVTVSA (SEQ ID NO: 88). In particular embodiments, the binding domain includes a light chain variable region including a CDRL1 sequence including HASQNIYVWLN (SEQ ID NO: 89), a CDRL2 sequence including KASNLHT (SEQ ID NO: 90), a CDRL3 sequence including QQGQTYPYT (SEQ ID NO: 91), a CDRH1 sequence including SYYIH (SEQ ID NO: 92), a CDRH2 sequence including CIYPGNVNTNYNEKFKD (SEQ ID NO: 93), and a CDRH3 sequence including SHYGLDWNFDV (SEQ ID NO: 94). In particular embodiments, the binding domain is human or humanized. For more information regarding binding domains that bind CD28, see U.S. Pat. No. 8,785,604 and/or WO02051871.

Activated T-cells express 4-1BB (CD137). In particular embodiments, the 4-1BB binding domain includes a light chain variable region including a CDRL1 sequence including RASQSVS (SEQ ID NO: 95), a CDRL2 sequence including ASNRAT (SEQ ID NO: 96), and a CDRL3 sequence including QRSNWPPALT (SEQ ID NO: 97) and a heavy chain variable region including a CDRH1 sequence including YYWS (SEQ ID NO: 98), a CDRH2 sequence including INH, and a CDRH3 sequence including YGPGNYDWYFDL (SEQ ID NO: 99).

Particular embodiments disclosed herein including binding domains that bind epitopes on CD8. In particular embodiments, the CD8 binding domain (e.g., scFv) is derived from the OKT8 antibody.

In particular embodiments natural killer cells (also known as NK-cells, K-cells, and killer cells) are targeted for localized activation by I-AMS. NK cells can induce apoptosis or cell lysis by releasing granules that disrupt cellular membranes and can secrete cytokines to recruit other immune cells.

Examples of commercially available antibodies that bind to an NK cell receptor and induce and/or enhance activation of NK cells include: 5C6 and 1D11, which bind and activate NKG2D (available from BioLegend® San Diego, CA); mAb 33, which binds and activates KIR2DL4 (available from BioLegend®); P44-8, which binds and activates NKp44 (available from BioLegend®); SK1, which binds and activates CD8; and 3G8 which binds and activates CD16.

Binding domains of I-AMS and other engineered formats described herein may be joined through a linker. As indicated previously, a linker is an amino acid sequence which can provide flexibility and room for conformational movement between the binding domains of a I-AM. Any appropriate linker may be used.

Other forms of bispecific binding molecules include the single chain “Janusins” described in Traunecker et al. (Embo Journal, 10, 3655-3659, 1991).

Bispecific binding molecules with extended half-lives are described in, for example, U.S. Pat. No. 8,921,528 and US Patent Publication No. 2014/0308285.

Because albumin has an extended serum half-life, it can be of use in improving the pharmacokinetics of administered anti-CD45 antibodies. In particular embodiments, anti-CD45 antibodies can be linked to albumin. In other particular embodiments, anti-CD45 antibodies can be linked to albumin-binding domains (ABDs). ABDs include, for example, albumin-binding peptides, antibodies, antibody fragments, and designed ankyrin repeat proteins (DARPins).

In particular embodiments, multi-domain binding molecules with extended half-lives include multi-domain binding molecules wherein at least one binding domain binds albumin. In particular embodiments, the multi-domain binding molecule that binds albumin includes a binding domain that binds CD45 linked to a binding domain that binds albumin.

In particular embodiments, an albumin-binding domain has the sequence: DITGAALLEAKEAAINELKQYGISDYYVTLINKAKTVEGVNALKAEILSALP (SEQ ID NO: 100). In particular embodiments, an albumin-binding domain includes a variant of the sequence as set forth in SEQ ID NO: 100, wherein the variant sequence is modified by at least one amino acid substitution selected from the group including: E12D, T29H-K35D, and A45D.

In particular embodiments, an albumin-binding domain includes the sequence: LKEAKEKAIEELKKAGITSDYYFDLINKAKTVEGVNALKDEILKA (SEQ ID NO: 101). In particular embodiments, an albumin-binding domain includes a variant of the sequence as set forth in SEQ ID NO: 101, wherein the variant sequence is modified by at least one amino acid substitution selected from the group including: Y21, Y22, L25, K30, T31, E33, G34, A37, L38, E41, 142 and A45.

Additional binding domains that bind albumin include CA645 as described in Adams et al., 2016 MAbs 8(7): 1336-1346 (see, e.g., Protein Data Bank accession codes 5FUZ and 5FUO); anti-HSA Nanobody™ (Ablynx, Ghent, Belgium), AlbudAb™ (GlaxoSmithKline, Brentford, United Kingdom), and other high-affinity albumin nanobody sequences as described in Shen et al., 2020 bioRxiv doi://doi.org/10.1101/2020.08.19.257725; Mester, et al., 2021 mAbs. 13:1; Tijink et al., 2008 Mol Cancer Ther (7) (8) 2288-2297; and Roovers et al., Cancer Immunol Immunother 2007; 56: 303-317.

In particular embodiments, multi-domain binding molecules are multimers of an antibody disclosed herein. Multimerization strategies include formation of a fusion protein using protein linkers or use of IgA or IgM constant regions as a multimerization scaffold. In certain aspects, multimerization is achieved by linking antibodies or binding domains of antibodies in a fusion protein with protein linkers. Fusion proteins include different protein domains linked to each other directly or through intervening linker segments such that the function of each included domain is retained.

Certain examples include fusion protein with two or three copies of an antibody or binding domain disclosed herein (e.g., chimeric or humanized anti-CD45 antibody), each linked with the Gly-Ser linker (Gly₄Ser)ₙ (SEQ ID NO: 65) wherein n is an integer of 1, 2, 3, 4, 5, 6, 7, 8, 9 or 10. In particular embodiments, n is 3.

A “multimerization domain” is a domain that causes two or more proteins (monomers) to interact with each other through covalent and/or non-covalent association(s). Multimerization domains are highly conserved protein sequences that can include different types of sequence motifs such as leucine zipper, helix loop-helix, ankyrin and PAS (Feuerstein et al, Proc. Natl. Acad. Sci. USA, 91:10655-10659, 1994). Multimerization domains present in proteins can bind to form dimers, trimers, tetramers, pentamers, hexamers, heptamers, etc., depending on the number of units/monomers incorporated into the multimer, and/or homomultimers or heteromultimers, depending on whether the binding monomers are the same type or a different type (U.S. patent Ser. No. 10/030,065).

Dimerization domains can include protein sequence motifs such as coiled coils, acid patches, zinc fingers, calcium hands, a CH1-CL pair, an “interface” with an engineered “knob” and/or “protruberance” (U.S. Pat. No. 5,821,333), leucine zippers (U.S. Pat. No. 5,932,448), SH2 and SH3 (Vidal et al., Biochemistry, 43:7336-44, 2004), PTB (Zhou et al., Nature, 378:584-592, 1995), WW (Sudol Prog Biochys MoL Bio, 65:113-132, 1996), PDZ (Kim et al., Nature, 378: 85-88, 1995; Komau et al., Science, 269:1737-1740, 1995) and WD40 (Hu et al., J Biol Chem., 273:33489-33494, 1998). Additional examples of molecules that contain dimerization domains/motifs are receptor dimer pairs such as the interleukin-8 receptor (IL-8R), integrin heterodimers such as LFA-I and GPIIIb/IIIa, dimeric ligand polypeptides such as nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa et al., J Biol. Chem., 269:27833-27839, 1994; Radziejewski et al., Biochem, 32: 1350, 1993) and variants of some of these domains with modified affinities (PCT Publication No. WO 2012/001647).

In particular embodiments, the sequence corresponding to a dimerization motif/domain includes the leucine zipper domain of Jun (U.S. Pat. No. 5,932,448; RIARLEEKVKTLKAQNSELASTANMLREQVAQLKQKVMN (SEQ ID NO: 102)), the dimerization domain of Fos (U.S. Pat. No. 5,932,448; LTDTLQAETDQLEDKKSALQTEIANLLKEKEKLEFILAA (SEQ ID NO: 103)), a consensus sequence for a WW motif (PCT Publication No. WO 1997/037223), the dimerization domain of the SH2B adapter protein from GenBank Accession no. AAF73912.1 (Nishi et al., Mol Cell Biol, 25: 2607-2621, 2005; WREFCESHARAAALDFARRFRLYLASHPQYAGPGAEAAFSRRFAELFLQHFEAEVARAS (SEQ ID NO: 104)), the SH3 domain of I1l from GenBank Accession no. AAD22543.1 (Kristensen el al., EMBO J., 25: 785-797, 2006; THRAIFRFVPRHEDELELEVDDPLLVELQAEDYWYEAYNMRTGARGVFPAYYAIE (SEQ ID. NO: 105)), the PTB domain of human DOK-7 from GenBank Accession no. NP_005535.1 (Wagner et al., Cold Spring Harb Perspect Biol. 5: a008987, 2013; LGEVHRFHVTVAPGTKLESGPATLHLCNDVLVLARDIPPAVTGQWKLSDLRRYGAVPSGFIFEG GTRCGYWAGVFFLSSAEGEQISFLFDCIVRGISPTKG (SEQ ID NO: 106)), the PDZ-like domain of SATB1 from UniProt Accession No. Q01826 (Galande et al., Mol Cell Biol. August; 21: 5591-5604, 2001; DCKEEHAEFVLVRKDMLFNQLIEMALLSLGYSHSSAAQAKGLIQVGKWNPVPLSYVTDAPDAT VADMLQDVYHVVTLKIQLHSCPKLEDLPPEQWSHTTVRNALKDLLKDMNQSS (SEQ ID NO: 107)), the WD40 repeats of APAF from UniProt Accession No. 014727 (Jorgensen et al., 2009. PLOS One. 4(12):e8463; CAPWPMVEKLIKQCLKENPQERPTSAQVFDILNSAELVCLTRRILLPKNVIVECMVATHHNSRN ASIWLGCGHTDRGQLSFLDLNTEGYTSEEVADSRILCLALVHLPVEKESWIVSGTQSGTLLVINT EDGKKRHTLEKMTDSVTCLYCNSFSKQSKQKNFLLVGTADGKLAIFEDKTVKLKGAAPLKILNIG NVSTPLMCLSESTNSTERNVMWGGCGSQLFSYAAFSDSNIITVVVDTALYIAKQNSPVVEVWD KKTEKLCGLIDCVHFLREVMVKETKIFSFSNDFTIQKLIETRTNKESKHKMSYSGRVKTLCLQKN TALWIGTGGGHILLLDLSTRRLIRVIYNFCNSVRVMMTAQLGSLKNVMLVLGYNRKNTEGTQKQ KEIQSCLTVWDINLPHEVQNLEKHIEVRKELAEKMRRTSVE (SEQ ID NO: 108)), the PAS motif of the dioxin receptor from UniProt Accession No. 16L9E7 (Pongratz et al., Mol Cell Biol, 18:4079-4088, 1998; DQELKHLILEAADGFLFIVSCETGRVVYVSDSVTPVLNQQQSEWFGSTLYDQVHPDDVDKLRE QLSTSENALTGR (SEQ ID NO: 109)) and the EF hand motif of parvalbumin from UniProt Accession No. P20472 (Jamalian et al., Int J Proteomics, 2014: 153712, 2014;

(SEQ ID NO: 110))
LSAKETKMLMAAGDKDGDGKIGVDEFSTLVAES.

In particular embodiments, the dimerization domain can be a dimerization and docking domain (DDD) on one antibody and an anchoring domain (AD) on another antibody to facilitate a stably tethered structure. In particular embodiments, the DDD (DDD1 and DDD2) are derived from the regulatory subunits of a cAMP-dependent protein kinase (PKA), and the AD (AD1 and AD2) are derived from a specific region found in various A-kinase anchoring proteins (AKAPs) that mediates association with the R subunits of PKA. In particular embodiments, DDD1 includes the amino acid sequence: SHIQIPPGLTELLQGYTVEVLRQQPPDLVEFAVEYFTRLREARA (SEQ ID NO: 111). In particular embodiments, DDD2 includes the amino acid sequence: CGHIQIPPGLTELLQGYTVEVLRQQPPDLVEFAVEYFTRLREARA (SEQ ID NO: 112). In particular embodiments, AD1 includes the amino acid sequence: QIEYLAKQIVDNAIQQA (SEQ ID NO: 113). In particular embodiments, AD2 includes the amino acid sequence: CGQIEYLAKQIVDNAIQQAGC (SEQ ID NO: 114). However, one skilled in the art will realize that other DDDs and ADs are known and can be used such as: the 4-helix bundle type DDD domains may be obtained from p53, DCoH (pterin 4 alpha carbinolamine dehydratase/dimerization cofactor of hepatocyte nuclear factor 1 alpha (TCF1)) and HNF-1 (hepatocyte nuclear factor 1). Other AD sequences of potential use may be found in Patent Publication No. US2003/0232420A1.

The X-type four-helix bundle dimerization motif that is a structural characteristic of the DDD (Newlon, et al. EMBO J. 2001; 20: 1651-1662; Newlon, et al. Nature Struct Biol. 1999; 3: 222-227) is found in other classes of proteins, such as the S100 proteins (for example, S100B and calcyclin), and the hepatocyte nuclear factor (HNF) family of transcriptional factors (for example, HNF-1a and HNF-1p). Over 300 proteins that are involved in either signal transduction or transcriptional activation also contain a module of 65-70 amino acids termed the sterile a motif (SAM) domain, which has a variation of the X-type four-helix bundle present on its dimerization interface. For S100B, this X-type four-helix bundle enables the binding of each dimer to two p53 peptides derived from the c-terminal regulatory domain (residues 367-388) with micromolar affinity (Rustandi, et al. Biochemistry. 1998; 37: 1951-1960). Similarly, the N-terminal dimerization domain of HNF-1a (HNF-p1) was shown to associate with a dimer of DCoH (dimerization cofactor for HNF-1) via a dimer of HNF-p1 (Rose, et al. Nature Struct Biol. 2000; 7: 744-748). In alternative embodiments, these naturally occurring systems can also be used to provide stable multimeric structures with multiple functions or binding specificities. Other binding events such as those between an enzyme and its substrate/inhibitor, for example, cutinase and phosphonates (Hodneland, et al. Proc Natl Acd Sci USA. 2002; 99: 5048-5052), may also be utilized to generate the two associating components (the “docking” step), which are subsequently stabilized covalently (the “lock” step).

In particular embodiments, dimerization of antibodies can be induced by a chemical inducer. This method of dimerization requires one antibody to contain a chemical inducer of dimerization binding domain 1 (CBD1) and the second antibody to contain the second chemical inducer of dimerization binding domain (CBD2), wherein CBD1 and CBD2 are capable of simultaneously binding to a chemical inducer of dimerization (CID). If the CID is rapamycin, CBD1 and CBD2 can be the rapamycin binding domain of FK-binding protein 12 (FKBP12) and the FKBP12-Rapamycin Binding (FRB) domain of mTOR. In particular embodiments, FKBP12 includes the sequence:

(SEQ ID NO: 115)
MGVQVETISPGDGRTFPKRGQTCWHYTGMLEDGKKFDSSRDRNPFKFML
GKQEVIRGWEEGVAQMSVGQRAKLTISPDYAYGATGHPGIIPPHATLVF
DVELLKLE.

In particular embodiments, FRB includes the sequence: MASRILWHEMWHEGLEEASRLYFGERNVKGMFEVLEPLHAMMERGPQTLKETSFNQAYGRD LMEAQEWCRKYMKSGNVKDLTQAWDLYYHVFRRISKLES (SEQ ID NO: 116). If the CID is FK506/cyclosporin fusion protein or a derivative thereof, CBD1 and CBD2 can be the FK506 (Tacrolimus) binding domain of FK-binding protein 12 (FKBP12) and the cyclosporin binding domain of cylcophilin A. If the CID is estrone/biotin fusion protein or a derivative thereof, CBD1 and CBD2 can be an oestrogen-binding domain (EBD) and a streptavidin binding domain. If the CID is dexamethasone/methotrexate fusion molecule or a derivative thereof, CBD1 and CBD2 can be a glucocorticoid-binding domain (GBD) and a dihydrofolate reductase (DHFR) binding domain. If the CID is O⁶-benzylguanine derivative/methotrexate fusion molecule or a derivative thereof, CBD1 and CBD2 can be an O⁶-alkylguanine-DNA alkyltransferase (AGT) binding domain and a dihydrofolate reductase (DHFR) binding domain. If the CID is RSL1 or a derivative thereof, CBD1 and CBD2 can be a retinoic acid receptor domain and an ecodysone receptor domain. If the CID is AP1903 or a derivative thereof, CBD1 and CBD2 can be the FK506 binding protein (FKBP12) binding domains including a F36V mutation. Use of the CID binding domains can also be used to alter the affinity to the CID. For instance, altering amino acids at positions 2095, 2098, and 2101 of FRB can alter binding to Rapamycin: KTW has high, KHF intermediate and PLW is low (Bayle et al, Chemistry & Biology 13, 99-107, January 2006).

In particular embodiments, antibodies can multimerize using a transmembrane polypeptide derived from a FcεRI chain. In particular embodiments, an antibody can include a part of a FcεRI alpha chain and another antibody can include a part of an FcεRI beta chain or variant thereof such that said FcεRI chains spontaneously dimerize together to form a dimeric antibody. In particular embodiments, antibodies can include a part of a FcεRI alpha chain and a part of a FcεRI gamma chain or variant thereof such that said FcεRI chains spontaneously trimerize together to form a trimeric antibody, and in another embodiment the multi-chain antibody can include a part of FcεRI alpha chain, a part of FcεRI beta chain and a part of FcεRI gamma chain or variants thereof such that said FcεRI chains spontaneously tetramerize together to form a tetrameric antibody.

In particular embodiments, additional methods of causing dimerization can be utilized. Additional modifications to generate a dimerization domain in antibody could include: replacing the C-terminus domain with murine counterparts; generating a second interchain disulfide bond in the C-terminus domain by introducing a second cysteine residue into both antibodies; swapping interacting residues in each of the antibodies in the C-terminus domains (“knob-in-hole”); and fusing the variable domains of the antibodies directly to CD3ζ (CD3ζ fusion) (Schmitt et al., Hum. Gene Ther. 2009. 20:1240-1248).

Particular embodiments can utilize multimerization domains, such as C4b multimerization domains or ferritin multimerization domains. Full-length native C4b includes seven α-chains linked together by a multimerization (i.e., heptamerization) domain at the C-terminus of the α-chains. Blom et al., (2004) Mol Immunol 40: 1333-1346. Ferritin is an iron storage protein found in almost all living organisms, and has been extensively studied and engineered for a number of biochemical/biomedical purposes (US 20090233377; Meldrum, et al. Science 257, 522-523 (1992); U.S. 20110038025; Yamashita, Biochim Biophys Acta 1800, 846-857 (2010), including as a multimerizing vaccine platform for displaying peptide epitopes (US 20060251679 (2006); Li, et al. Industrial Biotechnol 2, 143-147 (2006)).

Mutlimerization with encapsulin and lumazine synthase can also be performed. Both can be linked to antibodies to create self-assembling 60mer particles (Jardine et al., 2013, Science 340, 711-716 and Kanekiyo et al., 2015, Cell 162, 1090-1100).

Multimerized antibodies and antibody-like molecules such as IgA and IgM antibodies have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134, 10,400,038, and 9,938,347, U.S. Patent Application Publication Nos. US20190100597A1, US20180118814A1, US20180118816A1, US20190185570A1, and US20180265596A1, and PCT Publication Nos. WO 2018/017888, WO 2018/017763, WO 2018/017889, WO 2018/017761, and WO 2019/165340.

Particular embodiments include using IgA and IgM constant region domains to allow the binding portion of molecules provided herein to readily multimerize into dimers, pentamers or hexamers. Basic immunoglobulin structures in vertebrate systems are described above and are well understood. (See, e.g., Harlow et al., Antibodies: A Laboratory Manual, Cold Spring Harbor Laboratory Press, 2nd ed. 1988).

Immunoglobulin A (IgA), as the major class of antibody present in the mucosal secretions of most mammals, represents a key first line of defense against invasion by inhaled and ingested pathogens. IgA is also found at significant concentrations in the serum of many species, where it functions as a second line of defense mediating elimination of pathogens that have breached the mucosal surface. Receptors specific for the Fc region of IgA, FcaR, are key mediators of IgA effector function. Native IgA is a tetrameric protein including two identical light chains (K or A) and two identical heavy chains. IgA, similarly to IgG, contains three constant domains (CA1-CA3), with a hinge region between the CA1 and CA2 domains. The main difference between IgA1 and IgA2 resides in the hinge region that lies between the two Fab arms and the Fc region. IgA1 has an extended hinge region due to the insertion of a duplicated stretch of amino acids, which is absent in IgA2. Both forms of IgA have the capacity to form dimers, in which two monomer units, are arranged in an end-to-end configuration stabilized by disulfide bridges and incorporation of a J-chain. J-chains are also part of IgM pentamers and are discussed in more detail below.

Both IgA and IgM (discussed further below in relation to pentamers and hexamers) possess an 18-amino acid extension in the C terminus called the “tail-piece” (tp). The IgA and IgM tp is highly conserved among various animal species. The conserved penultimate cysteine residue in the IgA and IgM tp has been demonstrated to be involved in multimerization by forming a disulfide bond between heavy chains to permit formation of a multimer. Both tp contain an N-linked carbohydrate addition site, the presence of which is required for dimer formation in IgA and J-chain incorporation and pentamer formation in IgM. However, the structure and composition of the N-linked carbohydrates in the tp differ, suggesting differences in the accessibility of the glycans to processing by glycosyltransferases. Particularly, the IgA (atp) and IgM (μtp) tp differ at seven amino acid positions.

The human IgA1 constant region typically includes the amino acid sequence: ASPTSPKVFPLSLCSTQPDGNVVIACLVQGFFPQEPLSVTWSESGQGVTARNFPPSQDASGDL YTTSSQLTLPATQCLAGKSVTCHVKHYTNPSQDVTVPCPVPSTPPTPSPSTPPTPSPSCCHPR LSLHRPALEDLLLGSEANLTCTLTGLRDASGVTFTWTPSSGKSAVQGPPERDLCGCYSVSSVL PGCAEPWNHGKTFTCTAAYPESKTPLTATLSKSGNTFRPEVHLLPPPSEELALNELVTLTCLAR GFSPKDVLVRWLQGSQELPREKYLTWASRQEPSQGTTTFAVTSILRVAAEDWKKGDTFSCMV GHEALPLAFTQKTIDRLAGKPTHVNVSVVMAEVDGTCY (SEQ ID NO: 117). Referring to this SEQ ID NO: 117, the human CA1 domain extends from amino acid 6 to amino acid 98; the human IgA1 hinge region extends from amino acid 102 to amino acid 124, the human CA2 domain extends from amino acid 125 to amino acid 219, the human CA3 domain extends from amino acid 228 to amino acid 330, and the tp extends from amino acid 331 to amino acid 352.

The human IgA2 constant region typically includes the amino acid sequence ASPTSPKVFPLSLDSTPQDGNVVVACLVQGFFPQEPLSVTWSESGQNVTARNFPPSQDASGD LYTTSSQLTLPATQCPDGKSVTCHVKHYTNPSQDVTVPCPVPPPPPCCHPRLSLHRPALEDLL LGSEANLTCTLTGLRDASGATFTWTPSSGKSAVQGPPERDLCGCYSVSSVLPGCAQPWNHG ETFTCTAAHPELKTPLTANITKSGNTFRPEVHLLPPPSEELALNELVTLTCLARGFSPKDVLVRW LQGSQELPREKYLTWASRQEPSQGTTTFAVTSILRVAAEDWKKGDTFSCMVGHEALPLAFTQK TIDRLAGKPTHVNVSVVMAEVDGTCY (SEQ ID NO: 118). Referring to this SEQ ID NO: 118, the human CA1 domain extends from amino acid 6 to amino acid 98, the human IgA2 hinge region extends from amino acid 102 to amino acid 111, the human CA2 domain extends from amino acid 113 to amino acid 206, the human CA3 domain extends from amino acid 215 to amino acid 317, and the tp extends from amino acid 318 to amino acid 340.

As indicated, two IgA binding units can form a complex with two additional polypeptide chains, the J chain (e.g., SEQ ID NO: 119, the mature human J chain) and the secretory component to form a bivalent secretory IgA (sIgA)-derived binding molecule. An exemplary precursor secretory component includes the sequence MLLFVLTCLLAVFPAISTKSPIFGPEEVNSVEGNSVSITCYYPPTSVNRHTRKYWCRQGARGGC ITLISSEGYVSSKYAGRANLTNFPENGTFVVNIAQLSQDDSGRYKCGLGINSRGLSFDVSLEVS QGPGLLNDTKVYTVDLGRTVTINCPFKTENAQKRKSLYKQIGLYPVLVIDSSGYVNPNYTGRIRL DIQGTGQLLFSVVINQLRLSDAGQYLCQAGDDSNSNKKNADLQVLKPEPELVYEDLRGSVTFH CALGPEVANVAKFLCRQSSGENCDVVVNTLGKRAPAFEGRILLNPQDKDGSFSVVITGLRKED AGRYLCGAHSDGQLQEGSPIQAWQLFVNEESTIPRSPTVVKGVAGGSVAVLCPYNRKESKSIK YWCLWEGAQNGRCPLLVDSEGWVKAQYEGRLSLLEEPGNGTFTVILNQLTSRDAGFYWCLTN GDTLWRTTVEIKIIEGEPNLKVPGNVTAVLGETLKVPCHFPCKFSSYEKYWCKWNNTGCQALP SQDEGPSKAFVNCDENSRLVSLTLNLVTRADEGWYWCGVKQGHFYGETAAVYVAVEERKAA GSRDVSLAKADAAPDEKVLDSGFREIENKAIQDPRLFAEEKAVADTRDQADGSRASVDSGSSE EQGGSSRALVSTLVPLGLVLAVGAVAVGVARARHRKNVDRVSIRSYRTDISMSDFENSREFGA NDNMGASSITQETSLGGKEEFVATTESTTETKEPKKAKRSSKEEAEMAYKDFLLQSSTVAAEA QDGPQEA (SEQ ID NO: 120). An exemplary mature secretory component includes KSPIFGPEEVNSVEGNSVSITCYYPPTSVNRHTRKYWCRQGARGGCITLISSEGYVSSKYAGR ANLTNFPENGTFVVNIAQLSQDDSGRYKCGLGINSRGLSFDVSLEVSQGPGLLNDTKVYTVDL GRTVTINCPFKTENAQKRKSLYKQIGLYPVLVIDSSGYVNPNYTGRIRLDIQGTGQLLFSVVINQL RLSDAGQYLCQAGDDSNSNKKNADLQVLKPEPELVYEDLRGSVTFHCALGPEVANVAKFLCR QSSGENCDVVVNTLGKRAPAFEGRILLNPQDKDGSFSVVITGLRKEDAGRYLCGAHSDGQLQE GSPIQAWQLFVNEESTIPRSPTVVKGVAGGSVAVLCPYNRKESKSIKYWCLWEGAQNGRCPLL VDSEGWVKAQYEGRLSLLEEPGNGTFTVILNQLTSRDAGFYWCLTNGDTLWRTTVEIKIIEGEP NLKVPGNVTAVLGETLKVPCHFPCKFSSYEKYWCKWNNTGCQALPSQDEGPSKAFVNCDEN SRLVSLTLNLVTRADEGWYWCGVKQGHFYGETAAVYVAVEERKAAGSRDVSLAKADAAPDEK VLDSGFREIENKAIQDPR (SEQ ID NO: 121). While not wishing to be bound by theory, and as indicated above, the assembly of two IgA binding units into a dimeric IgA-derived binding molecule is thought to involve the CA3 and tp domains. See, e.g., Braathen, R., el al., J. Biol. Chem. 277:42755-42762 (2002). Accordingly, a multimerizing dimeric IgA-derived binding molecule provided in this disclosure typically includes IgA constant regions that include at least the CA3 and tp domains.

An engineered IgA heavy chain constant region can additionally include a CA2 domain or a fragment thereof, an IgA hinge region or fragment thereof, a CA1 domain or a fragment thereof, and/or other IgA (or other immunoglobulin, e.g., IgG) heavy chain domains, including, e.g., an IgG hinge region. In certain embodiments, a binding molecule as provided herein can include a complete IgA heavy chain constant region (e.g., SEQ ID NO: 117 or SEQ ID NO: 118), or a variant, derivative, or analog thereof.

In particular embodiments, the IgA heavy chain constant regions can include amino acids 125 to 353 of SEQ ID NO: 117 or amino acids 113 to 340 of SEQ ID NO: 118. In particular embodiments, the IgA heavy chain constant regions can each further include an IgA or IgG hinge region situated N-terminal to the IgA CA2 domains. For example, the IgA heavy chain constant regions can include amino acids 102 to 353 of SEQ ID NO: 117 or amino acids 102 to 340 of SEQ ID NO: 118. In particular embodiments, the IgA heavy chain constant regions can each further include an IgA CA1 domain situated N-terminal to the IgA hinge region.

Each of the strategies discussed above can be used to create IgA antibody-based dimers.

Particular embodiments include IgM immunoglobulin constant region domains that allow the binding portion of molecules provided herein to readily multimerize into pentamers or hexamers.

Particular embodiments include IgM constant regions (or variants thereof). These embodiments have the ability to form hexamers, or in association with a J-chain, form pentamers. Embodiments with an IgM constant region typically include at least the Cμ4-tp domains of the IgM constant region but can include heavy chain constant region domains from other antibody isotypes, e.g., IgG, from the same species or from a different species. In particular embodiments, one or more constant region domains can be deleted so long as the IgM antibody is capable of forming hexamers and/or pentamers. Thus, an IgM antibody can be, e.g., a hybrid IgM/IgG antibody or can be a “multimerizing fragment” of an IgM-derived binding molecule.

The assembly of five or six IgM binding units into a pentameric or hexameric IgM antibody is thought to involve the Cμ4 and tp domains. See, e.g., Braathen, R., et al., J Biol. Chem. 277:42755-42762 (2002). Accordingly, a pentameric or hexameric IgM antibody described in this disclosure typically includes at least the Cμ4 and/or tp domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains.

Five IgM monomers form a complex with a J-chain to form a native IgM molecule. The J-chain is considered to facilitate polymerization of p chains before IgM is secreted from antibody-producing cells. Sequences for the human IGJ gene are known in the art, for example, (IGMT Accession: J00256, X86355, M25625, AJ879487). The J chain establishes the disulfide bridges between IgM antibodies to form multimeric structures such as pentamers. See, for example, Sorensen et al. International Immunology, (2000), pages 19-27. While crystallization of IgM has proved to be notoriously challenging, Czajkowsky and Shao (PNAS 106(35): 14960-14965, 2009) published a homology-based structural model of IgM, based on the structure of the IgE Fc domain and the known disulfide pairings. The authors report that the human IgM pentamer is a mushroom-shaped molecule with a flexural bias. The IgM heavy (p) chain contains five N-linked glycosylation sites: Asn-171, Asn-332, Asn-395, Asn-402 and Asn-563. In an IgM antibody where each binding unit is bivalent, the binding molecule itself can have 10 or 12 valencies.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, b-2 Microglobulins, Major Histocompatibility Antigens, Thy-I, Complement, C-Reactive Protein, Thymopoietin, Integrins, Post-gamma Globulin, a-2 Macroglobulins, and Other Related Proteins,” U.S. Dept of Health and Human Services (1991). IgM constant regions can be numbered sequentially (i.e., amino acid #1 starting with the first amino acid of the constant region) or by using the Kabat numbering scheme.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tp, as indicated above.

In particular embodiments, each binding unit of a multimeric binding molecule as provided herein includes two IgM heavy chain constant regions or multimerizing fragments or variants thereof, each including at least an IgM Cμ4 domain and an IgM tp domain. In certain embodiments the IgM heavy chain constant regions can each further include an IgM Cμ3 domain situated N-terminal to the IgM Cμ4 and IgM tp domains.

In particular embodiments, the IgM heavy chain constant regions can each further include an IgM Cμ2 domain situated N-terminal to the IgM Cμ3 domain. Exemplary multimeric binding molecules provided herein include human IgM constant regions that include the wild-type human Cμ2, Cμ3, and Cμ4-tp domains as follows:

(SEQ ID NO: 122)
VIAELPPKVSVFVPPRDGFFGNPRKSKLICQATGFSPRQIQVSWLREGK
QVGSGVTTDQVQAEAKESGPTTYKVTSTLTIKESDWLSQSMFTCRVDHR
GLTFQQNASSMCVPDQDTAIRVFAIPPSFASIFLTKSTKLTCLVTDLTT
YDSVTISWTRQNGEAVKTHTNISESHPNATFSAVGEASICEDDWNSGER
FTCTVTHTDLPSPLKQTISRPKGVALHRPDVYLLPPAREQLNLRESATI
TCLVTGFSPADVFVQWMQRGQPLSPEKYVTSAPMPEPQAPGRYFAHSIL
TVSEEEWNTGETYTCVVAHEALPNRVTERTVDKSTGKPTLYNVSLVMSD
TAGTCY.

In certain IgM-derived multimeric binding molecules as provided herein each IgM constant region can include, instead of, or in addition to an IgM Cμ2 domain, an IgG hinge region or functional variant thereof situated N-terminal to the IgM Cμ3 domain. An exemplary variant human IgG1 hinge region amino acid sequence in which the cysteine at position 6 is substituted with serine is VEPKSSDKTHTCPPCPAP (SEQ ID NO: 123). An exemplary IgM constant region of this type includes the variant human IgG1 hinge region fused to a multimerizing fragment of the human IgM constant region including the Cμ3, Cμ4, and tp domains, and includes the amino acid sequence:

(SEQ ID NO: 124)
VEPKSSDKTHTCPPCPAPDQDTAIRVFAIPPSFASIFLTKSTKLTCLV
TDLTTYDSVTISWTRQNGEAVKTHTNISESHPNATFSAVGEASICEDD
WNSGERFTCTVTHTDLPSPLKQTISRPKGVALHRPDVYLLPPAREQLN
LRESATITCLVTGFSPADVFVQWMQRGQPLSPEKYVTSAPMPEPQAPG
RYFAHSILTVSEEEWNTGETYTCVVAHEALPNRVTERTVDKSTGKPTL
YNVSLVMSDTAGTCY.

Human IgM constant regions, and also certain non-human primate IgM constant regions, as provided herein typically include five (5) naturally-occurring asparagine (N)-linked glycosylation motifs or sites. As used herein “an N-linked glycosylation motif” includes the amino acid sequence N-X1-S/T, wherein N is asparagine, X1 is any amino acid except proline (P), and S/T is serine (S) or threonine (T). The glycan is attached to the nitrogen atom of the asparagine residue. See, e.g., Drickamer K, Taylor ME (2006), Introduction to Glycobiology (2nd ed.). Oxford University Press, USA. N-linked glycosylation motifs occur in the human IgM heavy chain constant regions of SEQ ID NO: 125 or SEQ ID NO: 126 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Each of these sites in the human IgM heavy chain constant region, except for N4, can be mutated to prevent glycosylation at that site, while still allowing IgM expression and assembly into a hexamer or pentamer.

The human IgM heavy chain constant region typically includes the amino acid sequence GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL SQSMFTCRVDHRGLTFQQNASSMCVPDQDTAIRVFAIPPSFASIFLTKSTKLTCLVTDLTTYDSV TISWTRQNGEAVKTHTNISESHPNATFSAVGEASICEDDWNSGERFTCTVTHTDLPSPLKQTIS RPKGVALHRPDVYLLPPAREQLNLRESATITCLVTGFSPADVFVQWMQRGQPLSPEKYVTSAP MPEPQAPGRYFAHSILTVSEEEWNTGETYTCVVAHEALPNRVTERTVDKSTGKPTLYNVSLVM SDTAGTCY (SEQ ID NO: 125; identical to, e.g., GenBank Accession Nos. pir∥S37768, CAA47708.1, and CAA47714.1). Referring to this SEQ ID NO: 125, the human Cμ1 region ranges from amino acid 5 to amino acid 102; the human Cμ2 region ranges from amino acid 114 to amino acid 205, the human Cμ3 region ranges from amino acid 224 to amino acid 319, the Cμ4 region ranges from amino acid 329 to amino acid 430, and the tp ranges from amino acid 431 to amino acid 453.

In particular embodiments, an IgM heavy chain constant region includes the sequence: GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL GQSMFTCRVDHRGLTFQQNASSMCVPDQDTAIRVFAIPPSFASIFLTKSTKLTCLVTDLTTYDS VTISWTRQNGEAVKTHTNISESHPNATFSAVGEASICEDDWNSGERFTCTVTHTDLPSPLKQTI SRPKGVALHRPDVYLLPPAREQLNLRESATITCLVTGFSPADVFVQWMQRGQPLSPEKYVTSA PMPEPQAPGRYFAHSILTVSEEEWNTGETYTCVVAHEALPNRVTERTVDKSTGKPTLYNVSLV MSDTAGTCY (SEQ ID NO: 126; (UniProt ID P01871)-allele IGHM*04). This sequence differs from SEQ ID NO: 125 by one amino acid at position 191.

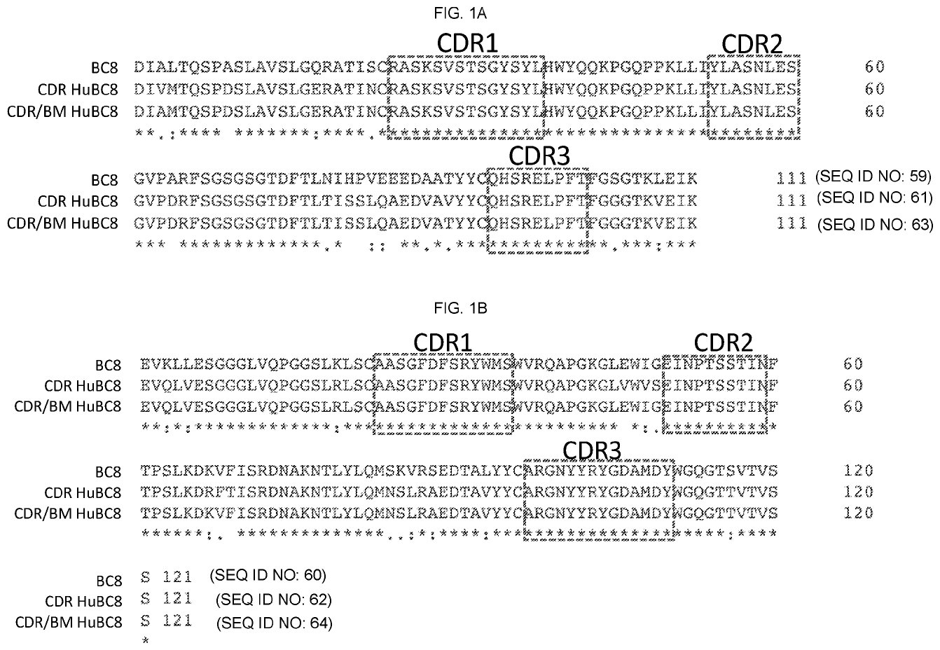

Other forms of the human IgM constant region with minor sequence variations exist, including GenBank Accession Nos. P01871.4, CAB37838.1, and pir∥MHHU. The amino acid substitutions, insertions, and/or deletions at positions corresponding to SEQ ID NO: 125 described herein can likewise be incorporated into alternate human IgM sequences, as well as into IgM constant region amino acid sequences of other species, e.g., those shown in FIG. 1 of PCT/US2019/020374.

In certain aspects, a variant human IgM constant region includes an amino acid substitution corresponding to the wild-type human IgM constant region at position P311, P313, R344, E345, S401, E402, and/or E403 of SEQ ID NO: 125. These positions correspond to the Kabat numbering system as follows: S401 of SEQ ID NO: 125 corresponds to S524 of Kabat; E402 of SEQ ID NO: 125 corresponds to E525 of Kabat; E403 of SEQ ID NO: 125 corresponds to E526 of Kabat; R344 of SEQ ID NO: 125 corresponds to R467 of Kabat; and E345 of SEQ ID NO: 125 corresponds to E468 of Kabat.

In particular embodiments, “corresponds to” means the designated position of SEQ ID NO: 125 and the amino acid in the sequence of the IgM constant region of any species which is homologous to the specified position. See FIG. 1 of PCT/US2019/020374.

In particular embodiments, P311 of SEQ ID NO: 125 can be substituted, e.g., with alanine (P311A), serine (P311S), or glycine (P311G) and/or P313 of SEQ ID NO: 125 can be substituted, e.g., with alanine (P313A), serine (P313S), or glycine (P313G). P311 and P313 of SEQ ID NO: 125 can be substituted with alanine (P311A) and serine (P313S), respectively as shown in the following sequence:

(SEQ ID NO: 127)
GSASAPTLFPLVSCENSPSDTSSVAVGCLAQDFLPDSITFSWKYKNNS
DISSTRGFPSVLRGGKYAATSQVLLPSKDVMQGTDEHVVCKVQHPNGN
KEKNVPLPVIAELPPKVSVFVPPRDGFFGNPRKSKLICQATGFSPRQI
QVSWLREGKQVGSGVTTDQVQAEAKESGPTTYKVTSTLTIKESDWLSQ
SMFTCRVDHRGLTFQQNASSMCVPDQDTAIRVFAIPPSFASIFLTKST
KLTCLVTDLTTYDSVTISWTRQNGEAVKTHTNISESHPNATFSAVGEA
SICEDDWNSGERFTCTVTHTDLASSLKQTISRPKGVALHRPDVYLLPP
AREQLNLRESATITCLVTGFSPADVFVQWMQRGQPLSPEKYVTSAPMP
EPQAPGRYFAHSILTVSEEEWNTGETYTCVVAHEALPNRVTERTVDKS
TGKPTLYNVSLVMSDTAGTCY.
(mutations in bold underline)

In certain aspects, S401 of SEQ ID NO: 125 can be substituted with any amino acid. In certain aspects, S401 of SEQ ID NO: 125 can be substituted with alanine (A) as follows (alanine substitution indicated by bold underline):

(SEQ ID NO: 128)
GSASAPTLFPLVSCENSPSDTSSVAVGCLAQDFLPDSITFSWKYKNNS
DISSTRGFPSVLRGGKYAATSQVLLPSKDVMQGTDEHVVCKVQHPNGN
KEKNVPLPVIAELPPKVSVFVPPRDGFFGNPRKSKLICQATGFSPRQI
QVSWLREGKQVGSGVTTDQVQAEAKESGPTTYKVTSTLTIKESDWLSQ
SMFTCRVDHRGLTFQQNASSMCVPDQDTAIRVFAIPPSFASIFLTKST
KLTCLVTDLTTYDSVTISWTRQNGEAVKTHTNISESHPNATFSAVGEA
SICEDDWNSGERFTCTVTHTDLPSPLKQTISRPKGVALHRPDVYLLPP
AREQLNLRESATITCLVTGFSPADVFVQWMQRGQPLSPEKYVTSAPMP
EPQAPGRYFAHSILTVAEEEWNTGETYTCVVAHEALPNRVTERTVDKS
TGKPTLYNVSLVMSDTAGTCY.

In certain aspects, E402 of SEQ ID NO: 125 can be substituted with any amino acid. In certain aspects, E402 of SEQ ID NO: 125 can be substituted with alanine (A) as follows (alanine substitution indicated by bold underline):

(SEQ ID NO: 129)
GSASAPTLFPLVSCENSPSDTSSVAVGCLAQDFLPDSITFSWKYKNNS
DISSTRGFPSVLRGGKYAATSQVLLPSKDVMQGTDEHVVCKVQHPNGN
KEKNVPLPVIAELPPKVSVFVPPRDGFFGNPRKSKLICQATGFSPRQI
QVSWLREGKQVGSGVTTDQVQAEAKESGPTTYKVTSTLTIKESDWLSQ
SMFTCRVDHRGLTFQQNASSMCVPDQDTAIRVFAIPPSFASIFLTKST
KLTCLVTDLTTYDSVTISWTRQNGEAVKTHTNISESHPNATFSAVGEA
SICEDDWNSGERFTCTVTHTDLPSPLKQTISRPKGVALHRPDVYLLPP
AREQLNLRESATITCLVTGFSPADVFVQWMQRGQPLSPEKYVTSAPMP
EPQAPGRYFAHSILTVSAEEWNTGETYTCVVAHEALPNRVTERTVDKS
TGKPTLYNVSLVMSDTAGTCY.

In certain aspects, E403 of SEQ ID NO: 125 can be substituted with any amino acid. In certain aspects, E403 of SEQ ID NO: 125 can be substituted with alanine (A) as follows (alanine substitution indicated by bold underline):

(SEQ ID NO: 130)
GSASAPTLFPLVSCENSPSDTSSVAVGCLAQDFLPDSITFSWKYKNNS
DISSTRGFPSVLRGGKYAATSQVLLPSKDVMQGTDEHVVCKVQHPNGN
KEKNVPLPVIAELPPKVSVFVPPRDGFFGNPRKSKLICQATGFSPRQI
QVSWLREGKQVGSGVTTDQVQAEAKESGPTTYKVTSTLTIKESDWLSQ
SMFTCRVDHRGLTFQQNASSMCVPDQDTAIRVFAIPPSFASIFLTKST
KLTCLVTDLTTYDSVTISWTRQNGEAVKTHTNISESHPNATFSAVGEA
SICEDDWNSGERFTCTVTHTDLPSPLKQTISRPKGVALHRPDVYLLPP
AREQLNLRESATITCLVTGFSPADVFVQWMQRGQPLSPEKYVTSAPMP
EPQAPGRYFAHSILTVSEAEWNTGETYTCVVAHEALPNRVTERTVDKS
TGKPTLYNVSLVMSDTAGTCY.

In certain aspects, R344 of SEQ ID NO: 125 can be substituted with any amino acid. In certain aspects, R344 of SEQ ID NO: 125 can be substituted with alanine (A) as follows (alanine substitution indicated by bold underline):

(SEQ ID NO: 131)
GSASAPTLFPLVSCENSPSDTSSVAVGCLAQDFLPDSITFSWKYKNNS
DISSTRGFPSVLRGGKYAATSQVLLPSKDVMQGTDEHVVCKVQHPNGN
KEKNVPLPVIAELPPKVSVFVPPRDGFFGNPRKSKLICQATGFSPRQI
QVSWLREGKQVGSGVTTDQVQAEAKESGPTTYKVTSTLTIKESDWLSQ
SMFTCRVDHRGLTFQQNASSMCVPDQDTAIRVFAIPPSFASIFLTKST
KLTCLVTDLTTYDSVTISWTRQNGEAVKTHTNISESHPNATFSAVGEA
SICEDDWNSGERFTCTVTHTDLPSPLKQTISRPKGVALHRPDVYLLPP
AREQLNLAESATITCLVTGFSPADVFVQWMQRGQPLSPEKYVTSAPMP
EPQAPGRYFAHSILTVSEEEWNTGETYTCVVAHEALPNRVTERTVDKS
TGKPTLYNVSLVMSDTAGTCY.

In certain aspects, E345 of SEQ ID NO: 125 can be substituted with any amino acid. In certain aspects, E345 of SEQ ID NO: 125 can be substituted with alanine (A) as follows (alanine substitution indicated by bold underline):

(SEQ ID NO: 132)
GSASAPTLFPLVSCENSPSDTSSVAVGCLAQDFLPDSITFSWKYKNNS
DISSTRGFPSVLRGGKYAATSQVLLPSKDVMQGTDEHVVCKVQHPNGN
KEKNVPLPVIAELPPKVSVFVPPRDGFFGNPRKSKLICQATGFSPRQI
QVSWLREGKQVGSGVTTDQVQAEAKESGPTTYKVTSTLTIKESDWLSQ
SMFTCRVDHRGLTFQQNASSMCVPDQDTAIRVFAIPPSFASIFLTKST
KLTCLVTDLTTYDSVTISWTRQNGEAVKTHTNISESHPNATFSAVGEA
SICEDDWNSGERFTCTVTHTDLPSPLKQTISRPKGVALHRPDVYLLPP
AREQLNLRASATITCLVTGFSPADVFVQWMQRGQPLSPEKYVTSAPMP
EPQAPGRYFAHSILTVSEEEWNTGETYTCVVAHEALPNRVTERTVDKS
TGKPTLYNVSLVMSDTAGTCY.

As indicated, five IgM binding units can form a complex with a J-chain to form a pentameric IgM antibody. The precursor form of the human J-chain includes: MKNHLLFWGVLAVFIKAVHVKAQEDERIVLVDNKCKCARITSRIIRSSEDPNEDIVERNIRIIVPLN NRENISDPTSPLRTRFVYHLSDLCKKCDPTEVELDNQIVTATQSNICDEDSATETCYTYDRNKC YTAVVPLVYGGETKMVETALTPDACYPD (SEQ ID NO: 133). The signal peptide extends from amino acid 1 to amino acid 22 of SEQ ID NO: 133 and the mature human J-chain extends from amino acid 23 to amino acid 159 of SEQ ID NO: 133.

The mature human J-chain includes the amino acid sequence

(SEQ ID NO: 119)
QEDERIVLVDNKCKCARITSRIIRSSEDPNEDIVERNIRIIVPLNNRE
NISDPTSPLRTRFVYHLSDLCKKCDPTEVELDNQIVTATQSNICDEDS
ATETCYTYDRNKCYTAVVPLVYGGETKMVETALTPDACYPD.

The term “J-chain” as used herein refers to the J-chain of native sequence IgM or Igλ antibodies of any animal species. When specified, it can also refer to any functional fragment thereof, derivative thereof, and/or variant thereof, including a mature human J-chain amino acid sequence provided herein as SEQ ID NO: 119. A functional fragment, derivative, and/or variant of a J-chain has at least 90% sequence identity to the reference J-chain and retains the multimerizing function of the reference J-chain.

In certain aspects, the J-chain of the IgM antibody as provided herein includes an amino acid substitution at the amino acid position corresponding to amino acid Y102, T103, N49 or S51 of SEQ ID NO: 119.

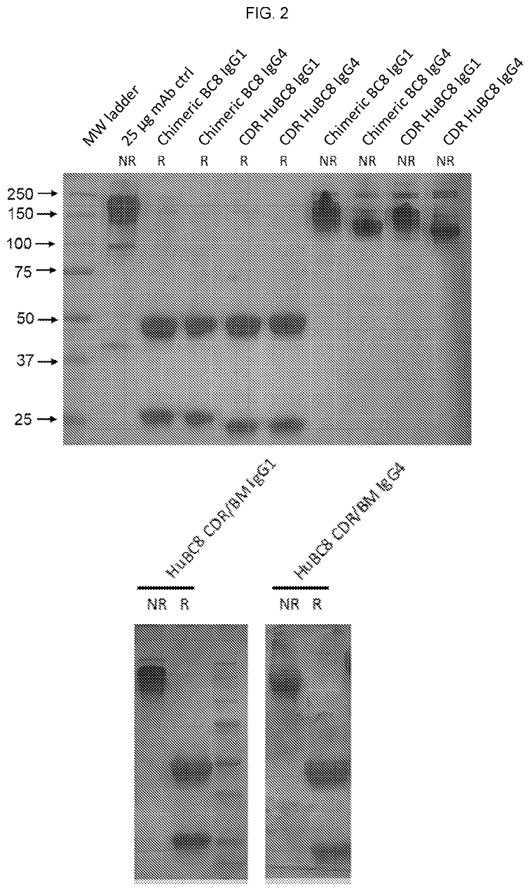

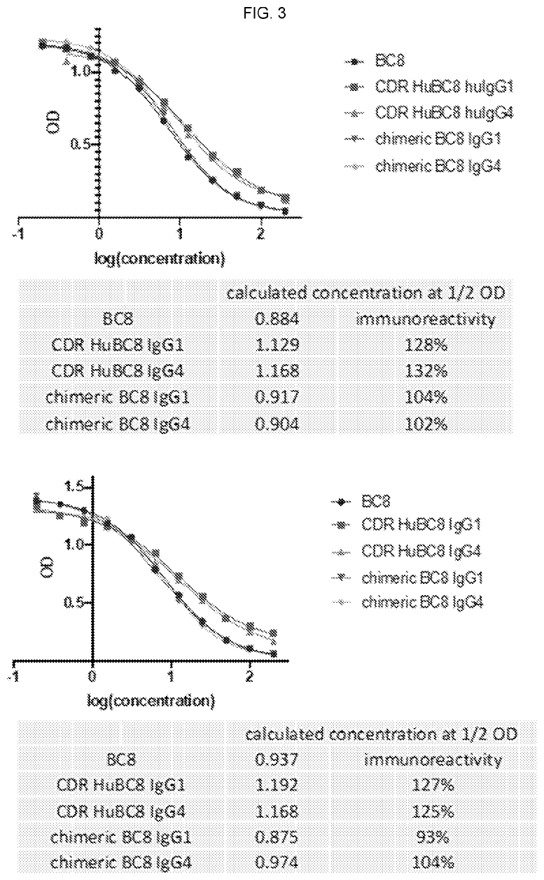

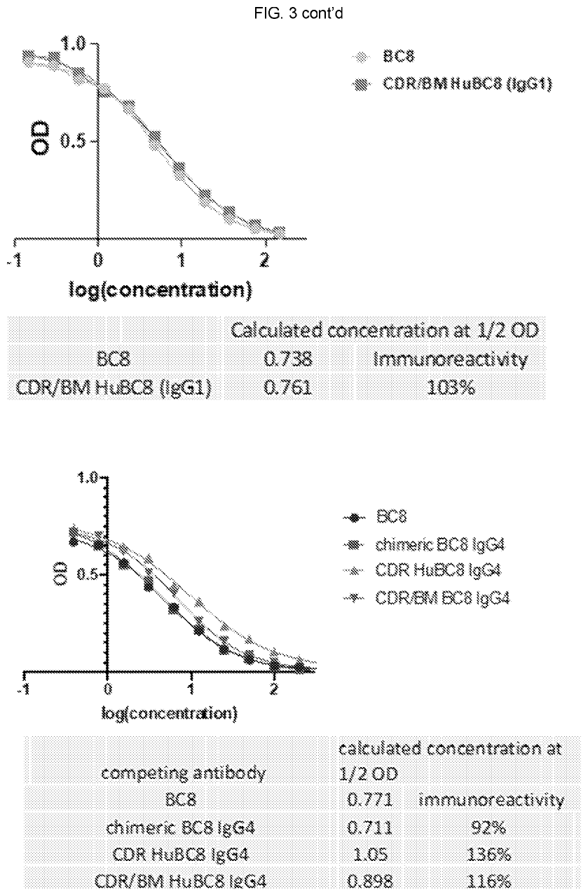

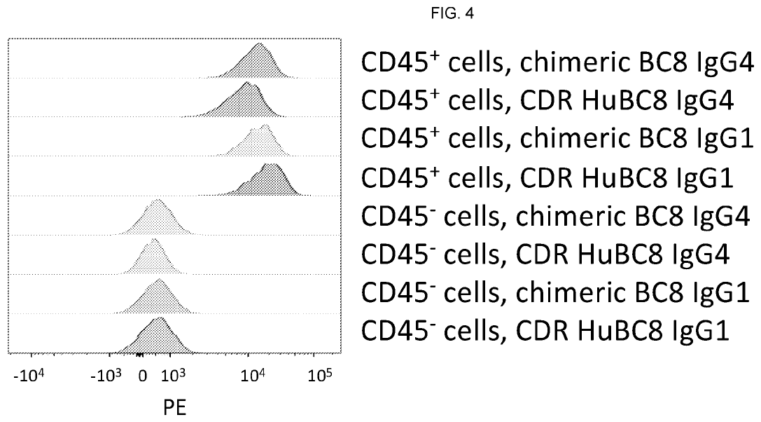

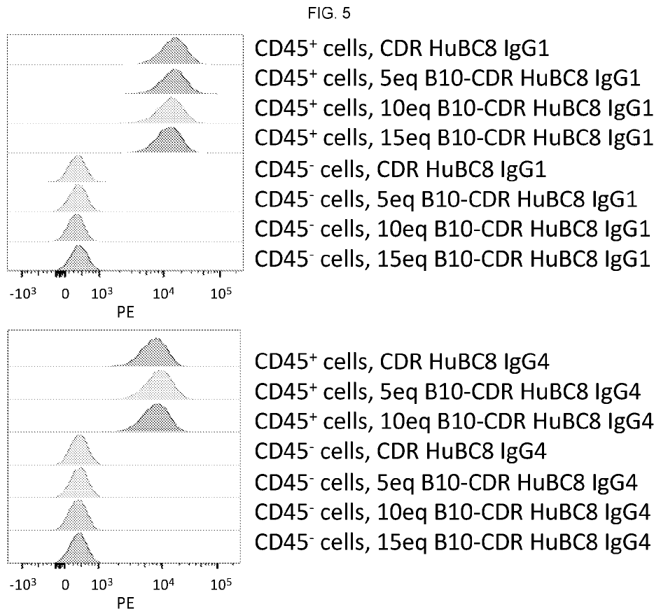

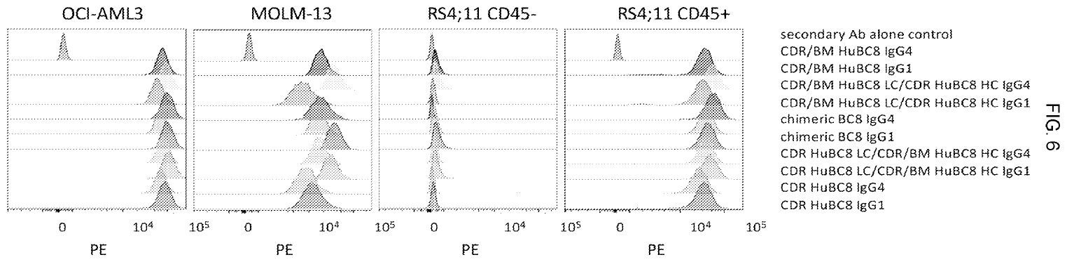

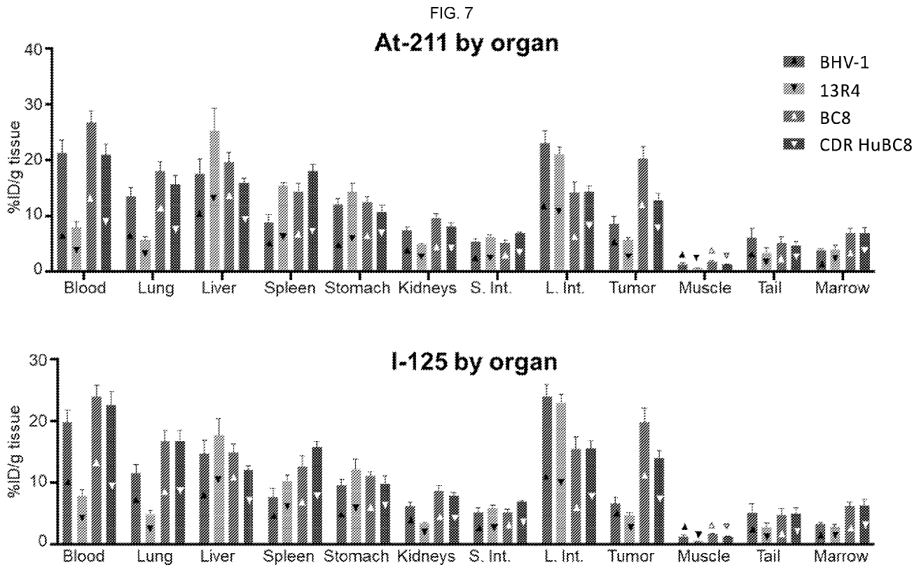

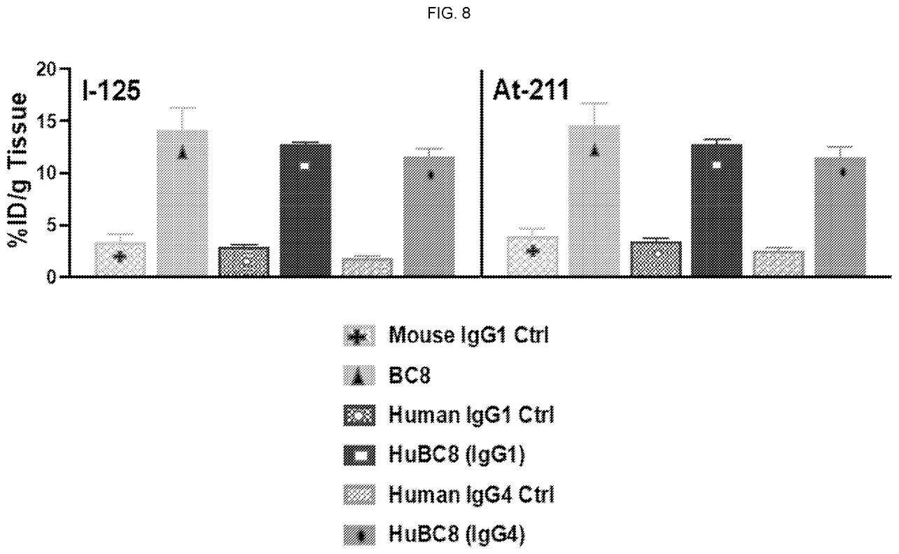

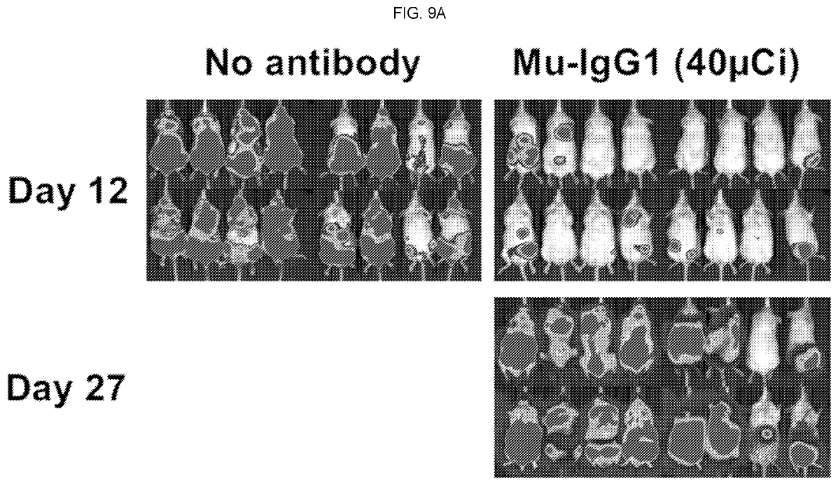

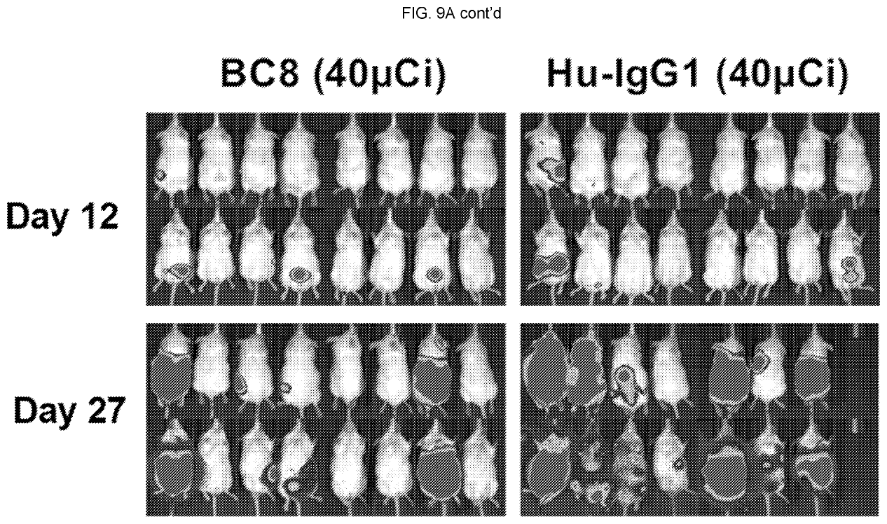

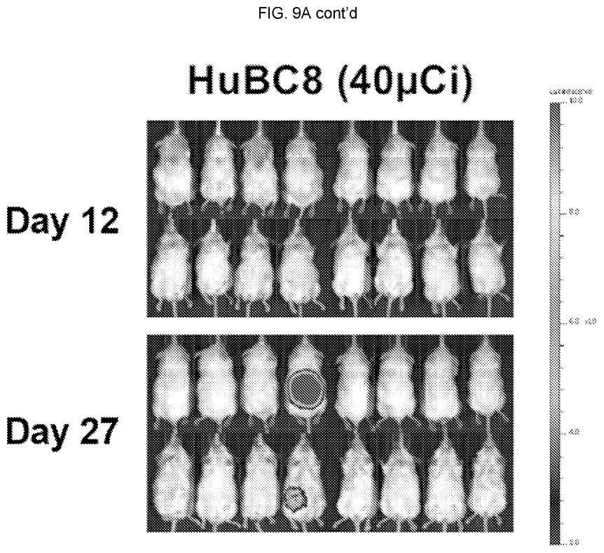

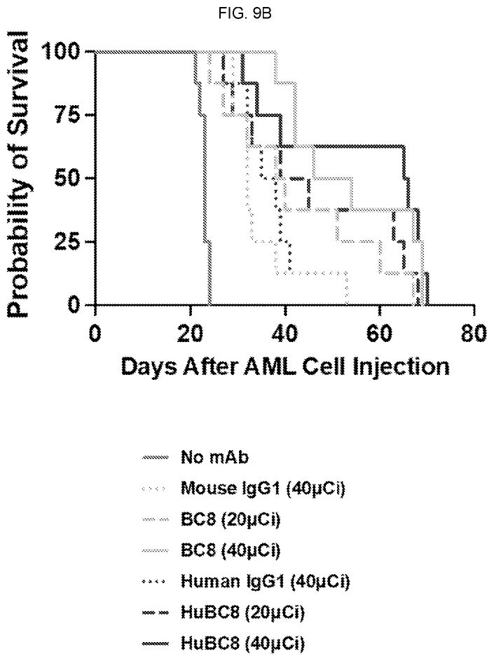

By “an amino acid corresponding to” a position of SEQ ID NO: 119 is meant the amino acid in the sequence of the J-chain of any species which is homologous to the referenced residue in the human J-chain. For example, the position corresponding to Y102 in SEQ ID NO: 119 is conserved in the J-chain amino acid sequences of at least 43 other species. The position corresponding to T103 in SEQ ID NO: 119 is conserved in the J-chain amino acid sequences of at least 37 other species. The positions corresponding to N49 and S51 in SEQ ID NO: 119 are conserved in the J-chain amino acid sequences of at least 43 other species. See FIG. 4 of U.S. Pat. No. 9,951,134 and FIG. 2 of PCT/US2019/020374.

In certain aspects, the amino acid corresponding to Y102 of SEQ ID NO: 119 can be substituted with any amino acid. In certain aspects, the amino acid corresponding to Y102 of SEQ ID NO: 119 can be substituted with alanine (alanine substitution indicated by bold underline):

(SEQ ID NO: 134)
QEDERIVLVDNKCKCARITSRIIRSSEDPNEDIVERNIRIIVPLNNRE
NISDPTSPLRTRFVYHLSDLCKKCDPTEVELDNQIVTATQSNICDEDS
ATETCATYDRNKCYTAVVPLVYGGETKMVETALTPDACYPD,

With serine (serine substitution indicated by bold underline):

(SEQ ID NO: 135)
QEDERIVLVDNKCKCARITSRIIRSSEDPNEDIVERNIRIIVPLNNRE
NISDPTSPLRTRFVYHLSDLCKKCDPTEVELDNQIVTATQSNICDEDS
ATETCSTYDRNKCYTAVVPLVYGGETKMVETALTPDACYPD,

Or with arginine (arginine substitution indicated by bold underline):

(SEQ ID NO: 136)
QEDERIVLVDNKCKCARITSRIIRSSEDPNEDIVERNIRIIVPLNNRE
NISDPTSPLRTRFVYHLSDLCKKCDPTEVELDNQIVTATQSNICDEDS
ATETCRTYDRNKCYTAVVPLVYGGETKMVETALTPDACYPD.

In certain aspects, the amino acid corresponding to T103 of SEQ ID NO: 119 can be substituted with any amino acid. In a particular aspect, the amino acid corresponding to T103 of SEQ ID NO: 119 can be substituted with alanine as follows (alanine substitution indicated by bold underline):

(SEQ ID NO: 137)
QEDERIVLVDNKCKCARITSRIIRSSEDPNEDIVERNIRIIVPLNNRE
NISDPTSPLRTRFVYHLSDLCKKCDPTEVELDNQIVTATQSNICDEDS
ATETCYAYDRNKCYTAVVPLVYGGETKMVETALTPDACYPD.

In certain aspects, the variant J-chain or functional fragment thereof of the IgM antibody as provided herein includes an amino acid substitution at the amino acid position corresponding to amino acid N49 or amino acid S51 of SEQ ID NO: 119, provided that S51 is not substituted with threonine (T), or wherein the J-chain includes amino acid substitutions at the amino acid positions corresponding to both amino acids N49 and S51 of SEQ ID NO: 119.

The amino acids corresponding to N49 and S51 of SEQ ID NO: 119 along with the amino acid corresponding to 150 of SEQ ID NO: 119 include an N-linked glycosylation motif in the J-chain. Accordingly, mutations at N49 and/or S51 (with the exception of a single threonine substitution at S51) can prevent glycosylation at this motif. In certain aspects, the asparagine at the position corresponding to N49 of SEQ ID NO: 119 can be substituted with any amino acid. In certain aspects, the asparagine at the position corresponding to N49 of SEQ ID NO: 119 can be substituted with alanine (A), glycine (G), threonine (T), serine (S) or aspartic acid (D). In a particular aspect the position corresponding to N49 of SEQ ID NO: 119 can be substituted with alanine (A). In a particular aspect the J-chain is a variant human J-chain and includes the amino acid sequence:

(SEQ ID NO: 138)
QEDERIVLVDNKCKCARITSRIIRSSEDPNEDIVERNIRIIVPLNNRE
AISDPTSPLRTRFVYHLSDLCKKCDPTEVELDNQIVTATQSNICDED
SATETCYTYDRNKCYTAVVPLVYGGETKMVETALTPDACYPD.

In certain aspects, the serine at the position corresponding to S51 of SEQ ID NO: 119 can be substituted with any amino acid except threonine. In certain aspects, the serine at the position corresponding to S51 of SEQ ID NO: 119 can be substituted with alanine (A) or glycine (G). In a particular aspect the position corresponding to S51 of SEQ ID NO: 119 can be substituted with alanine (A). In a particular aspect the variant J-chain or functional fragment thereof is a variant human J-chain and includes the amino acid sequence:

(SEQ ID NO: 139)
EDERIVLVDNKCKCARITSRIIRSSEDPNEDIVERNIRIIVPLNNREN
IADPTSPLRTRFVYHLSDLCKKCDPTEVELDNQIVTATQSNICDEDSA
TETCYTYDRNKCYTAVVPLVYGGETKMVETALTPDACYPD.

Particular embodiments include a heterologous polypeptide (e.g., a single-domain antibody binding domain) fused to the J-chain or functional fragment thereof via a peptide linker, e.g., a peptide linker including at least 5 amino acids, but no more than 25 amino acids. In certain aspects, the peptide linker includes (GGGGS)n (SEQ ID NO: 65) wherein n is 1-5.

A single-domain antibody binding domain can be introduced into the J-chain at any location that allows the binding of the binding domain to its binding target without interfering with J-chain function or the function of an associated IgA, IgM, or hybrid IgG antibody. Insertion locations include at or near the C-terminus, at or near the N-terminus or at an internal location that, based on the three-dimensional structure of the J-chain, is accessible. In certain aspects, the antigen-binding domain can be introduced into the mature human J-chain of SEQ ID NO: 119 between cysteine residues 92 and 101 of SEQ ID NO: 119. In a further aspect, the antigen-binding domain can be introduced into the human J-chain of SEQ ID NO: 119 at or near a glycosylation site. In a further aspect, the antigen-binding domain can be introduced into the human J-chain of SEQ ID NO: 119 within 10 amino acid residues from the C-terminus, or within 10 amino acids from the N-terminus.

In particular embodiments, the single-domain antibody is introduced into the native human J-chain sequence of SEQ ID NO: 119 by chemical or chemo-enzymatic derivatization. In particular embodiments, the single-domain antibody is introduced into the native human J-chain sequence of SEQ ID NO: 119 by a chemical linker. In some embodiments, the chemical linker is a cleavable or non-cleavable linker. In particular embodiments, the cleavable linker is a chemically labile linker or an enzyme-labile linker. In some embodiments, the linker is selected from the group including N-succinimidyl-3-(2-pyridyldithio) propionate (SPDP), succinimidyl-4-(N-maleimidomethyl) cyclohexane-I-carboxylate (SMCC), N-succinimidyl-4-(2-pyridylthio) pentanoate (SPP), iminothiolane (IT), afunctional derivatives of imidoesters, active esters, aldehydes, bis-azido compounds, bis-diazonium derivatives, diisocyanates, and bis-active fluorine compounds. In particular embodiments, the modified J-chain is modified by insertion of an enzyme recognition site, and by post-translationally attaching a binding moiety at the enzyme recognition site through a peptide or non-peptide linker.

In certain aspects the modified J-chain can include the formula X[Lₙ]J or J[Lₙ]X, where J includes a mature native J-chain or functional fragment thereof, X includes a heterologous binding domain, and [Lₙ] is a linker sequence including n amino acids, where n is a positive integer from 1 to 100, 1 to 50, or 1 to 25. In certain aspects N is 5, 10, 15, or 20.

J-chains from the following species can also be used in certain embodiments: Pan troglodytes, Pongo abelfi, Callithrix jacchus, Macaca mulatta, Papio Anubis, Saimiri boliviensis, Tupala chinensis, Tursiops truncatus, Orcinus orca, Loxodonta Africana, Leptonychotes weddellil, Ceratotherium simum, Fells catus, Canis familiaris, Alluropoda melanoleuca, Mustela furo, Equus caballus, Cavia porcellus, Camelus ferus, Capra hircus, Chinchilla lanigera, Mesocricetus auratus, Ovis aries, Myotis lucifugus, Pantholops hodgsonii, Bos taurus, Mus musculus, Rattus norvegicus, Echinops telfairi, Oryctolagus cuniculus, Monodelphis domestica, Alligator mississippiensis, Chrysemys picta, Sarcophilus harrisif, Ornithorhynchus anatinus, Melopsittacus undulatus, Anas platyrhynchos, Gallus gallus, Meleagris gallopavo, Falco peregrinus, Zonotrichia albicoilis, and Pteropus alecto.

(V) Expression of Recombinant Antibodies. Chimeric or humanized anti-CD45 antibodies can be produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies.

In particular embodiments, mammalian cells are used as a host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NS0. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. In particular embodiments, expression control sequences are promoters derived from endogenous genes, cytomegalovirus, SV40, adenovirus, and bovine papillomavirus (see Co et al., J. Immunol. 1992, 148:1149).

Once expressed, antibodies can be purified according to standard procedures of the art, including high-performance liquid chromatography (HPLC) purification, column chromatography, gel electrophoresis and the like (see generally, Scopes, Protein Purification (Springer-Verlag, NY, 1982)).

In particular embodiments, antibodies are formed using the Daedalus expression system as described in Pechman et al. (Am J Physiol 294: R1234-R1239, 2008). The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/I of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 39(21) 2011. In some instances, purification by chromatography may not be needed due to the purity of manufacture according to the methods described herein.

(VI) Anti-CD45 Antibody Conjugates. Anti-CD45 antibody conjugates include an anti-CD45 antibody disclosed herein linked to another molecule, other than an additional binding domain. Examples of antibody conjugates include antibody radioisotope conjugates, antibody immunotoxins, antibody-drug conjugates (ADCs), antibody-detectable label conjugates, and antibody-particle conjugates.

Antibody-radioisotope conjugates include an anti-CD45 antibody linked to a radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to anti-CD45 antibodies of the present disclosure include iodine-131 yttrium-90, arsenic-72, arsenic-74, iodine-131, indium-111, and lutetium-177, as well as alpha-emitting radionuclides such as astatine-211, actinium-225, bismuth-212 or bismuth-213. Methods for preparing radioimmunoconjugates are established in the art. Examples of radioimmunoconjugates are commercially available, including Zevalin™ (DEC Pharmaceuticals), and similar methods can be used to prepare radioimmunoconjugates using the antibodies of the disclosure.

Examples of radionuclides that are useful for radiation therapy include ²²⁵Ac and ²²⁷Th. ²²⁵Ac is a radionuclide with the half-life of ten days. As ²²⁵Ac decays the daughter isotopes ²²¹Fr, ²¹³Bi, and ²⁰⁹Pb are formed. ²²⁷Th has a half-life of 19 days and forms the daughter isotope ²²³Ra.

Additional examples of useful radioisotopes include ²²⁸Ac, ¹¹¹Ag, ¹²⁴Am, ⁷⁴As, ²¹¹At, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ¹⁴C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ³H, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, and/or ⁹⁷Zr. Radioisotopes can be used as a type of detectable label called a radiolabel. In particular embodiments, a radioisotope includes ¹³¹I, ⁹⁰Y, and/or ²¹¹At. In particular embodiments, a radioisotope is selected that includes a half-life (t_{1/2}) that enables high-yield radiolabeling and drug delivery. In particular embodiments, a radioisotope is selected that includes a half-life (t_{1/2}) of 7.2 hours. In particular embodiments, a radioisotope is selected that does not emit daughter radionuclides that cause organ toxicity.

In particular embodiments, the anti-CD45 antibody can be formed as an antibody immunotoxin. Antibody immunotoxins include an anti-CD45 antibody disclosed herein conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage.

Antibody-drug conjugates allow for the targeted delivery of a drug moiety to a CD45 expressing cell, in particular embodiments intracellular accumulation therein, where systemic administration of unconjugated drugs may result in unacceptable levels of toxicity to normal cells (Polakis P. (2005) Current Opinion in Pharmacology 5:382-387).

In particular embodiments, antibody-drug conjugates refer to targeted molecules which combine properties of both antibodies and cytotoxic drugs (e.g., chemotherapeutic drugs) by targeting potent cytotoxic drugs to antigen-expressing cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & Iyer (Pharm Res. 32(11): 3470-3479, 2015), which describes considerations for the development of antibody-drug conjugates.

The drug moiety (D) of an antibody-drug conjugate may include any compound, moiety or group that has a cytotoxic or cytostatic effect. Drug moieties may impart their cytotoxic and cytostatic effects by mechanisms including tubulin binding, DNA binding or intercalation, and inhibition of RNA polymerase, protein synthesis, and/or topoisomerase. Exemplary drugs include actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (including monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, and stereoisomers, isosteres, analogs, and derivatives thereof that have cytotoxic activity.

The drug may be obtained from essentially any source; it may be synthetic or a natural product isolated from a selected source, e.g., a plant, bacterial, insect, mammalian or fungal source. The drug may also be a synthetically modified natural product or an analogue of a natural product.

In particular embodiments, the antibody-drug conjugates include an antibody conjugated, i.e., covalently attached, to the drug moiety. In particular embodiments, the anti-CD45 antibody is covalently attached to the drug moiety through a linker. A linker can include any chemical moiety that is capable of linking an antibody, antibody fragment (e.g., antigen binding fragments) or functional equivalent to another moiety, such as a drug moiety. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acid-based linker. The antibody-drug conjugate selectively delivers an effective dose of a drug to cells (e.g., cancer cells) whereby greater selectivity, i.e., a lower efficacious dose, may be achieved while increasing the therapeutic index (“therapeutic window”).

To prepare antibody-drug conjugates, linker-cytotoxin conjugates can be made by conventional methods analogous to those described by Doronina et al. (Bioconjugate Chem. 17: 114-124, 2006). Antibody-drug conjugates with multiple (e.g., four) drugs per antibody can be prepared by partial reduction of the antibody with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA in Dulbecco's phosphate-buffered saline (DPBS). The eluent can be diluted with further DPBS, and the thiol concentration of the antibody can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting ADC mixture can be purified on SEPHADEX G-25 equilibrated in PBS to remove unreacted linker-cytotoxin conjugate, desalted if desired, and purified by size-exclusion chromatography. The resulting ADC can then be sterile filtered, for example, through a 0.2 μm filter, and can be lyophilized if desired for storage. Methods used to produce immunotoxins can similarly be used to prepare antibody-drug conjugates.

Antibody-detectable label conjugates include an anti-CD45 antibody linked to a detectable label. Detectable labels can include any suitable label or detectable group detectable by, for example, optical, spectroscopic, photochemical, biochemical, immunochemical, electrical, optical or chemical means. In particular embodiments, detectable labels can include fluorescent labels, chemiluminescent labels, spectral colorimetric labels, enzymatic labels, and affinity tags.

Fluorescent labels can be particularly useful in cell staining, identification, imaging, and isolation uses. Exemplary fluorescent labels include blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyan1, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen)), CopGFP, AceGFP, avGFP, ZsGreen1, Oregon Green™ (Thermo Fisher Scientific)); Luciferase; orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

Chemiluminescent labels can include lucigenin, luminol, luciferin, isoluminol, theromatic acridinium ester, imidazole, acridinium salt, or oxalate ester.

Spectral colorimetric labels can include colloidal gold or colored glass or plastic (e.g., polystyrene, polypropylene, and latex) beads.

Enzymatic labels can produce, for example, a chemiluminescent signal, a color signal, or a fluorescent signal. Enzymes can include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Affinity tags can include, for example, His tag (HHHHHH (SEQ ID NO: 140)), Flag tag (DYKDDDD (SEQ ID NO: 141), Xpress tag (DLYDDDDK (SEQ ID NO: 142)), Avi tag (GLNDIFEAQKIEWHE (SEQ ID NO: 143)), Calmodulin binding peptide (CBP) tag (KRRWKKNFIAVSAANRFKKISSSGAL (SEQ ID NO: 144)), Polyglutamate tag (EEEEEE (SEQ ID NO: 145)), HA tag (YPYDVPDYA (SEQ ID NO: 146)), Myc tag (EQKLISEEDL (SEQ ID NO: 147)), Strep tag (WRHPQFGG (SEQ ID NO: 148)), STREP® tag II (WSHPQFEK (SEQ ID NO: 149); IBA Institut fur Bioanalytik, Germany; see, e.g., U.S. Pat. No. 7,981,632), Softag 1 (SLAELLNAGLGGS (SEQ ID NO: 150)), Softag 3 (TQDPSRVG (SEQ ID NO: 151)), and V5 tag (GKPIPNPLLGLDST (SEQ ID NO: 152)).

Antibody-particle conjugates include an antibody linked to a particle. In particular embodiments, particles include microparticles, nanoparticles, nanoshells, nanobeads, microbeads, or nanodots. Particles can include, for example, latex beads, polystyrene beads, fluorescent beads, and/or colored beads, and can be made from organic matter and/or inorganic matter. They can be made of any suitable materials that allow for the conjugation of capture proteins, such as anti-CD45 antibodies disclosed herein, to their surface. Examples of suitable materials include: ceramics, glass, polymers, and magnetic materials. Suitable polymers include polystyrene, poly-(methyl methacrylate), poly-(lactic acid), (poly-(lactic-co-glycolic acid)), polyesters, polyethers, polyolefins, polyalkylene oxides, polyamides, polyurethanes, polysaccharides, celluloses, polyisoprenes, methylstyrene, acrylic polymers, thoria sol, latex, nylon, Teflon cross-linked dextrans (e.g., Sepharose), chitosan, agarose, and cross-linked micelles. Additional examples include carbon graphited, titanium dioxide, and paramagnetic materials. See, e.g., “Microsphere Detection Guide” from Bangs Laboratories, Fishers Ind. In particular embodiments, microparticles can be made of one or more materials. In particular embodiments, microparticles are paramagnetic microparticles. Particular embodiments utilize carboxy-modified polystyrene latex (CML) flow cytometry beads and/or magnetic MagPlex® (Luminex, Austin, TX) flow cytometry beads. In particular embodiments, particles can carry a payload.

In particular embodiments, an antibody as disclosed herein can be linked to a conjugate by any method known in the art. In particular embodiments, the constant region can be modified to allow for site specific conjugation. Such techniques include the use of naturally occurring or engineered cysteine residues, disulfide bridges, poly-histidine sequences, glycoengineering tags, and transglutaminase recognition sequences. Antibody fragments can also be modified for site-specific conjugation, see for example, Kim et al., Mol Cancer Ther 2008; 7(8).

(VII) Chimeric Antigen Receptors (CARs) & Engineered T Cell Receptors (eTCR). Anti-CD45 antibodies disclosed herein can be utilized within chimeric antigen receptors (CAR) and/or engineered T cell receptors (eTCR).

CAR include several distinct subcomponents that allow genetically modified cells (e.g., regulatory T cells) to recognize and kill cells expressing an antigen (e.g., CD45). The subcomponents include at least an extracellular component and an intracellular component. The extracellular component includes a binding domain that specifically binds a CD45 epitope that is preferentially present on the surface of cells or in the area thereof. When the binding domain binds such epitopes, the intracellular component activates the cell to destroy the bound cell. CAR additionally include a transmembrane domain that directly or indirectly links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted epitope.

eTCR disclosed herein include an anti-CD45 antibody disclosed herein linked to the C_{α} and/or C_{β} chains of a TCR. A TCR is a heterodimeric fusion protein that typically includes an α and β chain. Each chain includes a variable region (V_{α} and V_{β}) and a constant region (C_{α} and C_{β}). In particular embodiments, an eTCR does not include the native TCR variable region but does include the native TCR constant region. In particular embodiments, the eTCR includes an anti-CD45 antibody as the variable region of the α and β chain. In particular embodiments, eTCR include a C_{α} and/or C_{β} chain sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR C_{α} or C_{β}.

Particular embodiments of binding domains include an anti-CD45 antibody and/or the CDRs thereof as disclosed herein, such as those provided in SEQ ID NOs: 37-64.

CAR and eTCR can additionally include spacer regions, transmembrane domains, intracellular effector domains, transduction markers, and tags.

Spacer regions are used to create appropriate distances and/or flexibility between subcomponents of a protein. Spacer regions typically include 10 to 250 amino acids, 10 to 200 amino acids, 10 to 150 amino acids, 10 to 100 amino acids, 10 to 50 amino acids, or 10 to 25 amino acids. Exemplary spacer regions include all or a portion of an immunoglobulin hinge region.

Transmembrane domains typically have a three-dimensional structure that is thermodynamically stable in a cell membrane, and generally ranges in length from 15 to 30 amino acids. The structure of a transmembrane domain can include an a helix, a β barrel, a β sheet, a β helix, or any combination thereof. Transmembrane domains can include at least the transmembrane region(s) of the α, β or ζ chain of a T-cell receptor, CD28, CD27, CD3, CD45, CD4, CD5, CD8, CD9, CD16, CD22; CD45, CD37, CD64, CD80, CD86, CD134, CD137 and CD154.

A transmembrane domain can include one or more additional amino acids adjacent to the transmembrane region, e.g., one or more amino acid within the extracellular region of the expressed protein (e.g., up to 15 amino acids of the extracellular region) and/or one or more additional amino acids within the intracellular region of the expressed protein (e.g., up to 15 amino acids of the intracellular components).

Intracellular effector domains activate the expressing cell when the binding domain binds antigen (CD45). The term “effector domain” is thus meant to include any portion of the intracellular domain sufficient to transduce an activation signal.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), LIGHT, NKG2C, B7-H3, a ligand that specifically binds with CD83, CDS, ICAM-1, GITR, BAFFR, SLAMF7, NKp80 (KLRF1), CD127, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD137), OX40, PD-1, ICOS, lymphocyte function-associated antigen-1 (LFA-1), NKG2C, and a ligand that specifically binds with CD83.

Transduction markers may be selected from, for example, at least one of a truncated CD19 (tCD19; see Budde et al., Blood 122: 1660, 2013); a truncated human EGFR (tEGFR; see Wang et al., Blood 118: 1255, 2011); an extracellular domain of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278). Methods to genetically modify cells to express CAR are well-known in the art.

CAR and eTCR can additionally include tags, such as the tags described as affinity tags elsewhere herein.

(VIII) Compositions and Formulations. Any of the antibodies described herein (e.g., anti-CD45 antibodies, multi-domain binding molecules, antibody conjugates) in any exemplary format can be formulated alone or in combination into compositions for administration to subjects. Additionally, nucleic acids encoding the antibodies can also be formulated into compositions for administration (e.g., nucleic acids encapsulated within nanoparticles (e.g., liposomes or polymer-based nanoparticles) and/or as part of a vector delivery system (e.g., a viral vector or plasmid). Antibodies (e.g., anti-CD45 antibodies, multi-domain binding molecules, antibody conjugates) and/or nucleic acids encoding antibodies are collectively referred to herein as “active ingredients”.

Salts and/or pro-drugs of the active ingredients can also be used.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of nanoparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

In certain examples, cells are genetically modified to express a protein including a disclosed binding domain (as part of, for example, a CAR or eTCR). In these embodiments, genetically modified cells can be prepared as formulations for delivery in buffers such as Hanks' solution, Ringer's solution, or physiological saline.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In particular embodiments, cells are in a formulation volume of a liter or less, 500 ml or less, 250 ml or less, or 100 ml or less. Hence, the density of administered cells is typically greater than 10⁴ cells/ml, 10⁵ cells/ml, 10⁶ cells/ml, 10⁷ cells/ml, or 10⁸ cells/ml.

Any composition or formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions and formulations can be prepared to meet sterility, pyrogenicity, general safety, and purity standards as required by U.S. FDA Office of Biological Standards and/or other relevant foreign regulatory agencies.

(IX) Methods of Use. Methods disclosed herein include treating subjects. Subjects include, e.g., humans, veterinary animals (dogs, cats, reptiles, birds) livestock (e.g., horses, cattle, goats, pigs, chickens) and research animals (e.g., monkeys, rats, mice, fish). Treating subjects includes delivering therapeutically effective amounts. Therapeutically effective amounts include those that provide effective amounts, prophylactic treatments and/or therapeutic treatments.

An “effective amount” is the amount of a composition or formulation necessary to result in a desired physiological change in the subject. Effective amounts are often administered for research purposes. Effective amounts disclosed herein can cause a statistically-significant effect in an animal model or in vitro assay relevant to the assessment of a condition's development, progression, and/or resolution. In particular embodiments, a condition is a CD45-related condition.

A “prophylactic treatment” includes a treatment administered to a subject who does not display signs or symptoms of a condition or displays only early signs or symptoms of a condition such that treatment is administered for the purpose of diminishing or decreasing the risk of developing the condition further. Thus, a prophylactic treatment functions as a preventative treatment against a condition. In particular embodiments, prophylactic treatments reduce, delay, or prevent the worsening of a condition.

A “therapeutic treatment” includes a treatment administered to a subject who displays symptoms or signs of a condition and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment, or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, an increase in life expectancy, prolonged subject life, induced chemo- or radiosensitivity in cancer cells, inhibited cancer cell proliferation, reduced cancer-associated pain, and/or reduced relapse or re-occurrence of cancer following treatment.

In particular embodiments, therapeutically effective amounts induce an immune response. The immune response can be against a cancer cell, such as a CD45-expressing cancer cell.

Exemplary CD45-related conditions include any condition in which CD45 is expressed on the surface of the cell (CD45-positive cell). In particular embodiments, a CD45-related condition or conditions that can be targeted by targeting CD45 includes a hematologic malignancy. In particular embodiments, a hematologic malignancy includes leukemia, myeloma, or lymphoma. In particular embodiments, leukemia includes acute myelogenous leukemia (AML), acute lymphocytic leukemia (ALL), myelodysplastic syndromes (MDS), chronic lymphocytic leukemia (CLL), or chronic myelogenous leukemia (CML), acute promyelocytic leukemia, acute mixed lineage leukemia, hairy cell leukemia, and large granular lymphocytic leukemia. In particular embodiments, myeloma includes multiple myeloma. In particular embodiments, lymphomas include Hodgkin's lymphoma, non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma, T-cell lymphoma, and other B-cell malignancies. In particular embodiments, a CD45-related condition includes AML. In particular embodiments, AML includes relapsed AML or refractory AML.

In particular embodiments, anti-CD45 conjugates can be used as a conditioning treatment in patients before they undergo allogeneic or autologous hematopoietic cell transplant (HCT). In particular embodiments, an anti-CD45 conjugated to a radioisotope selected from ¹³¹I, ⁹⁰Y, and ²¹¹At can be used as a conditioning treatment in patients before they undergo allogeneic or autologous HCT. In particular embodiments, an anti-CD45 conjugated to drug or immunotoxin can be used as a conditioning treatment in patients before they undergo allogeneic or autologous HCT. In particular embodiments, anti-CD45 conjugates improve the engraftment of gene edited stem cell products and tolerability of HCT. In particular embodiments, treatment with anti-CD45 conjugates result in minimal residual disease burden.

In particular embodiments, patients that are “in need of” a hematopoietic stem cell transplant include patients that exhibit a defect or deficiency in one or more blood cell types, as well as patients having a stem cell disorder, autoimmune disease, cancer, or other pathology described herein, Hematopoietic stem cells generally exhibit 1) multi-potency, and can thus differentiate into multiple different blood lineages including granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g. monocytes, macrophages), dendritic cells, miicroglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to reconstitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type I diabetes, or another autoimmune pathology described herein. In some embodiments, the subject is. In need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolpidoses, metachromatic leukodystrophy, or any other diseases or disorders which may benefit from the treatments and therapies disclosed herein and including severe combined immunodeficiency, Wiscott-Aldrich syndrome, hyper immunoglobin M (IgM) syndrome. Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000). Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

In particular embodiments, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoetic stem or progenitor cell population from a subject's bone marrow tissue) and/or by engrafting or transplanting stem cells in a subjects target tissues. For example, Type I diabetes has been shown to be cured by hematopoietic stem cell transplant and may benefit from conditioning in accordance with the compositions and methods described herein, Additional disorders that can be treated using the compositions and methods described herein include sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease. Hurlers Disease, sphingolipidoses, metachromatic leukodystrophy, or any other diseases or disorders which may benefit from the treatments and therapies disclosed herein and including severe combined immunodeficiency, Wiscott-Aldrich syndrome, hyper immunoglobin M (IgM) syndrome. Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of condition, stage of condition, previous or concurrent therapeutic interventions, idiopathy of the subject and route of administration.

Useful doses can range from 0.1 to 5 μg/kg or from 0.5 to 1 pg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Useful doses can range from 0.1 to 5 μCi/kg or from 0.5 to 1 μCi/kg. In other examples, a dose can include 1 μCi/kg, 15 μCi/kg, 30 μCi/kg, 50 μCi/kg, 55 μCi/kg, 70 μCi/kg, 90 μCi/kg, 150 μCi/kg, 350 μCi/kg, 500 μCi/kg, 750 μCi/kg, or 1000 μCi/kg. In particular embodiments, a dose includes up to 500 μCi/kg.

Exemplary doses of cell-based compositions can include 104 to 10⁹ cells/kg body weight, or 103 to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

The compositions described herein can be administered by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, oral, subcutaneous, and/or sublingual administration. Formulations are generally be administered by injection.

(X) Kits. Also provided herein are kits including at least one antibody or sequences encoding at least one antibody disclosed herein. Kits may be formed with components to practice, for example, the methods described herein. In particular embodiments, the kit includes a humanized anti-CD45 antibody, a multidomain binding molecule, an antibody conjugate, or a multimerized antibody as described herein, or sequences encoding a humanized anti-CD45 antibody, a multidomain binding molecule, an antibody conjugate, or a multimerized antibody as described herein. In particular embodiments, the kit includes cells expressing CAR or eTCR or compositions to modify cells to express CAR or eTCR. The kit may include material(s), which may be desirable from a user standpoint, such as a buffer(s), a diluent(s), a standard(s), and/or other material useful in sample processing, washing, or conducting any other step of the method described herein.

In particular embodiments, a kit includes an antibody conjugate or sequence encoding an antibody conjugate and any other materials needed for treatment of CD45-related conditions.

The kit according to the present disclosure may also include instructions for carrying out the method. Instructions included in the kit of the present disclosure may be affixed to packaging material or may be included as a package insert. While instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site which provides instructions.

Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (XI) Exemplary Embodiments

1. An antibody or fragment thereof that binds CD45 including a light chain variable region includes a complementarity determining region (CDR) light (L)1, CDRL2, and CDRL3 and a heavy chain variable region includes a CDR heavy (H)1, CDRH2, and CDRH3;
   wherein the CDRL1 includes the sequence as set forth in SEQ ID NO: 37, the CDRL2 includes the sequence as set forth in SEQ ID NO: 38, the CDRL3 includes the sequence as set forth in SEQ ID NO: 39, the CDRH1 includes the sequence as set forth in SEQ ID NO: 40, the CDRH2 includes the sequence as set forth in SEQ ID NO: 41, and the CDRH3 includes the sequence as set forth in SEQ ID NO: 42 according to North;
   wherein the CDRL1 includes the sequence as set forth in SEQ ID NO: 43, the CDRL2 includes the sequence including LAS, the CDRL3 includes the sequence as set forth in SEQ ID NO: 39, the CDRH1 includes the sequence as set forth in SEQ ID NO: 44, the CDRH2 includes the sequence as set forth in SEQ ID NO: 45 and the CDRH3 includes the sequence as set forth in SEQ ID NO: 42 according to IMGT;
   wherein the CDRL1 includes the sequence as set forth in SEQ ID NO: 37, the CDRL2 includes the sequence as set forth in SEQ ID NO: 46, the CDRL3 includes the sequence as set forth in SEQ ID NO: 39, the CDRH1 includes the sequence as set forth in SEQ ID NO: 47, the CDRH2 includes the sequence as set forth in SEQ ID NO: 48, and the CDRH3 includes the sequence as set forth in SEQ ID NO: 49 according to Kabat;
   wherein the CDRL1 includes the sequence as set forth in SEQ ID NO: 37, the CDRL2 includes the sequence as set forth in SEQ ID NO: 46, the CDRL3 includes the sequence as set forth in SEQ ID NO: 39, the CDRH1 includes the sequence as set forth in SEQ ID NO: 50, the CDRH2 includes the sequence as set forth in SEQ ID NO: 51, and the CDRH3 includes the sequence as set forth in SEQ ID NO: 49 according to Chothia;
   wherein the CDRL1 includes the sequence as set forth in SEQ ID NO: 52, the CDRL2 includes the sequence as set forth in SEQ ID NO: 53, the CDRL3 includes the sequence as set forth in SEQ ID NO: 54, the CDRH1 includes the sequence as set forth in SEQ ID NO: 55, the CDRH2 includes the sequence as set forth in SEQ ID NO: 56, and the CDRH3 includes the sequence as set forth in SEQ ID NO: 57 according to Contact; or
   wherein CDRL1 includes the sequence as set forth in SEQ ID NO: 52, CDRL2 includes the sequence as set forth in SEQ ID NO: 53, CDRL3 includes the sequence as set forth in SEQ ID NO: 54, CDRH1 includes the sequence as set forth in SEQ ID NO: 55, CDRH2 includes the sequence as set forth in SEQ ID NO: 58, and CDRH3 includes the sequence as set forth in SEQ ID NO: 57 according to Contact;
   wherein the antibody or fragment thereof further includes human variable framework regions and/or a human constant region.
2. The antibody or fragment thereof of embodiment 1, wherein the light chain variable region includes a murine BC8 light chain variable region, a humanized anti-CD45 (CDR HuBC8) light chain variable region, or a humanized anti-CD45 with back mutations (CDR/BM HuBC8) light chain variable region.
3. The antibody or fragment thereof of embodiment 2, wherein the murine BC8 light chain variable region includes the sequence as set forth in SEQ ID NO: 59 or a sequence having at least 90%, 95%, or 98% sequence identity to SEQ ID NO: 59, wherein the antibody or fragment thereof retains CD45 binding.
4. The antibody or fragment thereof of embodiment 2, wherein the CDR HuBC8 light chain variable region includes the sequence as set forth in SEQ ID NO: 61 or a sequence having at least 90%, 95%, or 98% sequence identity to SEQ ID NO: 61, wherein the antibody or fragment thereof retains CD45 binding.
5. The antibody or fragment thereof of embodiment 2, wherein the CDR/BM HuBC8 light chain variable region includes the sequence as set forth in SEQ ID NO: 63 or a sequence having at least 90%, 95%, or 98% sequence identity to SEQ ID NO: 63, wherein the antibody or fragment thereof retains CD45 binding.
6. The antibody or fragment thereof of any of embodiments 1-5, wherein the heavy chain variable region includes a murine BC8 heavy chain variable region, a CDR HuBC8 heavy chain variable region, or a CDR/BM HuBC8 heavy chain variable region.
7. The antibody or fragment thereof of embodiment 6, wherein the murine BC8 heavy chain variable region includes the sequence as set forth in SEQ ID NO: 60 or a sequence having at least 90%, 95%, or 98% sequence identity to SEQ ID NO: 60, wherein the antibody or fragment thereof retains CD45 binding.
8. The antibody or fragment thereof of embodiment 6, wherein the CDR HuBC8 chain variable region includes the sequence as set forth in SEQ ID NO: 62 or a sequence having at least 90%, 95%, or 98% sequence identity to SEQ ID NO: 62, wherein the antibody or fragment thereof retains CD45 binding.
9. The antibody or fragment thereof of embodiment 6, wherein the CDR/BM HuBC8 heavy chain variable region includes the sequence as set forth in SEQ ID NO: 64 or a sequence having at least 90%, 95%, or 98% sequence identity to SEQ ID NO: 64, wherein the antibody or fragment thereof retains CD45 binding.
10. The antibody or fragment thereof of any of embodiments 1-9, wherein the antibody or fragment thereof further includes the human constant region.
11. The antibody or fragment thereof of embodiment 10, wherein the human constant region includes a human light chain constant region and/or a human heavy chain constant region.
12. The antibody or fragment thereof of embodiment 11, wherein the human light chain constant region includes a human Igκ light chain constant region or a human IgA light chain constant region.
13. The antibody or fragment thereof of embodiments 11 or 12, wherein the human light chain constant region includes a human Igκ light chain constant region.
14. The antibody or fragment thereof of embodiments 12 or 13, wherein the human Igκ light chain constant region includes the sequence as set forth in SEQ ID NO: 26.
15. The antibody or fragment thereof of any of embodiments 11-14, wherein the human heavy chain constant region includes a human IgG1 heavy chain constant region, a human IgG4_S228P heavy chain constant region, a human IgG4 heavy chain constant region, a human IgG2 heavy chain constant region, or a human IgG3 heavy chain constant region.
16. The antibody or fragment thereof of any of embodiments 11-14, wherein the human heavy chain constant region includes a human IgG1 heavy chain constant region or a human IgG4_S228P heavy chain constant region.
17. The antibody or fragment thereof of embodiments 15 or 16, wherein the human IgG1 heavy chain constant region includes the sequence as set forth in SEQ ID NO: 28.
18. The antibody or fragment thereof of embodiments 15 or 16, wherein the human IgG4_S228P heavy chain constant region includes the sequence as set forth in SEQ ID NO: 36.
19. The antibody or fragment thereof of any of embodiments 1-18, wherein the light chain variable region further includes a signal peptide.
20. The antibody or fragment thereof of any of embodiments 1-19, wherein the heavy chain variable region further includes a signal peptide.
21. The antibody or fragment thereof of embodiments 19 or 20, wherein the signal peptide includes a sequence as set forth in SEQ ID NO: 1, SEQ ID NO: 2, or SEQ ID NO: 3.
22. The antibody or fragment thereof of any of embodiments 1-21, wherein the light chain variable region includes a murine BC8 light chain variable region and wherein the antibody or fragment thereof includes a human Igκ light chain constant region.
23. The antibody or fragment thereof of embodiment 22, including the sequence as set forth in SEQ ID NO: 4 or a sequence having at least 90%, 95%, or 98% sequence identity to SEQ ID NO: 4, wherein the antibody or fragment thereof retains CD45 binding.
24. The antibody or fragment thereof of any of embodiments 1-23, wherein the heavy chain variable region includes a murine BC8 heavy chain variable region and wherein the antibody or fragment thereof includes a human IgG1 heavy chain constant region.
25. The antibody or fragment thereof of embodiment 24, including the sequence as set forth in SEQ ID NO: 5 or a sequence having at least 90%, 95%, or 98% sequence identity to SEQ ID NO: 5, wherein the antibody or fragment thereof retains CD45 binding.
26. The antibody or fragment thereof of any of embodiments 1-25, wherein the heavy chain variable region includes a murine BC8 heavy chain variable region and wherein the antibody or fragment thereof includes a human IgG4_S228P heavy chain constant region.
27. The antibody or fragment thereof of embodiment 26, including the sequence as set forth in SEQ ID NO: 6 or a sequence having at least 90%, 95%, or 98% sequence identity to SEQ ID NO: 6, wherein the antibody or fragment thereof retains CD45 binding.
28. The antibody or fragment thereof of any of embodiments 1-27, wherein the light chain variable region includes a CDR HuBC8 light chain variable region and wherein the antibody or fragment thereof includes a human Igκ light chain constant region.
29. The antibody or fragment thereof of embodiment 28, including the sequence as set forth in SEQ ID NO: 7 or a sequence having at least 90%, 95%, or 98% sequence identity to SEQ ID NO: 7, wherein the antibody or fragment thereof retains CD45 binding.
30. The antibody or fragment thereof of any of embodiments 1-29, wherein the heavy chain variable region includes a CDR HuBC8 heavy chain variable region and wherein the antibody or fragment thereof includes a human IgG1 heavy chain constant region.
31. The antibody or fragment thereof of embodiment 30, including the sequence as set forth in SEQ ID NO: 8 or a sequence having at least 90%, 95%, or 98% sequence identity to SEQ ID NO: 8, wherein the antibody or fragment thereof retains CD45 binding.
32. The antibody or fragment thereof of any of embodiments 1-31, wherein the heavy chain variable region includes a CDR HuBC8 heavy chain variable region and wherein the antibody or fragment thereof includes a human IgG4_S228P heavy chain constant region.
33. The antibody or fragment thereof of embodiment 32, including the sequence as set forth in SEQ ID NO: 9 or a sequence having at least 90%, 95%, or 98% sequence identity to SEQ ID NO: 9, wherein the antibody or fragment thereof retains CD45 binding.
34. The antibody or fragment thereof of any of embodiments 1-33, wherein the light chain variable region includes a CDR/BM HuBC8 light chain variable region and wherein the antibody or fragment thereof includes a human Igκ light chain constant region.
35. The antibody or fragment thereof of embodiment 34, including the sequence as set forth in SEQ ID NO: 10 or a sequence having at least 90%, 95%, or 98% sequence identity to SEQ ID NO: 10, wherein the antibody or fragment thereof retains CD45 binding.
36. The antibody or fragment thereof of any of embodiments 1-35, wherein the heavy chain variable region includes a CDR/BM HuBC8 heavy chain variable region and wherein the antibody or fragment thereof includes a human IgG1 heavy chain constant region.
37. The antibody or fragment thereof of embodiment 36, including the sequence as set forth in SEQ ID NO: 11 or a sequence having at least 90%, 95%, or 98% sequence identity to SEQ ID NO: 11, wherein the antibody or fragment thereof retains CD45 binding.
38. The antibody or fragment thereof of any of embodiments 1-37, wherein the heavy chain variable region includes a CDR/BM HuBC8 heavy chain variable region and wherein the antibody or fragment thereof includes a human IgG4_S228P heavy chain constant region.
39. The antibody or fragment thereof of embodiment 38, including the sequence as set forth in SEQ ID NO: 12 or a sequence having at least 90%, 95%, or 98% sequence identity to SEQ ID NO: 12, wherein the antibody or fragment thereof retains CD45 binding.
40. A multi-domain binding molecule including at least two binding domains wherein at least one binding domain includes the antibody or fragment thereof of any of embodiments 1-39.
41. The multi-domain binding molecule of embodiment 40, wherein the multi-domain binding molecule includes an immune cell engaging molecule.
42. The multi-domain binding molecule of embodiment 41, wherein the immune cell engaging molecule activates a B cell, T cell, natural killer (NK) cell, or macrophage.
43. The multi-domain binding molecule of embodiment 42, wherein the T cell is a CD3 T cell, a CD4 T cell, a CD8 T cell, a central memory T cell, an effector memory T cell, and/or a naïve T cell.
44. The multi-domain binding molecule of any of embodiments 41-43, wherein a binding domain of the immune cell engaging molecule binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Ra, or MARCO.
45. The multi-domain binding molecule of any of embodiments 40-44, wherein the at least two binding domains include at least two copies of the antibody or fragment thereof of embodiment 1.
46. The multi-domain binding molecule of embodiment 45, wherein the at least two copies are joined by a protein linker.
47. The multi-domain binding molecule of embodiment 46, wherein the protein linker is a Gly-Ser linker.
48. The multi-domain binding molecule of embodiment 47, wherein the Gly-Ser linker is (GlyₓSer_{y})ₙ wherein x and y are independently an integer from 0 to 10 provided that x and y are not both 0 and wherein n is an integer of 1, 2, 3, 4, 5, 6, 7, 8, 9 or 10.
49. The multi-domain binding molecule of any of embodiments 40-48, including 2, 3, 4, 5, 6, 7, 8, 9, or 10 copies of the antibody or fragment thereof of any of embodiments 1-39.
50. The multi-domain binding molecule of any of embodiments 40-49, wherein the multi-domain binding molecule is a dimer, trimer, tetramer, pentamer, hexamer, or heptamer.
51. The multi-domain binding molecule of any of embodiments 46-50, wherein the at least two copies are linked to an Fc region of an antibody.
52. The multi-domain binding molecule of embodiment 51, wherein the Fc region is an IgA Fc region or an IgM Fc region.
53. The multi-domain binding molecule of embodiments 51 or 52, wherein the Fc region is an Igλ Fc region having the sequence as set forth in SEQ ID NOs: 117 and 118.
54. The multi-domain binding molecule of embodiments 51 or 52, wherein the Fc region is an IgM Fc region having the sequence as set forth in SEQ ID NOs: 122-132.
55. The multi-domain binding molecule of any of embodiments 51-54, wherein the Fc region includes a multimerizing fragment of the IgA Fc region or a multimerizing fragment of the IgM Fc region.
56. The multi-domain binding molecule of embodiment 55, wherein the multimerizing fragment of the IgA Fc region includes the IgA tailpiece.
57. The multi-domain binding molecule of embodiment 56, wherein the IgA tailpiece has the sequence of residues 331-352 as set forth in SEQ ID NO: 117 or the sequence of residues 318-340 as set forth in SEQ ID NO: 118.
58. The multi-domain binding molecule of embodiment 55, wherein the multimerizing fragment of the IgA Fc region includes the IgA CA3 domain and the IgA tailpiece.
59. The multi-domain binding molecule of embodiment 55, wherein the multimerizing fragment of the IgA Fc region includes the IgA CA2 domain, the IgA CA3 domain, and the IgA tailpiece.
60. The multi-domain binding molecule of embodiment 55, wherein the multimerizing fragment of the IgA Fc region includes the IgA CA1 domain, the IgA CA2 domain, the IgA CA3 domain, and the IgA tailpiece.
61. The multi-domain binding molecule of embodiment 55, wherein the multimerizing fragment of the IgM Fc region includes the IgM tailpiece.
62. The multi-domain binding molecule of embodiment 55, wherein the multimerizing fragment of the IgM Fc region includes the Cμ4 domain and the IgM tailpiece.
63. The multi-domain binding molecule of embodiment 55, wherein the multimerizing fragment of the IgM Fc region includes the Cμ3 domain, the Cμ4 domain, and the IgM tailpiece.
64. The multi-domain binding molecule of embodiment 55, wherein the multimerizing fragment of the IgM Fc region includes the Cμ2 domain, the Cμ3 domain, the Cμ4 domain, and the IgM tailpiece.
65. The multi-domain binding molecule of embodiment 55, wherein the multimerizing fragment of the IgM Fc region includes the Cμ1 domain, the Cμ2 domain, the Cμ3 domain, the Cμ4 domain, and the IgM tailpiece.
66. The multi-domain binding molecule of embodiment 55, wherein the multimerizing fragment of the IgM Fc region has the sequence as set forth in SEQ ID NO: 125.
67. A single-chain variable fragment (scFv) including an antibody fragment of any of embodiments 1-39, wherein the antibody fragment includes a humanized light chain variable region and/or a humanized heavy chain variable region and lacks a constant region.
68. A conjugate including the antibody or fragment thereof of any of embodiments 1-39 linked to a radioactive isotope, an immunotoxin, a drug, a detectable label, or a particle.
69. The conjugate of embodiment 68, wherein the radioactive isotope includes ²²⁸Ac, ¹¹¹Ag, ¹²⁴Am, ⁷⁴As, ²¹¹At, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ¹⁴C ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ³H, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb ¹⁷⁵Yb, ⁶⁵Zn, ⁷¹mZn, ⁸⁶Zr, ⁹⁵Zr, or ⁹⁷Zr.
70. The conjugate of embodiments 68 or 69, wherein the radioactive isotope includes ¹³¹I, ⁹⁰Y, or ²¹¹At.
71. The conjugate of any of embodiments 68-70, wherein the radioactive isotope includes a half-life of 7.2 hours.
72. The conjugate of any of embodiments 68-71, wherein the radioactive isotope does not emit daughter radionuclides.
73. The conjugate of embodiment 68, wherein the immunotoxin includes a plant toxin or bacterial toxin.
74. The conjugate of embodiment 73, wherein the plant toxin includes ricin, abrin, mistletoe lectin, modeccin, pokeweed antiviral protein, saporin, Bryodin 1, bouganin, or gelonin.
75. The conjugate of embodiment 73, wherein the bacterial toxin includes diphtheria toxin or Pseudomonas exotoxin.
76. The conjugate of embodiment 68, wherein the drug includes a cytotoxic drug.
77. The conjugate of embodiment 76, wherein the cytotoxic drug includes actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine, taxane, taxol, tenoposide, tetracaine, trichothecene, vinblastine, vinca alkaloid, or vincristine.
78. The conjugate of embodiment 68, wherein the detectable label includes a fluorescent label, a chemiluminescent label, a spectral colorimetric label, an enzymatic label, or an affinity tag.
79. The conjugate of embodiment 78, wherein the fluorescent label includes blue fluorescent protein, cyan fluorescent protein, green fluorescent protein, luciferase, orange fluorescent protein, red fluorescent protein, far red fluorescent protein, or yellow fluorescent protein.
80. The conjugate of embodiment 78, wherein the chemiluminescent label includes lucigenin, luminol, luciferin, isoluminol, theromatic acridinium ester, imidazole, acridinium salt, or oxalate ester.
81. The conjugate of embodiment 78, wherein the spectral colorimetric label includes colloidal gold.
82. The conjugate of embodiment 78, wherein the enzymatic label includes malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase, or acetylcholinesterase.
83. The conjugate of embodiment 78, wherein the affinity tag includes a tag with a sequence as set forth in SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, or SEQ ID NO: 152.
84. A chimeric antigen receptor (CAR) that, when expressed by a cell, includes an extracellular component linked to an intracellular component by a transmembrane domain, wherein the extracellular component includes the scFv of embodiment 67.
85. The CAR of embodiment 84, wherein the intracellular component includes an effector domain including: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or a combination thereof.
86. The CAR of embodiments 84 or 85, wherein the transmembrane domain includes a transmembrane region of: the α, β or ζ chain of a T-cell receptor; CD28; CD27; CD3; CD45; CD4; CD5; CD8; CD9; CD16; CD22; CD33; CD37; CD64; CD80; CD86; CD134; CD137; CD154; or a combination thereof.
87. The CAR of any of embodiments 84-86, wherein the CAR further includes a spacer region.
88. An engineered T cell receptor (eTCR) including a constant alpha domain (C_{α}), a constant beta domain (C_{β}), and the scFv of embodiment 67 linked to the C_{α} domain and/or the C_{β} domain.
89. The eTCR of embodiment 88, wherein the scFv of embodiment 67 is linked to the C_{α} domain.
90. The eTCR of embodiments 88 or 89, wherein the scFv of embodiment 67 is linked to the C_{β} domain.
91. The eTCR of any of embodiments 88-90, wherein one scFv of embodiment 67 is linked to the C_{α} domain and one scFv of embodiment 67 is linked to the C_{β} domain.
92. A nucleic acid encoding an antibody or fragment thereof of any of embodiments 1-39.
93. The nucleic acid of embodiment 92, wherein the nucleic acid has the sequence as set forth in SEQ ID NOs: 14, 15, 16, 17, 18, 19, 20, 21, or 22, or has a sequence with at least 90% sequence identity to the sequence as set forth in SEQ ID NOs: 14, 15, 16, 17, 18, 19, 20, 21, or 22 wherein the encoded antibody or fragment thereof retains CD45 binding.
94. A cell genetically modified to express the antibody or fragment thereof of any of embodiments 1-39, the CAR of any of embodiments 84-87, or the eTCR of any of embodiments 88-91.
95. The cell of embodiment 94, wherein the cell is an immune cell.
96. The cell of embodiment 95, wherein the immune cell is a T cell, B cell, natural killer cell, or macrophage.
97. A composition including the antibody or fragment thereof of any of embodiments 1-39 or a nucleic acid of embodiments 92 or 93, and a pharmaceutically acceptable carrier.
98. A formulation including the cell of any of embodiments 94-96 and a pharmaceutically acceptable carrier.
99. A kit including the antibody or fragment thereof of any of embodiments 1-39, the multi-domain binding molecule of any of embodiments 40-66, the conjugate of any of embodiments 68-83, the CAR of any of embodiments 84-87, the eTCR of any of embodiments 88-91, or the nucleic acid of embodiments 92 or 93.
100. A method of treating a subject in need thereof including administering a therapeutically effective amount of the composition of embodiment 97 and/or the formulation of embodiment 98 thereby treating the subject in need thereof.
101. The method of embodiment 100, wherein the therapeutically effective amount provides a prophylactic or a therapeutic treatment against a hematologic disorder.
102. The method of embodiment 101, wherein the hematologic disorder comprises a hematopoietic malignancy.
103. The method of embodiment 102, wherein the hematologic malignancy includes acute myelogenous leukemia (AML), acute lymphocytic leukemia (ALL), myelodysplastic syndromes with excess blasts, chronic lymphocytic leukemia (CLL), or chronic myelogenous leukemia (CML).
104. The method of embodiment 103, wherein the AML includes relapsed or refractory AML.
105. The method of any of embodiments 100-104, wherein the therapeutically effective amount provides a conditioning treatment for a subject before the subject receives a hematopoietic cell transplant.
106. The method of embodiment 105, wherein the hematopoietic cell transplant includes allogeneic hematopoietic cell transplant or autologous hematopoietic cell transplant.
107. The method of embodiment 106, wherein the transplanted hematopoietic cell are gene-edited.
108. The method of any of embodiments 100-107, wherein the subject is a human subject with an autoimmune disease, a metabolic disorder, or an inherited blood disorder.
109. The method of any of embodiments 100-108, wherein the administering is through intravenous, intradermal, intraarterial, intranodal, intravesicular, intrathecal, intraperitoneal, intraparenteral, intranasal, intralesional, intramuscular, oral, intrapulmonary, subcutaneous, or sublingual administering.

### (XII) Experimental Examples. Development of Humanized BC8 as Basis for Improved CD45-Directed Immunotherapy

Background and Significance. (A) CD45 as therapeutic drug target. CD45, also known as leukocyte common antigen, is a transmembrane cell surface glycoprotein (molecular weight: 180-220 kDa) with tyrosine phosphatase activity. Dahlke et al., Leuk Lymphoma. 2004; 45(2):229-236. Absent on non-hematopoietic cells, CD45 is expressed on almost all hematopoietic cells. Exceptions are mature thrombocytes, mature erythrocytes, and some of their progenitors. Consistent with this broad expression on cells of the blood system, most hematologic malignancies, including 85-90% of cases of acute myeloid leukemia (AML) and acute lymphoblastic leukemia (ALL), express CD45. Dahlke et al., Leuk Lymphoma. 2004; 45(2):229-236. This expression pattern has raised long-standing interest in CD45 as a target for antigen-specific immunotherapy. Most efforts to date have centered on CD45-directed therapies to treat hematologic malignancies. However, other applications have been explored or envisioned, including the use as targeted lymphodepletion as part of a conditioning regimen prior to adoptive cell therapy (Dawicki et al., Oncotarget. 2020; 11(39):3571-3581) or to facilitate engraftment of normal or gene edited hematopoietic stem/progenitor cells in patients with non-malignant disorders undergoing hematopoietic cell transplantation (HCT). Gao et al., Blood Adv. 2019; 3(18):2700-2711; Srikanthan et al., Mol Ther Methods Clin Dev. 2020; 17:455-464; Castiello et al., J Allergy Clin Immunol. 2021; 147(1):309-320 e306.

To date, radiolabeled antibodies (radioimmunotherapy [RIT]) have been a major focus of efforts with CD45-targeted immunotherapies. Ideal for RIT applications, CD45 is displayed at a relatively high copy number (200,000/cell). It is relatively stable on the cell surface (although CD45-directed immunotoxins (Castiello et al., J Allergy Clin Immunol. 2021; 147(1):309-320 e306) and antibody-drug conjugates (Gao et al., Blood Adv. 2019; 3(18):2700-2711; Srikanthan et al., Mol Ther Methods Clin Dev. 2020; 17:455-464; Persaud et al., J Clin Invest. 2021; 131(24)) have started to be explored very recently), and there is no appreciable shedding after ligand binding. Press et al., Blood. 1994; 83(5):1390-1397. Of note, while several isoforms of CD45 exist, the antibodies used so far as basis for CD45-directed therapeutics recognize all isoforms, i.e. they are pan-specific. Dahlke et al., Leuk Lymphoma. 2004; 45(2):229-236.

(B) CD45-directed RIT to improve outcomes with hematopoietic cell transplantation (HCT) for acute leukemia and myelodysplastic neoplasms (MDS). While the broad expression of CD45 renders CD45-directed therapies useful for a wide variety of hematologic malignancies, the use of CD45-directed RIT is so far best studied in patients with acute leukemia and MDS. For these malignancies, allogeneic HCT remains a pivotal cornerstone of treatment despite recent approval of several new drugs. Döhner et al., Nat Rev Clin Oncol. 2021; 18(9):577-590; Döhner et al., Blood. 2022; 140(12):1345-1377; Shimony et al., Am J Hematol. 2023; 98(3):502-526; DiNardo et al., Acute myeloid leukaemia. Lancet. 2023; in press; Giebel et al., Bone Marrow Transplant. 2019; 54(6):798-809; DeFilipp et al., Biol Blood Marrow Transplant. 2019; 25(11):2113-2123; Calvo et al., Front Pediatr. 2021; 9:796426; Platzbecker, Blood. 2019; 133(10):1096-1107; Vittayawacharin et al., Am J Hematol. 2023; 98(2):322-337; DeFilipp et al., Transplant Cell Ther. 2023; 29(2):71-81. Still, relapses are common and constitute the leading cause of morbidity and mortality after HCT. Toxicities related to conditioning therapy further curtail the HCT success. Hence, there is great need to improve the efficacy and safety of this treatment modality. Because of the exquisite radiosensitivity of neoplastic and normal hematopoietic cells, radiolabeled monoclonal antibodies (mAbs) have long been pursued for this purpose. This radiosensitivity is clinically exploitable as demonstrated by the benefit of total body irradiation (TBI) with HCT. Randomized trials, for example in patients with AML or chronic myeloid leukemia (CML), showed this effect is dose-dependent but reduced relapses did not yield longer survival due to higher non-relapse mortality from toxicities to normal organs. Clift et al., Blood. 1990; 76(9):1867-1871; Clift et al., Blood. 1998; 92(4):1455-1456; Clift et al., Blood. 1991; 77(8):1660-1665. This observation provided the rationale to employ anti-CD45 mAbs to target radiation to neoplastic hematopoietic cells.

Efforts with CD45-directed RIT to date have largely aimed at strategies to augment transplant conditioning regimens before allogeneic HCT via delivery of radiation to marrow, spleen, and lymph nodes in patients with hematologic malignancies with high or low tumor burden. Ali et al., Blood Rev. 2016; 30(5):389-399. Proof-of-principle for the idea of using CD45-directed RIT to treat such patients was demonstrated with β-emitters such as iodine-131 (¹³¹I) or yttrium-90 (⁹⁰Y). When coupled to a murine anti-CD45 IgG1 mAb (clone BC8), 2-to-3-fold higher radiation doses were delivered to spleen and bone marrow than any critical normal organ together with high- or reduced-intensity conditioning in patients with acute leukemias or MDS. Pagel et al., Blood. 2006; 107(5):2184-2191; Pagel et al., Blood. 2009; 114(27):5444-5453; Vo et al., Haematologica. 2020; 105(6):1731-1737. Following up on these early results, ¹³¹I-BC8 (a.k.a. ¹³¹I-apamistamab or Iomab-B) combined with non-myeloablative allogeneic HCT has recently shown to yield better outcomes than conventional care in a randomized trial (SIERRA; NCT02665065) in 153 older adults with relapsed or refractory acute myeloid leukemia treated at 24 academic sites in the U.S. and Canada (AML; late breaking abstract at 2023 Tandem Meeting of ASTCT and CIBMTR).

In parallel to the late-phase testing of β-emitter RIT targeting CD45, there is increasing interest in using α-emitters such as astatine-211 (²¹¹At) as a payload for anti-CD45 mAbs. α-emitters deposit a higher decay energy (5-8 MeV) over shorter distances (55-70 μm) for potent, precise, and efficient kill of target cells or small clusters of cells and minimized toxicity to normal, non-targeted surrounding cells compared to β-emitters, which deliver lower decay energies (0.66-2.3 MeV) over longer path lengths (0.3-2.3 mm). Behr et al., Cancer Res. 1999; 59(11):2635-2643; Larson et al., Nat Rev Cancer. 2015; 15(6):347-360; Aghevlian et al., Adv Drug Deliv Rev. 2017; 109:102-118. For several α-emitters, including ²¹¹At, it has been shown that as few as 10 hits or less per cell kill hematopoietic neoplasms. Nikula et al., J Nucl Med. 1999; 40(1):166-176; Sawant et al., Radiat Res. 2001; 156(2):177-180; Aurlien et al., Int J Radiat Biol. 2002; 78(2):133-142; Dahle et al., Blood. 2007; 110(6):2049-2056. While other α-emitters have been pursued, (Sandmaier et al., Blood. 2002; 100(1):318-326; Bethge et al., Transplantation. 2004; 78(3):352-359; Nakamae et al., Cancer Res. 2009; 69(6):2408-2415)²¹¹At is particularly useful for the clinic because of its half-life (t_{1/2}) of 7.2 hours (enabling high-yield radiolabeling and easy drug delivery) and because it does not release α-emitting daughter radionuclides that could cause organ toxicity. Aghevlian et al., Adv Drug Deliv Rev. 2017; 109:102-118. In animal models, ²¹¹At-labeled anti-CD45 mAbs were highly efficacious against acute leukemia, B-cell lymphoma, and multiple myeloma in vivo, including minimal residual disease (MRD) burdens. Orozco et al., Blood. 2013; 121(18):3759-3767. As translation, 2 first-in-human trials testing ²¹¹At-labeled BC8 combined with fludarabine/2-3 Gy TBI before HCT were initiated for adults with acute leukemia or MDS with HLA-matched related or unrelated donors (NCT3128034) and, more recently, HLA-haploidentical donors (NCT03670966). Data from the first 20 patients treated on the former indicate that doses of ²¹¹At up to 500 μCi/kg can be safely delivered with the murine anti-CD45 mAb antibody in combination with nonmyeloablative conditioning. Sandmaier et al., Transplant Cell Ther. 2021; 27(Suppl 3):S54.

(C) Humanized BC8 to overcome limitations of murine BC8. The clinical experience summarized above establishes BC8 as promising therapeutic. However, because of the murine nature of BC8, many patients experience significant infusion toxicities. Moreover, human anti-mouse antibody (HAMA) responses can occur, even after just a single BC8 infusion for dosimetry purposes, (Pagel et al., Blood. 2009; 114(27):5444-5453; Matthews et al., Blood. 1999; 94(4):1237-1247) which precludes future use of any murine mAb. Another murine mAb, M195 (anti-CD33 mAb) resulted in HAMA in 37% of patients, (Schwartz et al., J Clin Oncol. 1993; 11(2):294-303) spurring mAb humanization for further clinical use. Co et al., J Immunol. 1992; 148(4):1149-1154. Development of a HAMA response limits future use of any murine mAb-based therapy. By minimizing infusion toxicities and risks of HAMA reactions, humanization of BC8 (HuBC8) overcomes these current limitations of BC8 and form the basis for improved CD45− targeted immunotherapeutics which, unlike drugs built on murine BC8, would allow multi-dosing with the same mAb.

(D) Experimental Description. BC8 is a murine IgG1 kappa monoclonal antibody specific for human (and nonhuman primate) CD45 originally developed at Fred Hutchinson Cancer Center (FHCC). The full sequence of the light and heavy chain variable regions of BC8 have been reported both by investigators at FHCC in the peer-reviewed literature (Lin et al., Cancer Res. 2006; 66(7):3884-3892) and, more recently, in the patent literature (U.S. Pat. No. 5,273,738 and WO2017155937A1). The BC8 hybridoma cell line was used to generate mRNA to independently validate sequences of BC8 variable regions utilizing 5′ RACE cloning with isotype-specific PCR primers. Humanization was accomplished by grafting the complementarity-determining regions (CDRs) of BC8 into the human variable domain germline heavy and light chain sequences with highest homology to the BC8 heavy and light chain sequences. The closest human light chain gene was identified to be IGKV7-3 but, as a pseudogene, was not used for grafting purposes. The next closest matches were IGKV1-39 and IGKV4-1; ultimately, IGKV4-1 was chosen as the most appropriate backbone for CDR grafting due to its longer CDR1 and similarity to BC8 in the framework regions. IGHV3-74 was identified as the closest human heavy chain framework and was used for CDR grafting purposes. Two variants of humanized BC8 were generated. One variant was built using murine CDRs (CDRL1: RASKSVSTSGYSYLH (SEQ ID NO: 37); CDRL2: LASNLES (SEQ ID NO: 46); CDRL3: QHSRELPFT (SEQ ID NO: 39); CDRH1: GFDFSRYWMS (SEQ ID NO: 47); CDRH2: EINPTSSTINFTPSLKD (SEQ ID NO: 48); CDRH3: GNYYRYGDAMDY (SEQ ID NO: 49)), human variable chain framework regions, and human constant regions (bare-grafted HuBC8). For the kappa light chain, this yielded 80/111 (72.1%) amino acids of human origin in the variable region and 187/218 (85.8%) amino acids of human origin in the entire light chain; for the heavy chain, this yielded 82/121 (67.8%) amino acids of human origin in the variable region and 412/451 (91.4%) and 409/448 (91.3%) amino acids of human origin in the entire IgG1 and IgG4 heavy chain, respectively. A second variant was built that included, in addition to the murine CDRs, murine residues at positions 3 and 85 within the human light chain variable region and murine residues at positions 46, 48, 49, and 66-68 within the human heavy chain variable region (CDR-grafted HuBC8 with back mutations). For the kappa light chain, this yielded 78/111 (70.3%) amino acids of human origin in the variable region and 185/218 (84.9%) amino acids of human origin in the entire light chain; for the heavy chain, this yielded 76/121 (62.8%) amino acids of human origin in the variable region and 406/451 (90.0%) and 403/448 (90.0%) amino acids of human origin in the entire IgG1 and IgG4 heavy chain, respectively.

(XIII) Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, lie, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a humanized anti-CD45 antibody) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{of}f) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N. Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant increase in binding affinity for CD45 by a chimeric or humanized antibody disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An antibody or fragment thereof that binds CD45 comprising a light chain variable region comprises a complementarity determining region (CDR) light (L)1, CDRL2, and CDRL3 and a heavy chain variable region comprises a CDR heavy (H)1, CDRH2, and CDRH3;
wherein the CDRL1 comprises the sequence as set forth in SEQ ID NO: 37, the CDRL2 comprises the sequence as set forth in SEQ ID NO: 38, the CDRL3 comprises the sequence as set forth in SEQ ID NO: 39, the CDRH1 comprises the sequence as set forth in SEQ ID NO: 40, the CDRH2 comprises the sequence as set forth in SEQ ID NO: 41, and the CDRH3 comprises the sequence as set forth in SEQ ID NO: 42 according to North;
wherein the CDRL1 comprises the sequence as set forth in SEQ ID NO: 43, the CDRL2 comprises the sequence comprising LAS, the CDRL3 comprises the sequence as set forth in SEQ ID NO: 39, the CDRH1 comprises the sequence as set forth in SEQ ID NO: 44, the CDRH2 comprises the sequence as set forth in SEQ ID NO: 45 and the CDRH3 comprises the sequence as set forth in SEQ ID NO: 42 according to IMGT;
wherein the CDRL1 comprises the sequence as set forth in SEQ ID NO: 37, the CDRL2 comprises the sequence as set forth in SEQ ID NO: 46, the CDRL3 comprises the sequence as set forth in SEQ ID NO: 39, the CDRH1 comprises the sequence as set forth in SEQ ID NO: 47, the CDRH2 comprises the sequence as set forth in SEQ ID NO: 48, and the CDRH3 comprises the sequence as set forth in SEQ ID NO: 49 according to Kabat;
wherein the CDRL1 comprises the sequence as set forth in SEQ ID NO: 37, the CDRL2 comprises the sequence as set forth in SEQ ID NO: 46, the CDRL3 comprises the sequence as set forth in SEQ ID NO: 39, the CDRH1 comprises the sequence as set forth in SEQ ID NO: 50, the CDRH2 comprises the sequence as set forth in SEQ ID NO: 51, and the CDRH3 comprises the sequence as set forth in SEQ ID NO: 49 according to Chothia;
wherein the CDRL1 comprises the sequence as set forth in SEQ ID NO: 52, the CDRL2 comprises the sequence as set forth in SEQ ID NO: 53, the CDRL3 comprises the sequence as set forth in SEQ ID NO: 54, the CDRH1 comprises the sequence as set forth in SEQ ID NO: 55, the CDRH2 comprises the sequence as set forth in SEQ ID NO: 56, and the CDRH3 comprises the sequence as set forth in SEQ ID NO: 57 according to Contact; or
wherein CDRL1 comprises the sequence as set forth in SEQ ID NO: 52, CDRL2 comprises the sequence as set forth in SEQ ID NO: 53, CDRL3 comprises the sequence as set forth in SEQ ID NO: 54, CDRH1 comprises the sequence as set forth in SEQ ID NO: 55, CDRH2 comprises the sequence as set forth in SEQ ID NO: 58, and CDRH3 comprises the sequence as set forth in SEQ ID NO: 57 according to Contact,
wherein the antibody or fragment thereof further comprises human variable framework regions and/or a human constant region.

2. The antibody or fragment thereof of claim 1, wherein the light chain variable region comprises a murine BC8 light chain variable region, a humanized anti-CD45 (CDR HuBC8) light chain variable region, or a humanized anti-CD45 with back mutations (CDR/BM HuBC8) light chain variable region.

3. The antibody or fragment thereof of claim 2, wherein the murine BC8 light chain variable region comprises the sequence as set forth in SEQ ID NO: 59 or a sequence having at least 90% sequence identity to SEQ ID NO: 59, wherein the antibody or fragment thereof retains CD45 binding.

4. The antibody or fragment thereof of claim 2, wherein the CDR HuBC8 light chain variable region comprises the sequence as set forth in SEQ ID NO: 61 or a sequence having at least 90% sequence identity to SEQ ID NO: 61, wherein the antibody or fragment thereof retains CD45 binding.

5. The antibody or fragment thereof of claim 2, wherein the CDR/BM HuBC8 light chain variable region comprises the sequence as set forth in SEQ ID NO: 63 or a sequence having at least 90% sequence identity to SEQ ID NO: 63, wherein the antibody or fragment thereof retains CD45 binding.

6. The antibody or fragment thereof of claim 1, wherein the heavy chain variable region comprises a murine BC8 heavy chain variable region, a CDR HuBC8 heavy chain variable region, or a CDR/BM HuBC8 heavy chain variable region.

7. The antibody or fragment thereof of claim 6, wherein the murine BC8 heavy chain variable region comprises the sequence as set forth in SEQ ID NO: 60 or a sequence having at least 90% sequence identity to SEQ ID NO: 60, wherein the antibody or fragment thereof retains CD45 binding.

8. The antibody or fragment thereof of claim 6, wherein the CDR HuBC8 chain variable region comprises the sequence as set forth in SEQ ID NO: 62 or a sequence having at least 90% sequence identity to SEQ ID NO: 62, wherein the antibody or fragment thereof retains CD45 binding.

9. The antibody or fragment thereof of claim 6, wherein the CDR/BM HuBC8 heavy chain variable region comprises the sequence as set forth in SEQ ID NO: 64 or a sequence having at least 90% sequence identity to SEQ ID NO: 64, wherein the antibody or fragment thereof retains CD45 binding.

10. The antibody or fragment thereof of claim 1, wherein the antibody or fragment thereof further comprises the human constant region.

11. The antibody or fragment thereof of claim 10, wherein the human constant region comprises a human light chain constant region and/or a human heavy chain constant region.

12. The antibody or fragment thereof of claim 11, wherein the human light chain constant region comprises a human Igκ light chain constant region or a human IgA light chain constant region.

13. The antibody or fragment thereof of claim 11, wherein the human light chain constant region comprises a human Igκ light chain constant region.

14. The antibody or fragment thereof of claim 12, wherein the human Igκ light chain constant region comprises the sequence as set forth in SEQ ID NO: 26.

15. The antibody or fragment thereof of claim 11, wherein the human heavy chain constant region comprises a human IgG1 heavy chain constant region, a human IgG4_S228P heavy chain constant region, a human IgG4 heavy chain constant region, a human IgG2 heavy chain constant region, or a human IgG3 heavy chain constant region.

16. The antibody or fragment thereof of claim 11, wherein the human heavy chain constant region comprises a human IgG1 heavy chain constant region or a human IgG4_S228P heavy chain constant region.

17. The antibody or fragment thereof of claim 15, wherein the human IgG1 heavy chain constant region comprises the sequence as set forth in SEQ ID NO: 28.

18. The antibody or fragment thereof of claim 15, wherein the human IgG4_S228P heavy chain constant region comprises the sequence as set forth in SEQ ID NO: 36.

19. The antibody or fragment thereof of claim 1, wherein the light chain variable region further comprises a signal peptide.

20. The antibody or fragment thereof of claim 1, wherein the heavy chain variable region further comprises a signal peptide.

21. The antibody or fragment thereof of claim 19 or 20, wherein the signal peptide comprises a sequence as set forth in SEQ ID NO: 1, SEQ ID NO: 2, or SEQ ID NO: 3.

22. The antibody or fragment thereof of claim 1, wherein the light chain variable region comprises a murine BC8 light chain variable region and wherein the antibody or fragment thereof comprises a human Igκ light chain constant region.

23. The antibody or fragment thereof of claim 22, comprising the sequence as set forth in SEQ ID NO: 4.

24. The antibody or fragment thereof of claim 1, wherein the heavy chain variable region comprises a murine BC8 heavy chain variable region and wherein the antibody or fragment thereof comprises a human IgG1 heavy chain constant region.

25. The antibody or fragment thereof of claim 24, comprising the sequence as set forth in SEQ ID NO: 5 or a sequence having at least 90% sequence identity to SEQ ID NO: 5, wherein the antibody or fragment thereof retains CD45 binding.

26. The antibody or fragment thereof of claim 1, wherein the heavy chain variable region comprises a murine BC8 heavy chain variable region and wherein the antibody or fragment thereof comprises a human IgG4_S228P heavy chain constant region.

27. The antibody or fragment thereof of claim 26, comprising the sequence as set forth in SEQ ID NO: 6 or a sequence having at least 90% sequence identity to SEQ ID NO: 6, wherein the antibody or fragment thereof retains CD45 binding.

28. The antibody or fragment thereof of claim 1, wherein the light chain variable region comprises a CDR HuBC8 light chain variable region and wherein the antibody or fragment thereof comprises a human Igκ light chain constant region.

29. The antibody or fragment thereof of claim 28, comprising the sequence as set forth in SEQ ID NO: 7 or a sequence having at least 90% sequence identity to SEQ ID NO: 7, wherein the antibody or fragment thereof retains CD45 binding.

30. The antibody or fragment thereof of claim 1, wherein the heavy chain variable region comprises a CDR HuBC8 heavy chain variable region and wherein the antibody or fragment thereof comprises a human IgG1 heavy chain constant region.

31. The antibody or fragment thereof of claim 30, comprising the sequence as set forth in SEQ ID NO: 8 or a sequence having at least 90% sequence identity to SEQ ID NO: 8, wherein the antibody or fragment thereof retains CD45 binding.

32. The antibody or fragment thereof of claim 1, wherein the heavy chain variable region comprises a CDR HuBC8 heavy chain variable region and wherein the antibody or fragment thereof comprises a human IgG4_S228P heavy chain constant region.

33. The antibody or fragment thereof of claim 32, comprising the sequence as set forth in SEQ ID NO: 9 or a sequence having at least 90% sequence identity to SEQ ID NO: 9, wherein the antibody or fragment thereof retains CD45 binding.

34. The antibody or fragment thereof of claim 1, wherein the light chain variable region comprises a CDR/BM HuBC8 light chain variable region and wherein the antibody or fragment thereof comprises a human Igκ light chain constant region.

35. The antibody or fragment thereof of claim 34, comprising the sequence as set forth in SEQ ID NO: 10 or a sequence having at least 90% sequence identity to SEQ ID NO: 10, wherein the antibody or fragment thereof retains CD45 binding.

36. The antibody or fragment thereof of claim 1, wherein the heavy chain variable region comprises a CDR/BM HuBC8 heavy chain variable region and wherein the antibody or fragment thereof comprises a human IgG1 heavy chain constant region.

37. The antibody or fragment thereof of claim 36, comprising the sequence as set forth in SEQ ID NO: 11 or a sequence having at least 90% sequence identity to SEQ ID NO: 11, wherein the antibody or fragment thereof retains CD45 binding.

38. The antibody or fragment thereof of claim 1, wherein the heavy chain variable region comprises a CDR/BM HuBC8 heavy chain variable region and wherein the antibody or fragment thereof comprises a human IgG4_S228P heavy chain constant region.

39. The antibody or fragment thereof of claim 38, comprising the sequence as set forth in SEQ ID NO: 12 or a sequence having at least 90% sequence identity to SEQ ID NO: 12, wherein the antibody or fragment thereof retains CD45 binding.

40. A multi-domain binding molecule comprising at least two binding domains wherein at least one binding domain comprises the antibody or fragment thereof of claim 1.

41. The multi-domain binding molecule of claim 40, wherein the multi-domain binding molecule comprises an immune cell engaging molecule.

42. The multi-domain binding molecule of claim 41, wherein the immune cell engaging molecule activates a B cell, T cell, natural killer (NK) cell, or macrophage.

43. The multi-domain binding molecule of claim 42, wherein the T cell is a CD3 T cell, a CD4 T cell, a CD8 T cell, a central memory T cell, an effector memory T cell, and/or a naïve T cell.

44. The multi-domain binding molecule of claim 41, wherein a binding domain of the immune cell engaging molecule binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Ra, or MARCO.

45. The multi-domain binding molecule of claim 40, wherein the at least two binding domains comprise at least two copies of the antibody or fragment thereof of claim 1.

46. The multi-domain binding molecule of claim 45, wherein the at least two copies are joined by a protein linker.

47. The multi-domain binding molecule of claim 46, wherein the protein linker is a Gly-Ser linker.

48. The multi-domain binding molecule of claim 47, wherein the Gly-Ser linker is (GlyₓSer_{y})ₙ wherein x and y are independently an integer from 0 to 10 provided that x and y are not both 0 and wherein n is an integer of 1, 2, 3, 4, 5, 6, 7, 8, 9 or 10.

49. The multi-domain binding molecule of claim 40, comprising 2, 3, 4, 5, 6, 7, 8, 9, or 10 copies of the antibody or fragment thereof of claim 1.

50. The multi-domain binding molecule of claim 40, wherein the multi-domain binding molecule is a dimer, trimer, tetramer, pentamer, hexamer, or heptamer.

51. The multi-domain binding molecule of claim 46, wherein the at least two copies are linked to an Fc region of an antibody.

52. The multi-domain binding molecule of claim 51, wherein the Fc region is an IgA Fc region or an IgM Fc region.

53. The multi-domain binding molecule of claim 51, wherein the Fc region is an IgA Fc region having the sequence as set forth in SEQ ID NOs: 117 and 118.

54. The multi-domain binding molecule of claim 51, wherein the Fc region is an IgM Fc region having the sequence as set forth in SEQ ID NOs: 122-132.

55. The multi-domain binding molecule of claim 51, wherein the Fc region comprises a multimerizing fragment of the IgA Fc region or a multimerizing fragment of the IgM Fc region.

56. The multi-domain binding molecule of claim 55, wherein the multimerizing fragment of the Igλ Fc region comprises the IgA tailpiece.

57. The multi-domain binding molecule of claim 56, wherein the IgA tailpiece has the sequence of residues 331-352 as set forth in SEQ ID NO: 117 or the sequence of residues 318-340 as set forth in SEQ ID NO: 118.

58. The multi-domain binding molecule of claim 55, wherein the multimerizing fragment of the Igλ Fc region comprises the IgA CA3 domain and the IgA tailpiece.

59. The multi-domain binding molecule of claim 55, wherein the multimerizing fragment of the Igλ Fc region comprises the IgA CA2 domain, the IgA CA3 domain, and the IgA tailpiece.

60. The multi-domain binding molecule of claim 55, wherein the multimerizing fragment of the Igλ Fc region comprises the IgA CA1 domain, the IgA CA2 domain, the IgA CA3 domain, and the Igλ tailpiece.

61. The multi-domain binding molecule of claim 55, wherein the multimerizing fragment of the IgM Fc region comprises the IgM tailpiece.

62. The multi-domain binding molecule of claim 55, wherein the multimerizing fragment of the IgM Fc region comprises the Cμ4 domain and the IgM tailpiece.

63. The multi-domain binding molecule of claim 55, wherein the multimerizing fragment of the IgM Fc region comprises the Cμ3 domain, the Cμ4 domain, and the IgM tailpiece.

64. The multi-domain binding molecule of claim 55, wherein the multimerizing fragment of the IgM Fc region comprises the Cμ2 domain, the Cμ3 domain, the Cμ4 domain, and the IgM tailpiece.

65. The multi-domain binding molecule of claim 55, wherein the multimerizing fragment of the IgM Fc region comprises the Cμ1 domain, the Cμ2 domain, the Cμ3 domain, the Cμ4 domain, and the IgM tailpiece.

66. The multi-domain binding molecule of claim 55, wherein the multimerizing fragment of the IgM Fc region has the sequence as set forth in SEQ ID NO: 125.

67. A single-chain variable fragment (scFv) comprising an antibody fragment of claim 1, wherein the antibody fragment comprises a humanized light chain variable region and/or a humanized heavy chain variable region and lacks a constant region.

68. A conjugate comprising the antibody or fragment thereof of claim 1 linked to a radioactive isotope, an immunotoxin, a drug, a detectable label, or a particle.

69. The conjugate of claim 68, wherein the radioactive isotope comprises ²²⁸Ac, ¹¹¹Ag, ¹²⁴Am, ⁷⁴As, ²¹¹At, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ¹⁴C ⁴⁷Ca, ²⁵⁴Cf, ²⁴²Cm ⁵¹Cr ⁶⁷CU ¹⁵³Dy, ¹⁵⁷Dy, ¹⁵⁹Dy, ¹⁶⁵Dy, ¹⁶⁶Dy, ¹⁷¹Er, ²⁵⁰Es ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ³H, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹ Rn, ¹⁰³Ru, ³⁵S, ⁴⁴Sc, ⁷²Se ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb ⁶⁵Zn, ⁷¹mZn, ⁸⁶Zr, ⁹⁵Zr, or ⁹⁷Zr.

70. The conjugate of claim 68, wherein the radioactive isotope comprises ¹³¹I, ⁹⁰Y, or ²¹¹At.

71. The conjugate of claim 68, wherein the radioactive isotope comprises a half-life of 7.2 hours.

72. The conjugate of claim 68, wherein the radioactive isotope does not emit daughter radionuclides.

73. The conjugate of claim 68, wherein the immunotoxin comprises a plant toxin or bacterial toxin.

74. The conjugate of claim 73, wherein the plant toxin comprises ricin, abrin, mistletoe lectin, modeccin, pokeweed antiviral protein, saporin, Bryodin 1, bouganin, orgelonin.

75. The conjugate of claim 73, wherein the bacterial toxin comprises diphtheria toxin or Pseudomonas exotoxin.

76. The conjugate of claim 68, wherein the drug comprises a cytotoxic drug.

77. The conjugate of claim 76, wherein the cytotoxic drug comprises actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine, taxane, taxol, tenoposide, tetracaine, trichothecene, vinblastine, vinca alkaloid, or vincristine.

78. The conjugate of claim 68, wherein the detectable label comprises a fluorescent label, a chemiluminescent label, a spectral colorimetric label, an enzymatic label, or an affinity tag.

79. The conjugate of claim 78, wherein the fluorescent label comprises blue fluorescent protein, cyan fluorescent protein, green fluorescent protein, luciferase, orange fluorescent protein, red fluorescent protein, far red fluorescent protein, or yellow fluorescent protein.

80. The conjugate of claim 78, wherein the chemiluminescent label comprises lucigenin, luminol, luciferin, isoluminol, theromatic acridinium ester, imidazole, acridinium salt, or oxalate ester.

81. The conjugate of claim 78, wherein the spectral colorimetric label comprises colloidal gold.

82. The conjugate of claim 78, wherein the enzymatic label comprises malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase, or acetylcholinesterase.

83. The conjugate of claim 78, wherein the affinity tag comprises a tag with a sequence as set forth in SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, or SEQ ID NO: 152.

84. A chimeric antigen receptor (CAR) that, when expressed by a cell, comprises an extracellular component linked to an intracellular component by a transmembrane domain, wherein the extracellular component comprises the scFv of claim 67.

85. The CAR of claim 84, wherein the intracellular component comprises an effector domain comprising: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or a combination thereof.

86. The CAR of claim 84, wherein the transmembrane domain comprises a transmembrane region of: the α, β or ζ chain of a T-cell receptor; CD28; CD27; CD3; CD45; CD4; CD5; CD8; CD9; CD16; CD22; CD33; CD37; CD64; CD80; CD86; CD134; CD137; CD154; or a combination thereof.

87. The CAR of claim 84, wherein the CAR further comprises a spacer region.

88. An engineered T cell receptor (eTCR) comprising a constant alpha domain (C_{α}), a constant beta domain (C_{β}), and the scFv of claim 67 linked to the C_{α} domain and/or the C_{β} domain.

89. The eTCR of claim 88, wherein the scFv of claim 67 is linked to the C_{α} domain.

90. The eTCR of claim 88, wherein the scFv of claim 67 is linked to the C_{β} domain.

91. The eTCR of claim 88, wherein one scFv of claim 67 is linked to the C_{α} domain and one scFv of claim 67 is linked to the C_{β} domain.

92. A nucleic acid encoding an antibody or fragment thereof of claim 1.

93. The nucleic acid of claim 92, wherein the nucleic acid has the sequence as set forth in SEQ ID NOs: 14, 15, 16, 17, 18, 19, 20, 21, or 22, or has a sequence with at least 90% sequence identity to the sequence as set forth in SEQ ID NOs: 14, 15, 16, 17, 18, 19, 20, 21, or 22 wherein the encoded antibody or fragment thereof retains CD45 binding.

94. A cell genetically modified to express the antibody or fragment thereof of claim 1, the CAR of claim 84, or the eTCR of claim 88.

95. The cell of claim 94, wherein the cell is an immune cell.

96. The cell of claim 95, wherein the immune cell is a T cell, B cell, natural killer cell, or macrophage.

97. A composition comprising the antibody or fragment thereof of claim 1 or a nucleic acid of claim 92, and a pharmaceutically acceptable carrier.

98. A formulation comprising the cell of claim 94 and a pharmaceutically acceptable carrier.

99. A kit comprising the antibody or fragment thereof of claim 1, the multi-domain binding molecule of claim 40, the conjugate of claim 68, the CAR of claim 84, the eTCR of claim 88, or the nucleic acid of claim 92.

100. A method of treating a subject in need thereof comprising administering a therapeutically effective amount of the composition of claim 97 and/or the formulation of claim 98 thereby treating the subject in need thereof.

101. The method of claim 100, wherein the therapeutically effective amount provides a prophylactic or a therapeutic treatment against a hematologic disorder.

102. The method of claim 100, wherein the hematologic disorder comprises a hematopoietic malignancy.

103. The method of claim 102, wherein the hematologic malignancy comprises acute myelogenous leukemia (AML), acute lymphocytic leukemia (ALL), myelodysplastic syndromes with excess blasts, chronic lymphocytic leukemia (CLL), or chronic myelogenous leukemia (CML).

104. The method of claim 103, wherein the AML comprises relapsed or refractory AML.

105. The method of claim 100, wherein the therapeutically effective amount provides a conditioning treatment for a subject before the subject receives a hematopoietic cell transplant.

106. The method of claim 105, wherein the hematopoietic cell transplant comprises allogeneic hematopoietic cell transplant or autologous hematopoietic cell transplant.

107. The method of claim 106, wherein the transplanted hematopoietic cell are gene-edited.

108. The method of claim 100, wherein the subject is a human subject with an autoimmune disease, a metabolic disorder, or an inherited blood disorder.

109. The method of claim 100, wherein the administering is through intravenous, intradermal, intraarterial, intranodal, intravesicular, intrathecal, intraperitoneal, intraparenteral, intranasal, intralesional, intramuscular, oral, intrapulmonary, subcutaneous, or sublingual administering.
